(19) United States
(12) Patent Application Publication
(10) Pub. No.: US 2024/0390482 A1
(43) Pub. Date: Nov. 28, 2024

(54) HIV-1 ENVELOPE GLYCOPEPTIDE NANOPARTICLES AND THEIR USES

(71) Applicant: Duke University, Durham, NC (US)

(72) Inventors: Kevin Saunders, Durham, NC (US); Barton F. Haynes, Durham, NC (US); Christine Daniels, Durham, NC (US)

(21) Appl. No.: 18/280,935

(22) PCT Filed: Mar. 8, 2022
(86) PCT No.: PCT/US2022/019348

§ 371 Date: Sep. 7, 2023

Related U.S. Application Data
(60) Provisional application No. 63/158,074, filed on Mar. 8, 2021.

(51) Int. Cl. A61K 39/21; A61K 39/00; A61P 31/18; C07K 14/005

(52) U.S. Cl. CPC A61K 39/21; A61P 31/18; C07K 14/005; A61K 2039/55555; C07K 2319/00; C12N 2740/16022; C12N 2740/16034

(57) ABSTRACT

Provided are HIV-1 immunogens designed as glycopeptide nanoparticles and their use.

This application claims the benefit of and priority to U.S. Provisional Application No. 63/158,074 filed Mar. 8, 2021, which content is herein incorporated by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTEREST

This invention was made with government support under grant AI120801 and grant UM1-AI144371 from the NIH, NIAID, Division of AIDS. The government has certain rights in the invention.

### TECHNICAL FIELD

The invention comprises of a composition suitable for use in inducing anti-HIV-1 antibodies, such as to immunogenic compositions comprising envelope glycopeptides and nucleic acids to induce cross-reactive neutralizing antibodies and increase their breadth of coverage. The invention also encompasses methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and method for induction of glycan dependent immune response, for example without limitation cross-reactive (broadly) neutralizing (bn) Ab induction. In certain embodiments, the methods use compositions comprising HIV-1 immunogens designed as glycopeptide nanoparticle immunogens that aim to recapitulate the V3-glycan site. In non-limiting embodiments these glycopeptide immunogens present subsets of glycans found at positions 295, 301, 332, 339, 386, and 392. In certain embodiments, these are V3 glycan antibodies. Thus, in certain embodiments the invention provides HIV-1 peptide designs with multimerization and sequence optimization for enhanced targeting of HIV-1 V3 glycan sites.

In certain aspects the invention provides compositions comprising a selection of HIV-1 envelopes/peptides thereof and/or nucleic acids encoding these for example but not limited to designs as described herein.

In non-limiting embodiments, the invention provides a recombinant HIV-1 glycopeptide of Table 2, Table 3, Table 4, FIG. 6 or FIG. 7 or further described at least in section “Multimeric glycopeptide immunogen” or the accompanying examples. A nucleic acid encoding any of the recombinant glycopeptide. In certain embodiments, the nucleic acids an mRNA formulated for use as a pharmaceutical composition. A skilled artisan appreciates that the sequences in FIG. 6 include various linkers and self-assembling proteins so as to form multimeric complexes/nanoparticles. The sequences of the glycopeptide can be fused, genetically or otherwise, to any other self-assembling protein which forms multimeric complexes/nanoparticles.

In certain embodiments, the inventive designs comprise modifications, including without limitation linkers between the glycopeptide and ferritin designed to optimize ferritin nanoparticle assembly.

A composition comprising any one of the inventive peptide designs or nucleic acid sequences encoding the same. In certain embodiments, the nucleic acid is mRNA. In certain embodiments, the mRNA is comprised in a lipid nano-particle (LNP).

Compositions comprising a nanoparticle which comprises any one of the glycopeptides of the invention.

The composition of any of the claims, wherein the nanoparticle is ferritin self-assembling nanoparticle.

A method of inducing an immune response in a subject comprising administering an immunogenic composition comprising any one of the peptide designs of the invention. In certain embodiments, the composition is administered as a prime and/or a boost. In certain embodiments, the composition comprises nanoparticles. In certain embodiments, methods of the invention further comprise administering an adjuvant.

A composition comprising a plurality of nanoparticles comprising a plurality of the envelopes/glycopeptides of the invention. In non-limiting embodiments, the glycopeptides of the invention are multimeric when comprised in a nano-particle. The nanoparticle size is suitable for delivery. In non-liming embodiments the nanoparticles are ferritin based nano-particles.

In certain aspects the invention provides a recombinant HIV-1 glycopeptide selected from the glycopeptides listed in Table 2, Table 3, Table 4, FIG. 6 or FIG. 7.

In certain embodiments, the glycopeptide is genetically fused via a linker to a self-assembling protein. In certain embodiments, the self-assembling protein is ferritin.

In certain aspects, the invention provides a composition comprising a nanoparticle and a carrier, wherein the nanoparticle comprises any one of the glycopeptides of the invention. In certain embodiments, the nanoparticle is ferritin self-assembling nanoparticle.

In certain embodiments of the nanoparticles, the nanoparticle comprises 1-24 glycopeptide copies.

In certain embodiments the compositions and methods of the invention further comprise an adjuvant. In certain embodiments, the adjuvant is LNP, TLR 7/8 antagonist, alum or a combination thereof.

In certain aspects, the invention provides a method of inducing an HIV-1 immune response in a subject comprising administering an immunogenic composition comprising any one of the recombinant glycopeptides of the invention or compositions of the invention.

In certain embodiments, the composition is administered as a prime. In certain embodiments, the composition is administered as a boost.

In certain aspects the invention provides a nucleic acid encoding any of the recombinant glycopeptides of the invention. Provides are also compositions comprising the nucleic acids of the invention and a carrier. In certain embodiments, the nucleic acid is an mRNA. In certain embodiments, the nucleic acid compositions comprise an adjuvant. In certain embodiment the nucleic acids are encapsulated in lipid nanoparticles (LNPs). In certain aspects, the invention provides a method of inducing an immune response in a subject comprising administering an immunogenic composition comprising the nucleic acid of the invention or compositions comprising these.

In certain aspects, the invention provides a recombinant fusion protein sequence comprising in order a V3glycopeptide, a peptide linker and a self-assembling protein, wherein the fusion protein is comprised in a multimeric protein complex. In non-limiting embodiments the V3 glycopeptides is listed in Table 2. The recombinant fusion protein sequences where the protein including the V3 glycopeptide is recombinantly produced and not synthetic. In certain embodiments, the glycopeptides are listed in Table 2. In certain embodiments the recombinant fusion protein is listed in Table 3, Table 4, FIG. 6 or FIG. 7. In non-limiting embodiments, the amino acid sequence is listed in FIG. 7C.

Any suitable linker can be used. In certain embodiments, the linker is described in Table 1, is included in designs listed in Table 3, or is included in designs listed in Table 4. In certain embodiments, the fusion design does not have a linker. In certain embodiments, the linker is LPXTGGGGGGSG or GSGLPXTGGGGG, wherein in some embodiments X is E. In certain embodiments, the linker is GGS (n), wherein n=1, 2, or 3.

In certain embodiments, the linker is GGS (n) EKAAKAEEAAR (PP) the linker is EKAAKAEEAAR(PP)GGS(n) wherein n=1, 2, or 3.

In certain embodiments, the protein further comprises a T helper epitope. In certain embodiments, the protein further comprises at least one T helper epitope. In certain embodiments, the protein further comprises one, two, three, four or five T helper epitopes. In non-limiting embodiments the T helper epitope is listed in Table 5. The T-helper epitope can be placed in any suitable location in the fusion protein. In certain embodiments, the T helper epitope(s) are comprised in the immunogenic compositions comprising the inventive glycopeptides.

In certain embodiments, the self-assembling protein is ferritin and the multimeric protein complex is ferritin nanoparticle.

In certain aspects, the invention provides a nucleic acid encoding the recombinant fusion protein of the invention. In certain embodiments, the nucleic acid is an mRNA. In certain embodiments, the mRNA is modified mRNA and encoded in LNPs. In certain embodiments, the modified mRNA comprises pseudouridine.

In certain aspects, the invention provides a multimeric protein complex comprising a recombinant fusion protein sequence comprising in order a V3 glycopeptide, a peptide linker and a self-assembling protein that forms the multimeric protein complex. In certain embodiments, the linker is described in Table 4. In certain embodiments, the self-assembling protein is ferritin and the multimeric protein complex is ferritin nanoparticle. In certain embodiments, the linker is LPXTGGGGGGSG or GSGLPXTGGGGG, wherein in some embodiments X is E. In certain embodiments, the linker is [GGS] (n), wherein n=1, 2, or 3. In certain embodiments, the linker is [GGS] (n) EKAAKAEEAAR (PP) the linker is EKAAKAEEAAR(PP)[GGS](n) wherein n=1, 2, or 3. In certain embodiments the recombinant fusion protein is listed in Table 3, Table 4, FIG. 6 or FIG. 7. In non-limiting embodiments, the amino acid sequence is listed in FIG. 7C.

In certain aspects, the invention provides a composition comprising a recombinantly produced fusion protein of the invention and a pharmaceutically acceptable carrier. In certain embodiments the compositions comprise an adjuvant. In certain embodiments, the adjuvant is LNP, TLR 7/8 antagonist, alum or a combination thereof. In certain embodiments the recombinant fusion protein is listed in Table 3, Table 4, FIG. 6 or FIG. 7. In non-limiting embodiments, the amino acid sequence is listed in FIG. 7C.

In certain aspects, the invention provides a composition comprising a recombinantly produced fusion protein and a pharmaceutically acceptable carrier, wherein the self-assembling protein is ferritin and the multimeric protein complex is ferritin nanoparticle.

In certain aspects, the invention provides a composition comprising a nucleic acid sequence encoding the recombinantly produced fusion protein and a pharmaceutically acceptable carrier. Non-limiting embodiments of nucleic acids encoding the recombinant fusion proteins of the invention are shown in FIG. 6 and FIG. 7.

In certain aspects, the invention provides a virus-like particle comprising any one of the recombinant fusion protein of the invention.

In certain aspects, the invention provides a host cell comprising a nucleic acid molecule encoding a recombinant fusion protein of the invention.

In certain aspects, the invention provides an immunogenic composition comprising any of the recombinant fusion proteins, nucleic acids encoding these recombinant fusion proteins, multimeric protein complex or VLP comprising recombinant fusion proteins of the invention and a pharmaceutically acceptable carrier and an adjuvant.

In certain aspects, the invention provides methods for inducing an immune response to HIV-1 in a subject, comprising administering to the subject an effective amount of any of the recombinant fusion proteins of the invention, nucleic acids or combinations of nucleic acids encoding these recombinant fusion proteins, multimeric protein complexes, or the immunogenic compositions of the invention in an amount sufficient to induce an immune response. In certain embodiments the induced immune response comprises V3 glycan directed antibodies. In certain embodiments, the induced immune response comprises FDG antibodies.

In certain embodiments, the effective amount of any of the recombinant fusion proteins of the invention, nucleic acids or combinations of nucleic acids encoding these recombinant fusion proteins, multimeric protein complexes, or the immunogenic composition of the preceding claims is administered as a boost. In certain embodiments, the method comprises multiple boost with the same immunogen.

### BRIEF DESCRIPTION OF THE DRAWINGS

FIG. 1 shows H. pylori nanoparticles used to array multiple copies of glycopeptide nanoparticles. Each nanoparticle is composed of 24 subunits to which glycopeptides can be added appended. (Left) Structure of H. pylori ferritin. (Middle and right) Negative stain EM analysis of Man9V3 glycopeptide ferritin nanoparticles.

FIG. 2 shows Design of recombinant Man9V3 glycopeptide. Arrows indicate design modifications. Glycans are shown in blue stick structure. Peptide is shown as red ribbon structure. Cysteines that hold the loop together are indicated by Cys in magenta.

FIG. 3A-B shows Design of recombinant TetraglycoV3 glycopeptide. Magenta arrow indicates the additional 339 glycan that was added to the glycopeptide design compared to the ManV3 glycopeptide. FIG. 3B shows Amino acid alignment of various glycoV4 designs. Dashes indicate mismatch sequences.

FIG. 4 shows structure of the recombinant glycoV4 glycopeptide immunogen with key N386 and N392 glycans indicated. Glycans are shown in blue sticks. the truncated flexible tip is shown as a dotted line.

FIG. 5 shows sequential vaccine to affinity mature FDG bnAbs. Glycopeptide mimics of the glycan-V3 site can guide antibody affinity maturation to recognize more glycosylation sites and more glycan types on Env, resulting in FDG antibodies that broadly neutralize HIV-1. Immunogens that guide each step are written above each arrow.

FIG. 6A-6B show non-limiting embodiments of nucleic acid (6A-1 through 6A-13) and amino acid sequences of peptide designs of the invention. The signal peptide is underlined in the amino acid sequences of FIGS. 6B-1 through 6B-13.

FIG. 7A-7C show non-limiting embodiments of nucleic acid and amino acid sequences of peptide designs of the invention where the peptide designs comprise T-cell helper epitopes. Without being bound by theory, addition of the T cell helper epitopes increases the magnitude and/or breadth of the immune response. In some embodiments the designs include any other suitable T cell helper epitope or combination thereof. FIG. 7A-1 through 7A-24 show nucleic acid sequence. FIG. 7B-1 through 7B-24 show amino acid sequences which comprise the signal peptide and purification tags. FIG. 7C-1 through 7C-24 show amino acid sequences which do not include the signal peptide and purification tags.

FIG. 8 shows Design of V3-Glycan Epitope-Focused Glycopeptide Immunogens. Design and visualization of Glycopeptide Nanoparticle Immunogens 9slides 4-8): Ribbon structures showing peptide (V3 red, V4 green) and the positions of attached glycans (blue) of glycopeptide constructs. Arrows indicate regions that were modified. Immunodominant regions were excluded from design. Assembled nanoparticles were visualized by negative stain electron microscopy. Reference-free 2D class averages were generated from raw micrographs. Enlarged image shown in bottom right. Scale bar 200 nm.

FIG. 9 shows Visualization of V3 Loop Glycopeptide Nanoparticles.

FIG. 10 shows V4 Loop Glycopeptide Nanoparticle.

FIG. 11 shows Multimerization of V3 Epitope using Ferritin Nanoparticles. Engineering of Glycopeptide Nanoparticles: Schematic representation of glycopeptide epitope being applied to ferritin nanoparticle scaffold to generate a fusion gene. A 6× His tag was appended to the C terminus of the fusion gene for purifying the gene of interest.

FIG. 12 shows Production of Glycopeptide Nanoparticles.

FIG. 13A-B show Expression of V3 Glycopeptide Nanoparticles. Figure shows Analytical Size Exclusion Chromatography. Expression and Purification of Glycopeptide nanoparticles: Glycopeptide nanoparticles were purified using antibody affinity chromatography and size exclusion chromatography. Analytical size exclusion of collected protein showed the purified protein was a single peak of homogenous protein consistent with the size of a nanoparticle.

FIG. 14A-C show Expression of V3 Glycopeptide Nanoparticles. Figure shows Analytical Size Exclusion Chromatography.

FIG. 15A-B shows that V3 Glycopeptides Are Efficiently Incorporated into Ferritin Nanoparticles. Figure shows Negative Stain Electron Microscopy. Visualization of assembled glycopeptide nanoparticles: To ensure incorporation of glycopeptide sequences did not disrupt nanoparticle formation we used negative stain electron microscopy to examine morphology. Purified nanoparticle samples were applied to a mesh carbon electron microscopy grip and stained with 0.5% uranyl fomate. Enlarged images show 2D class averages generated from raw micrographs. Each construct design yielded homogenous assembled particles.

FIG. 16A-C shows V4 Glycopeptides Are Efficiently Incorporated into Ferritin Nanoparticles. Figure shows Negative Stain Electron Microscopy. Visualization of assembled glycopeptide nanoparticles: To ensure incorporation of glycopeptide sequences did not disrupt nanoparticle formation we used negative stain electron microscopy to examine morphology. Purified nanoparticle samples were applied to a mesh carbon electron microscopy grip and stained with 0.5% uranyl fomate. Enlarged images show 2D class averages generated from raw micrographs. Each construct design yielded homogenous assembled particles.

FIG. 17A-B show Site-Specific Percent Composition of Glycans on Glycopeptide Nanoparticles. Glycoprofiling of glycopeptide nanoparticles: Relative high mannose (red) and processed glycan (blue) composition represented as bars. Slide 15 shows relative amounts of various high mannose glycan present at each position. Both constructs show formation of a glycan cluster surrounding position N332.

FIG. 18A-C show Relative Abundance of High Mannose species Expressed on Glycopeptides.

FIG. 19A-B show Man₉V3-ferritin_3C-His Recapitulates Env SOSIP Trimer Binding to V3 Glycan Antibodies. Figure shows Biolayer Interferometry. Figure shows evaluation of Glycopeptide nanoparticle antigenicity: Glycopeptides showed strong reactivity with neutralizing antibodies that target the V3 glycan epitope and failed to react with antibodies with specificities for poorly conserved peptide regions. FIG. 19A shows Man₉V3 has high association with V3 Glycan bnAbs but not linear V3 mAbs. FIG. 19B shows SOSIP Trimer is bound by both V3-glycan and the linear V3 antibody tested.

FIG. 20A-B show Man₉V3-ferritin_3C-His Recapitulates Env SOSIP Trimer Binding to V3 Glycan Antibodies. Figure shows Biolayer Interferometry.

FIG. 21A-C show TetraglycoV3-ferritin_3C-His Nanoparticles are Bound with HIV Broadly Neutralizing Antibodies. Figure shows Biolayer Interferometry. Red vertical lines indicate response at end of association.

FIG. 22 shows GlycoV4-FL-ferritin_3C-His Nanoparticles are Bound with HIV Broadly Neutralizing Antibodies. Data show Biolayer Interferometry Binding Summary.

FIG. 23A-C show GlycoV4-FL-ferritin_3C-His Nanoparticles are Bound with HIV Broadly Neutralizing Antibodies. Figure shows Biolayer Interferometry. Red vertical lines indicate response at end of association.

FIG. 24 shows GlycoV4-FL-ferritin_3C-His Nanoparticles are Bound with HIV Broadly Neutralizing Antibodies. Figure shows Biolayer Interferometry Binding Summary.

FIG. 25A-F shows Antigenicity of Glycopeptide Nanoparticles Expressing Different Glycoforms. Figure shows Biolayer Interferometry Binding.

FIG. 26A-H shows Antigenicity of Glycopeptide Nanoparticles Expressing Different Glycoforms. Figure shows Biolayer Interferometry Binding.

FIG. 27A-B show Fab Dimerized Glycan Antibody Binding to Manv3NP vs Manv3NP/Kif.

FIG. 28A-B show Fab Dimerized Glycan Antibody Binding to Manv3NP vs Manv3NP/Kif. Antibodies are listed on the x-axis.

FIG. 29 shows Man9V3-ferritin: untreated and Kif-treated.

FIG. 30A-B show that V3 Glycopeptide Nanoparticles are Recognized by V13 Glycan Targeting Antibodies. Figure shows that Kif treatment enhances binding.

FIG. 31A-B show that V3 Glycopeptide Nanoparticles are not Recognized by V1/V2 Glycan Targeting Antibodies.

FIG. 32 shows that Glycopeptide Nanoparticles are Recognized by Extended Loop V3-Glycan mAbs and that Man₉V3 Nanoparticles bind V3 Glycan nAbs.

FIG. 33 shows that Glycopeptide Nanoparticles are Recognized by Domain Swapped mAb 2G12—Man₉V3 Nanoparticles are recognized by 2G12.

FIG. 34 shows that Glycopeptide Nanoparticles are Recognized by Fab Dimerized Glycan mAbs—Man₉V3 Nanoparticles are recognized by FDG nAbs.

FIG. 35 shows MangGlcNAc2-enrichment with Kifunensine Improves Man₉ V3-ferritin_3C-His Antigenicity—Kif treatment improves glycan-dependent antibody binding to GlycoV3 NP.

FIG. 36 shows V3 and V4 Glycopeptide Nanoparticles Are Antigenic for Neutralizing and not Non-Neutralizing Antibodies.

FIG. 37 shows PGT128 Lineage Antibodies Affinity Mature to Bind V3-Glycans-ELISA binding of PGT128 lineage antibodies to Man₉V3-ferritin_3C-His.

FIG. 38 shows PGT121 Lineage Antibodies Affinity Mature to Bind V3-Glycans--ELISA binding of PGT121 lineage antibodies to Man9V3-ferritin_3C-His.

FIG. 39 shows DH270 Lineage Antibodies Affinity Mature to Bind V3-Glycans

FIG. 40A-B show DH270 Lineage Antibodies Affinity Mature to Bind V3-Glycans.

FIG. 41 shows that Man9V3-ferritin is Thermodynamically Stable. Figure shows Differential Scanning Fluorescence. Thermostability of Man9V3-ferritin glycopeptide nanoparticles: Man9V3-ferritin demonstrated the highest melting temperature when compared to monomeric CON-S Env and trimeric CON-S. Man9V3 was able to withstand higher temperatures before unfolding.

FIG. 42 shows Immunization Schedule for one embodiment of an animal study to study immunogenicity of glycopeptide nanoparticles: Schematic of immunization schedule shown. Group 2 rabbits elicited high titers of autologous antibodies against priming immunogen after 3 dose. Boosting with glycopeptide nanoparticles maintained titers for 12 weeks after final SOSIP boost. 10/10 rabbits generated responses that can discriminate between glycosylated peptide constructs and identical constructs lacking glycans. Protein dose: 100 μg Man₉V3-ferritin. Route: Intramuscular. Adjuvant: 25 μg GLA-SE.

FIG. 43 shows Man₉V3-NP Boost Elicits High Titers of Autologous Abs in Rabbits—ELISA of serum from Man₉V3-ferritin_3C-His boosted animals to JRFL SOSIP Trimer.

FIG. 44 shows Man₉V3-NP Boost Elicits Man₉V3 Peptide Reactive Antibodies in 10 out of 10 Rabbits.

FIG. 45 shows Man₉V3-NP Boost Elicits Autologous Nanoparticle Antibodies in 10 out of 10 Rabbits.

FIG. 46A-B show Man₉V3-ferritin 3C-His Boost Maintains Serum Antibody Binding Titers for 12 weeks Post Immunization.

FIG. 47A-D shows CH848 Env Prime Elicits Antibodies That React with Glycopeptide Nanoparticles. Env Prime elicits antibodies that recognize glycopeptides: Binding data from mouse sera from two separate studies using different strategies. Env prime elicited antibodies that can bind to heterogeneous glycoforms and complex glycans at multiple glcosylation sites.

FIG. 48A-C show CH848 Env Prime Elicits Antibodies That React with Glycopeptide Nanoparticles

FIG. 49 shows fuman antibodies isolated from infected individuals that react with Glycopeptide Nanoparticles. Glycopeptides may be used to boost preexisting antibody responses elicited by Env prime. The kif treated nanoparticles display the strongest and broadest reactivity in this regard.

FIG. 50A-C show Schematic of the generation of glycopeptide nanoparticles. FIG. 50A and 50B show Cartoon structure of glycopeptides with glycans shown in blue and peptide shown in red. Glycan position in envelope is shown in blue. Glycopeptides were genetically fused to ferritin subunits resulting in glycopeptide nanoparticle proteins that can be visualized by negative stain electron microscopy.

FIG. 51A-D show Negative stain electron microscopy of different glycopeptide nanoparticles. Man9V3 glycopeptide nanoparticle is enriched for Man9 glycosylation by production in the presence of kifunensine. GlycoV3 is the same protein sequence as Man9V3 but without glycosylation restriction. Tetraglycone and glycoV4min2 are heterogeneously glycosylated peptide nanoparticles representing the V3 and V4 regions, respectively.

FIG. 52A-B show Man9V3 glycopeptide nanoparticles display high mannose glycosylated peptides. Mass spectrometry analysis of glycans present on Man9V3 nanoparticles show a predominance of Man9GlcNAc2 glycosylation at N295, N301, and N332 sites on the glycopeptide.

FIG. 53 shows Glycopeptide Nanoparticle Antigenicity. V3-glycan bnAb and Fab-dimerized glycan (FDG) antibody ELISA binding titers to mannose-enriched glycopeptide nanoparticles. ELISA binding titers are shown as log area-the-curve (Log AUC). Man9V3 and Tetraglycone glycopeptide nanoparticles are enriched for Man₉GlcNAc₂ glycosylation by production in the presence of kifunensine. Tetraglycone nanoparticles show higher binding titers for DH270, BG18, DH851.3 broadly neutralizing antibodies compared to Man9V3 nanoparticle.

FIG. 54 shows ELISA binding titers to different glycopeptide nanoparticles for various types of glycan antibodies. ELISA binding titers are shown as log area-the-curve (Log AUC). Fab dimerized glycan (FDG) antibodies are classified as Type I, II, or III based on their neutralization breadth. Man9V3 glycopeptide nanoparticle is enriched for Man9 glycosylation by production in the presence of kifunensine. GlycoV3 is the same protein sequence as Man9V3 but without glycosylation restriction. Tetraglycone and glycoV4 (FL, min1, and min2) are heterogeneously glycosylated peptide nanoparticles representing the V3 and V4 regions respectively.

FIG. 55 shows PGT128 Lineage antibodies affinity mature to bind Man9V3 nanoparticles. ELISA binding titers are shown as log area-the-curve (Log AUC). The darker the red the stronger the binding. Antibodies are ordered left to right as unmutated to most mutated.

FIG. 56 shows PGT121 Lineage antibodies affinity mature to bind Man9V3 nanoparticles. ELISA binding titers are shown as log area-the-curve (Log AUC). The darker the red the stronger the binding. Antibodies are ordered left to right as unmutated to highly mutated.

FIG. 57 shows DH270 lineage antibodies bind to Man9V3 nanoparticles after affinity maturation. ELISA binding titers are shown as log area-the-curve (Log AUC). Antibodies are ordered left to right in the order of increasing divergence from the unmutated common ancestor (UCA) that is inferred to have given rise to the lineage.

FIG. 58A-B shows Rabbit vaccination schema. FIG. 58A. Immunization schedule: Route: Intramuscularly; Dose: 50 mcg; Adjuvant: 3M-052-Alum; Model: New Zealand white rabbits (n=5). B. Immunization groups showing sequential immunogens.

FIG. 59 shows Serum neutralization of JR-FL pseudovirus infection of TZM-bl cells. Serum was collected two weeks after the fourth immunization of each rabbit. Neutralization of JR-FL, JR-FL lacking a glycan at position N332 (N332A), or JR-FL lacking 4 glycans in the first variable region (JR-FL_AV1.PNGS) was assessed for each rabbit. Each symbol represents an individual rabbit (n=5 per group). Bars indicate group geometric mean with error shown as geometric standard deviation. The dashed line shows the limit of detection at a titer of 20.

FIG. 60 shows Vaccine induction of serum DH270.1 blocking antibodies in rabbits. Shown is the blocking of V3-glycan neutralizing antibody 2G12 binding to JR-FL_ΔV1.PNGS SOSIPV6 Env trimer by rabbit serum from before vaccination (Prebleed, blue) or after the fourth immunization (Postbleed 4, red). Values for each rabbit at the two timepoints are connected with a black line. Each symbol represents an individual rabbit (n=5 per group). Representative results are shown from three independent experiments.

FIG. 61 shows Vaccine induction of serum 2G12 blocking antibodies in rabbits. Shown is the blocking of glycan neutralizing antibody 2G12 binding to JR-FL_ΔV1.PNGS SOSIPv6 Env trimer by rabbit serum from before vaccination (Prebleed, blue) or after the fourth immunization (Postbleed 4, red). Values for each rabbit at the two timepoints are connected with a black line. Each symbol represents an individual rabbit (n=5 per group). Representative results are shown from three independent experiments.

FIG. 62 shows Serum 2G12 blocking antibodies elicited by vaccination are moderately affected by glycosylation and sequence changes in the first variable region. Shown is the blocking of glycan neutralizing antibody 2G12 binding to JR-FL Env trimers with various V1 sequences by rabbit serum from before vaccination (Prebleed, blue) or after the fourth immunization (Postbleed 4, red). Serum antibody blocking of 2G12 binding to wildtype JR-FL (SOSIPV6), JR-FL lacking 4 glycans in its natural first variable region (JR-FL_ΔV1.PNGS), or JR-FL with its first variable region exchanged for a V1 that is from a heterologous virus (D1305V1). The heterologous V1 is 8 amino acids shorter (17 amino acids) than the wildtype JR-FL V1 and lacks V1 glycans N-terminal to position 156. Values for each rabbit at the two timepoints are connected with a black line. Each symbol represents an individual rabbit (n=5 per group). Representative results are shown from three independent experiments.

FIG. 63A-C show groups of rabbits with blocking antibodies against 2G12 and DH270.1 and serum neutralization exhibit IgG binding to JR-FL_ΔV1.PNGS SOSIPV6 Env trimer. IgG binding to JR-FL_ΔV1.PNGS SOSIPV6 Env trimer arose after 1 or 2 JR-FL trimer immunizations but did not arise after 2 Man9V3 NP immunizations. Arrows indicate immunization timepoints. Blue lines indicate IgG binding titers and red lines indicate IgM binding titers. Each symbol represents the mean for the group of rabbits (n=5 per group) and standard error.

FIG. 64A-L show Glycopeptide nanoparticle immunization elicits IgG responses against the Man9V3 and aglycone peptides, but JR-FL Env alone does not. Immunogens are shown on the x-axis. IgG binding to aglycone and Man9V3 peptides was equivalent showing IgG reactivity was primarily to the peptide. After JR-FL_ΔV1.PNGS SOSIPV6 immunization, IgG and IgM responses were not elicited against either peptide. However, IgG responses were detectable if the animals were boosted with glycoV3 nanoparticle. Arrows indicate immunization timepoints. Blue lines indicate IgG binding titers and red lines indicate IgM binding titers. Each symbol represents the mean for the group of rabbits (n=5 per group) and standard error.

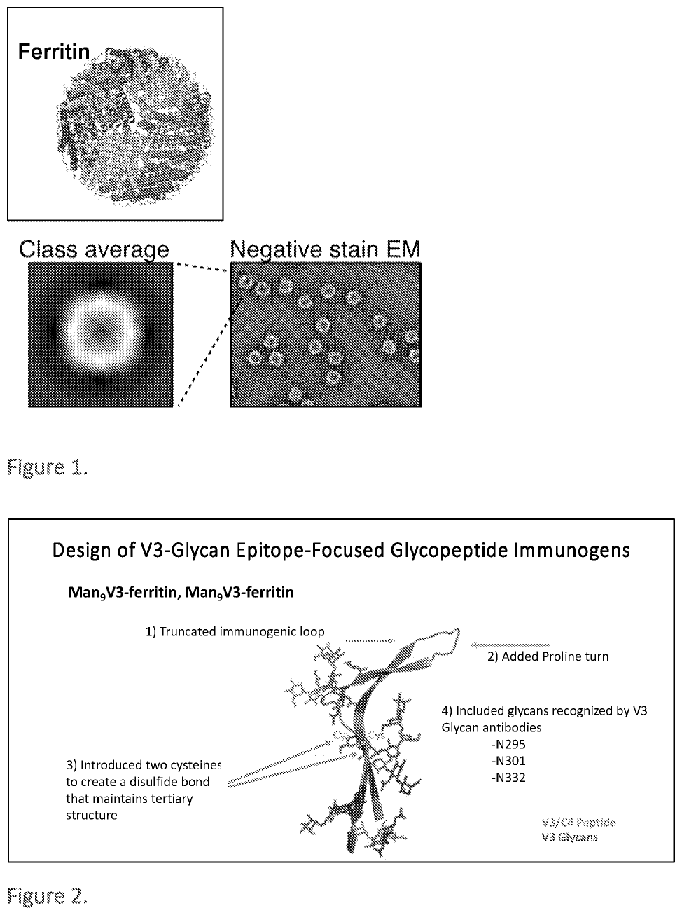

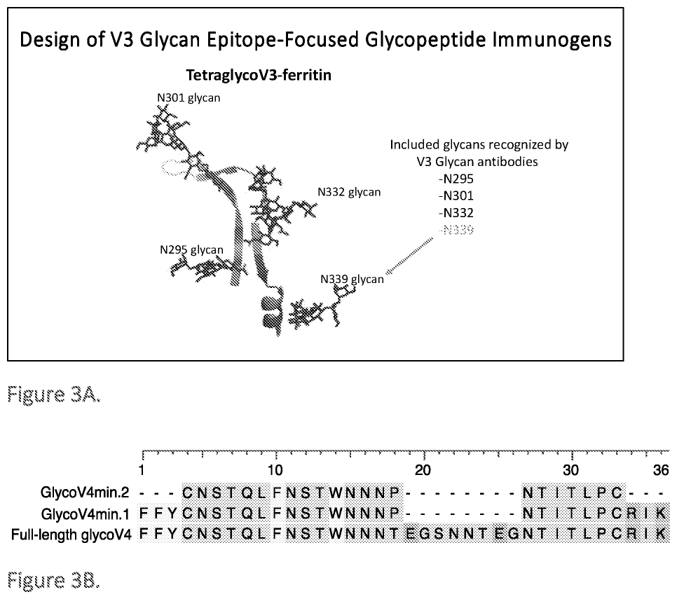

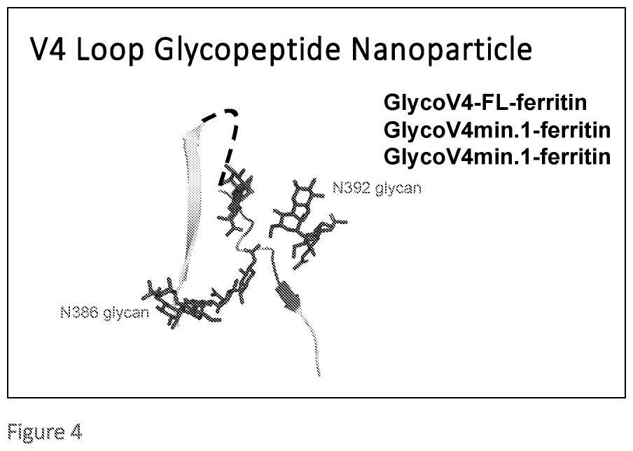

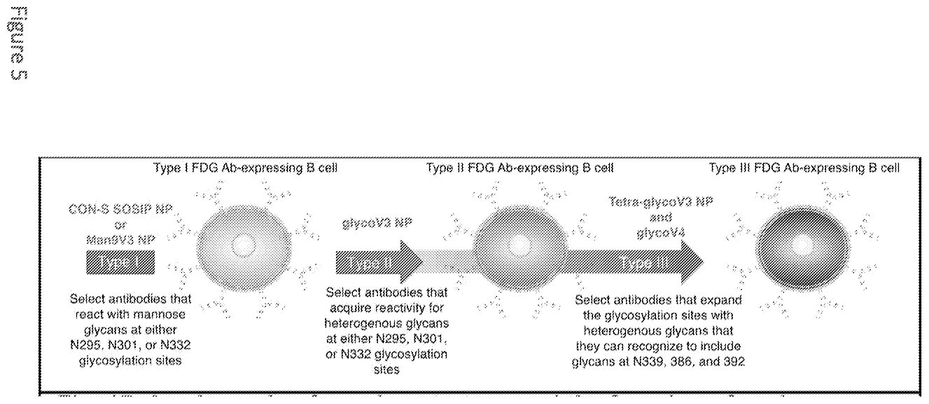

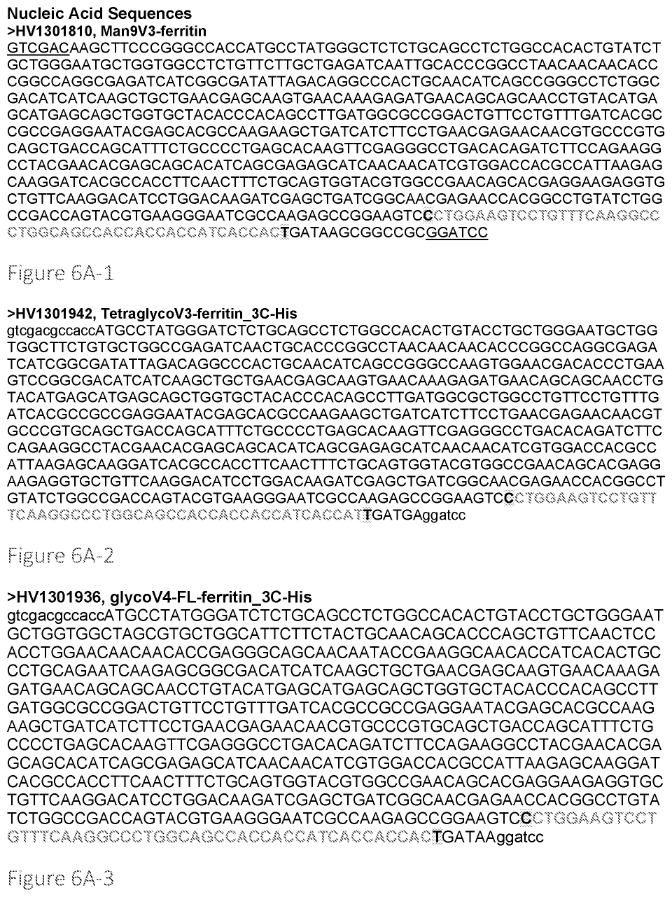

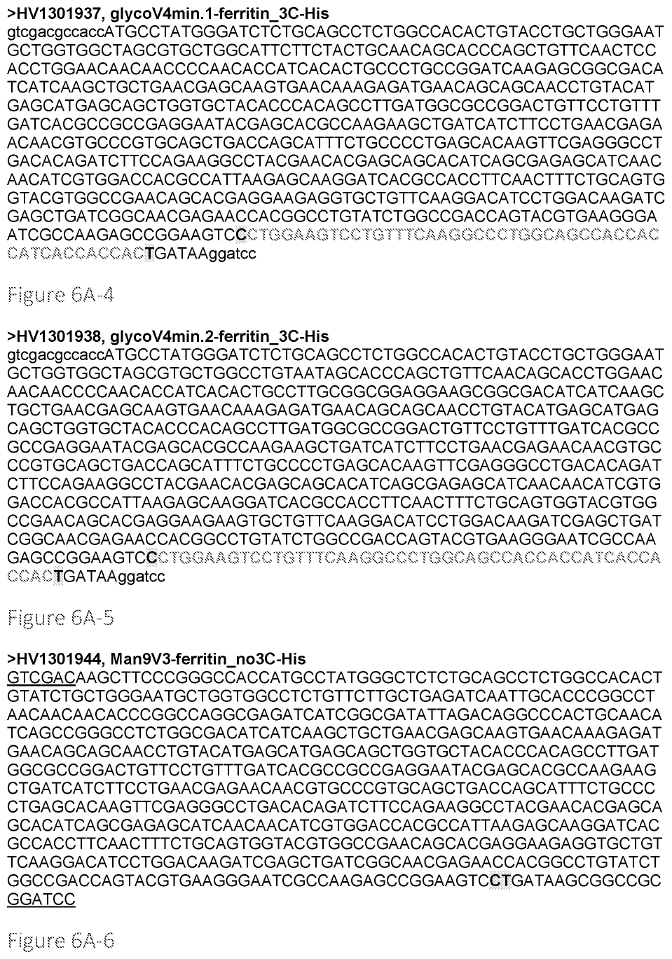

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254:225-244, 2013). BnAbs are protective in rhesus macaques against SHIV challenge, but as yet, are not induced by current vaccines.

In certain aspect, the invention provides compositions for immunizations to induce different groups of HIV-1 neutralizing antibodies—see section antibody targets of the immunogen.

Antibody responses to glycoproteins. Glycoproteins decorate the outer surface of viruses, bacteria, and fungi making them targets for the immune system. Glycans, or carbohydrate molecules with glycosidic linkages, are naturally attached to human proteins as well. Thus, the immune system must limit glycan-reactive antibody responses to those that react with specific pathogen glycoproteins, but not self-proteins. For this reason, glycan-reactive antibodies are only a small fraction of the human antibody repertoire. The limited number of glycan-reactive antibodies results in poor immunogenicity of glycan-dependent epitopes on pathogen proteins. The poor immunogenicity of glycan-dependent epitopes on viral glycoproteins necessitates vaccine strategies to augment this antibody response.

To augment glycan-dependent antibody responses, we have taken an immuno-focusing approach. This approach that aims to eliminate dominant antibody responses to undesired epitopes, while heightening antibody responses to glycan-dependent epitopes of interest. The invention identifies glycan-dependent epitopes in larger proteins, and isolates those glycan-dependent epitopes as smaller antigens encompassing just the epitope of interest. Essential to the elicitation of relevant glycan-reactive antibodies is for the glycans to be presented with the appropriate spacing and arrangement as found in the larger natural glycoprotein. Thus, protein structural and biochemical considerations must be part of antigen design. The smaller antigen derived from the glycan-dependent epitope of interest can be used as an immunogen to elicit a focused immune response to the glycan-dependent epitope without eliciting B and T cell responses to portions of the pathogen glycoprotein distinct from the glycan-dependent epitope of interest. Once the antibodies that can recognize the epitope of interest are magnified, their reactivities can be honed to specific types of glycans or numbers of glycans.

Viral antigen selection. Viral pathogens express glycoproteins. Human immunodeficiency virus subtype 1 (HIV-1) is a notable example of one of these pathogens. The envelope glycoprotein on the surface of HIV-1 is the target for neutralizing antibodies. The molecular mass of HIV-1 envelope is 50% glycan. The glycans of HIV-1 envelope leave very little protein surface uncovered. Antibodies aiming to bind HIV-1 envelope must directly contact glycan or navigate around the glycans to optimally engage most neutralizing epitopes. Conserved neutralizing glycan-dependent epitopes exist on HIV-1 envelope. These include the V1V2-glycan site, V3-glycan site, gp120-gp41 interface, CD4 binding site, and the silent face. The V3-glycan site is composed of a collection of glycans attached to asparagines at positions 295, 301, 332, 339, 386, and 392. Antibodies that bind the V3-glycan site contact one or more of these glycans as well as the proximal peptide. V3-glycan antibodies can protect against infection in animal models, and reduce viral load in humans. Thus, focusing the response to the V3-glycan site a principal goal of the immunogen design of the invention.

Herein, we describe glycopeptide nanoparticle immunogens that aim to recapitulate the V3-glycan site. These glycopeptide immunogens present subsets of glycans found at positions 295, 301, 332, 339, 386, and 392. By presenting these glycans and adjacent protein to the immune system one can elicit a focused immune response against conserved, neutralizing peptide-glycan epitopes found on the HIV-1 envelope. Antibodies that bind neutralizing epitopes can neutralize HIV-1 and, without wishing to be bound by theory, provide protection from infection. Thus, the V3-glycan site from HIV-1 envelope was selected to elicit protective HIV-1 immunity. In addition to the glycans listed herein, this site encompasses peptide sequence at the base of the third variable region, third constant region, and fourth variable region. Since many viruses, such as SARS-COV, SARS-COV-2, Lassa Fever, and Hepatitis C have glycosylated viral proteins this same approach of identifying a glycan-dependent neutralizing epitope and expressing it alone can be applied to viral glycoproteins from these other viruses.

Antibody targets of the immunogen. The V3-glycan directed immunogens described here were designed to target three different groups of HIV-1 neutralizing antibodies. The group of antibodies includes the V3-glycan broadly neutralizing antibodies. These antibodies bind the glycan attached to asparagine 332 and surrounding peptide and glycan. They are potent neutralizing antibodies with 50-70% neutralization breadth. Specific examples of these antibodies include PGT128, PGT124, and DH270.6. The second group of antibodies targeted by this immunogen design includes only one antibody called 2G12. 2G12 is broadly neutralizing and binds the glycans 295, 332, and potentially 339 or 386 depending on specific virus sequences. 2G12 is notable due to its domain-swapped antibody structure, and its binding solely to glycans without any peptide contact. The group of targeted antibodies include the Fab-dimerized glycan antibodies. These antibodies primarily recognize glycan on the HIV-1 envelope. They are distinguished by their antibody morphology that can adopt both an I-shape or a Y-shape where the Fab arms of the antibody touch or are apart. Fab-dimerized glycan (FDG) antibodies can develop moderate neutralization breadth and represent a third group of potentially protective antibodies.

Enhancing the immunogenicity of minimal immunogens. Reducing the protein size down to only the glycan-dependent epitope of interest generates small glycopeptide antigens. Small peptides tend to not cross-link B cell receptors, which results in poor B cell activation and expansion. Poor B cell activation leads to low immunogenicity of such antigen designs. To augment the immunogenicity of the glycopeptide minimal immunogen we have arrayed them on the surface of protein nanoparticles. For example, 24-subunit ferritin nanoparticles can have the glycopeptide that recapitulates the glycan-dependent envelope epitope attached to its N-terminus separated by a short glycine-serine linker. The ferritin nanoparticle can be derived from different species. For example, Trichoplusia ni ferritin sequence can be used to encode the same or different glycopeptides on the heavy and light chains of the nanoparticle. Other examples of nanoparticles that can be used include encapsulins from various bacterial species.

In one aspect the invention provides a composition for a prime boost immunization regimen comprising any one of the glycopeptide designs described herein, administered as a glycopeptide nanoparticle or any combination thereof wherein the glycopeptide nanoparticle is a prime or boost immunogen. In certain embodiments the composition for a prime boost immunization regimen comprises one or more glycopeptides described herein.

In certain embodiments, the compositions include nucleic acid, as DNA and/or RNA, or proteins immunogens alone or in any combination. In certain embodiments, the methods encompass genetic, as DNA and/or RNA, immunization alone or in combination with envelope glycopeptide(s).

In some embodiments the antigens are nucleic acids, including but not limited to mRNAs which can be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In embodiments the nucleic acid encoding a glycopeptide is operably linked to a promoter inserted an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the glycopeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In certain embodiments, glycopeptides of the invention are multimerized, for example attached to a particle such that multiple copies of the glycopeptide are attached and the multimerized glycopeptide is prepared and formulated for immunization in a human. In certain embodiments, the compositions comprise glycopeptides, including but not limited to trimers as particulate, high-density array on liposomes or other particles, for example but not limited to nanoparticles.

In certain embodiments, where the nucleic acids are operably linked to a promoter and inserted in a vector, the vector is any suitable vector. Non-limiting examples, include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, 3M052, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339).

In certain aspects the invention provides a cell comprising a nucleic acid encoding any one of the glycopeptides of the invention suitable for recombinant expression. In certain aspects, the invention provides a clonally derived population of cells encoding any one of the glycopeptides of the invention suitable for recombinant expression. In certain aspects, the invention provides a sable pool of cells encoding any one of the glycopeptides of the invention suitable for recombinant expression.

### Sequences/Clones

In certain aspects, the invention provides composition and methods which use a selection of glycopeptides as proteins, including glycopeptides displayed on a nanoparticle, DNAs, RNAs, or any combination thereof, administered as primes and boosts to elicit immune response. Glycopeptides as proteins can be co-administered with nucleic acid vectors containing glycopeptides to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, can be administered with each immunization with Env gene inserts in a suitable vector, for example but not limited to HSV-2.

In certain aspects the invention provides compositions and methods of genetic immunization. Nucleotide-based vaccines offer a flexible vector format to immunize against virtually any protein antigen. Currently, two types of genetic vaccination are available for testing—DNAs and mRNAs.

In certain aspects the invention discloses using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLOS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the glycopeptides are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16:319-23, 2010), recombinant mycobacteria (e.g. rBCG or M smegmatis) (Yu, J S et al. Clinical Vaccine Immunol. 14:886-093,2007; ibid 13:1204-11,2006), and recombinant vaccinia type of vectors (Santra S. Nature Med. 16:324-8, 2010), for example but not limited to ALVAC, replicating (Kibler K V et al., PLOS One 6: e25674, 2011 Nov. 9.) and non-replicating (Perreau M et al. J. virology 85:9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention discloses using immunogenic compositions wherein immunogens are delivered as DNA or RNA in suitable formulations. Various technologies which include using DNA or RNA, or can use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp: 293-305 (2012); Arnaoty et al. (2013) Mol Genet Genomics. 2013 August; 288(7-8): 347-63. Nanocarrier technologies called Nanotaxi® for immunogenic macromolecules (DNA, RNA, Protein) delivery are under development. See for example technologies developed by incellart.

In certain aspects the invention discloses using immunogenic compositions wherein immunogens are delivered as recombinant proteins. Various methods for production and purification of recombinant proteins, including glycopeptide ferritin fusion recombinant proteins as described herein, and suitable for use in immunization are known in the art. In certain embodiments recombinant proteins are produced in mammalian cells, e.g. without limitation CHO cells. In certain embodiments, the mammalian cells are GnTI deficient cells.

The envelope glycoproteins or glycopeptides referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204 (1): 266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence can be used. In some embodiments the leader sequence is the endogenous leader sequence. Most of the gp 120 and gp 160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA). Most of the chimeric designs include CD5 leader sequence. A skilled artisan appreciates that when used as immunogens, and for example when recombinantly produced, the amino acid sequences of these proteins do not comprise the leader peptide sequences.

The immunogenic glycopeptides can also be administered as a protein prime and/or boost alone or in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to 3M052, alum, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen [Leroux-Roels et al., IABS Conference, April 2013]. In certain embodiments, TLR agonists are used as adjuvants. In other embodiment, adjuvants which break immune tolerance are included in the immunogenic compositions.

In certain embodiments, the compositions and methods comprise any suitable agent or immune modulation which can modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355 | Foxo1 Inhibitor, AS1842856-Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

Provided are also nucleic acids, including modified mRNAs which are stable and can be used as antigens to induce an immune response. Provided also are nucleic acids optionally designed as vectors, for example for recombinant expression and/or stable integration, e.g. but not limited, DNA encoding protein for stable expression, or VLP incorporation.

In certain aspects, the invention provides nucleic acids comprising sequences encoding proteins of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects the invention provides nucleic acids encoding the inventive protein designs. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

In some embodiments the antibodies are administered as nucleic acids, including but not limited to mRNAs which can be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, US Pub 20180344838A1 at least at paragraphs [0260]-[0281] for non-limiting embodiments of chemical modifications, wherein each content is incorporated by reference in its entirety. In non-limiting embodiments, a modified mRNA comprises pseudouridine. In some embodiments, the modified mRNA comprises 1-methyl-pseudouridine.

mRNAs delivered in LNP formulations have advantages over non-LNPs formulations. See US Pub 20180028645A1.

In certain embodiments the nucleic acid encoding a protein is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequences described herein, or a variant thereof or a fragment thereof. Accordingly, in one embodiment, the invention provides an RNA molecule encoding one or more of inventive antibodies. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7 mG (5′) ppp (5′) NlmpNp). This cap can enhance in vivo translation of the RNA. The 5′ nucleotide of an RNA molecule useful with the invention may have a 5′ triphosphate group. In a capped RNA this may be linked to a 7-methylguanosine via a 5′- to -5′ bridge. An RNA molecule may have a 3′ poly-A tail. It may also include a poly-A polymerase recognition sequence (e.g. AAUAAA) near its 3′ end. In some embodiments, an RNA molecule useful with the invention may be single-stranded. In some embodiments, an RNA molecule useful with the invention may comprise synthetic RNA.

The recombinant nucleic acid sequence can be an optimized nucleic acid sequence. Such optimization can increase or alter the immunogenicity of the protein. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating cis-acting sequence motifs (i.e., internal TATA boxes).

Methods for in vitro transfection of mRNA and detection of protein expression are known in the art.

Methods for expression and immunogenicity determination of nucleic acid encoded proteins are known in the art.

Polynucleotides encoding any of the disclosed immunogens and/or recombinant proteins are also provided. These polynucleotides include DNA, cDNA and RNA sequences which encode the immunogen, as well as vectors including the DNA, cDNA and RNA sequences, such as a DNA or RNA vector used for immunization. The genetic code to construct a variety of functionally equivalent nucleic acids, such as nucleic acids which differ in sequence but which encode the same protein sequence, or encode a conjugate or fusion protein including the nucleic acid sequence.

### Adjuvants

In non-limiting embodiments, compositions can be formulated with appropriate carriers and adjuvants using techniques to yield compositions suitable for immunization. The compositions can include an adjuvant, such as, for example but not limited to alum, 3M052, poly IC, MF-59 or another squalene-based oil-in-water emulsion adjuvant, AS01B, or other liposomal based adjuvant suitable for protein or nucleic acid immunization. In non-limiting embodiments, LNPs are used as adjuvants for immunogenic formulations comprising proteins, including multimeric protein complexes and nanoparticles. In certain embodiments, the adjuvant is GSK AS01E adjuvant containing MPL and QS21. This adjuvant has been shown by GSK to be as potent as the similar adjuvant AS01B but to be less reactogenic. In certain embodiments, AS04, a combination of alum and 3-O-desacyl-4′-monophosphoryl lipid A (MPL) developed by GSK. In certain embodiments the adjuvant is AS03, an oil-in-water emulsion combination adjuvant developed by GSK. Non-limiting embodiments of adjuvants are described in the NIH 2018 Strategic Plan for Research on Vaccine Adjuvants. https://www.niaid.nih.gov/sites/default/files/NIAIDStrategicPlanVaccineAdjuvants2018.pdf

In certain embodiments, the composition and methods comprise an adjuvant. Non-limiting examples of adjuvants include GLA/SE, alum, Poly I poly C (poly IC), polyIC/long chain (LC) TLR agonists, TLR7/8 and/or 9 agonists (e.g. CpG-oligodeoxynucleotide (oCpG)), or a combination of TLR7/8 and TLR9 agonists (see Moody et al. (2014) J. Virol. March 2014 vol. 88 no. 6 3329-3339), or any other suitable adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and CpG-oligodeoxynucleotide (oCpG) in STS (see Moody et al. (2014) J. Virol. March 2014 vol. 88 no. 6 3329-3339).

In certain embodiments, the adjuvant can be Alum (aluminum hydroxide) or variants of Alum such as pSer Alum (Moyer et al. Nat Med 2020 March; 26 (3): 430-440; doi: 10.1038/s41591-020-0753-3. Epub 2020 Feb. 17.).

In certain embodiments, TLR agonists are used as adjuvants. In certain embodiments, TLR agonists are TLR7/8 agonist. Non-limiting examples of TLR 7/8 are described in Evans et al. ACS Omega 2019, 4, 13, 15665-15677; Patinote et al. Eur J Med Chem. 2020 May 1; 193:112238, Miller et al. in Front. Immunol., 10 Mar. 2020| https://doi.org/10.3389/fimmu.2020.00406. In certain embodiments, adjuvants are TLR 4 agonists. In some embodiments, the adjuvants are saponins. See e.g. WO2019079160A1 and references cited therein. In some embodiments, saponins are natural, synthetic, or semi-synthetic saponins.

Non-limiting embodiments of adjuvants include without limitation inulin based adjuvants, see e.g. Advax in Petrovsky and Cooper, Vaccine 2015 Nov. 4; 33 (44): 5920-5926, Advax+TLR agonists and Advax+CpG, see e.g. Counoupas et al. Sci Rep. 2017; 7:8582), saponins, Alhydroxiquim (TLR7/8), IMDQ-Dendrimer (TLR7), Polymeric TLR7/8 adjuvants, Mastoparn-7 (M7), adjuvant LT (R192G/L211A) also referred to as dmLT (see e.g. Celemens and Norton, mSphere. 2018 July-August; 3 (4): e00215-18).

In certain embodiment, adjuvants which break immune tolerance are included in the immunogenic compositions (e.g. Verkoczy et al. J Immunol. 2013 Sep. 1; 191 (5): 2538-50; doi: 10.4049/jimmunol. 1300971. Epub 2013 Aug. 5.).

In non-limiting embodiments, different adjuvants can be combined.

Other non-limiting embodiments of adjuvants that may be used include without limitation Matrix M (e.g. Gorman et al. in bioRxiv. 2021 Feb. 5:2021.02.05.429759. doi: 10.1101/2021.02.05.429759. Preprint. PMID: 33564763), ALFQ from the Military HIV Research Program (e.g. “Army Liposome Formulation (ALF) family of vaccine adjuvants” Alving C R, Peachman K K, Matyas G R, Rao M, Beck Z.Expert Rev Vaccines. 2020 March; 19 (3): 279-292. doi: 10.1080/14760584.2020.1745636. Epub 2020 Mar. 31.PMID: 32228108), MF-59 (e.g. Safety and effectiveness of MF-59 adjuvanted influenza vaccines in children and adults. Black S. Vaccine. 2015 Jun. 8; 33 Suppl 2: B3-5. doi:

10.1016/j.vaccine.2014.11.062.PMID: 26022564), GLA-SE or aqueous formulation (e.g. Felber et al. Cell Rep. 2020 May 12; 31 (6): 107624. doi: 10.1016/j.celrep.2020.107624.PMID: 32402293), CpG 1018 (e.g “Development of CpG-adjuvanted stable prefusion SARS-COV-2 spike antigen as a subunit vaccine against COVID-19” Kuo T Y, Lin M Y, Coffman R L, Campbell J D, Traquina P, Lin Y J, Liu L T, Cheng J, Wu Y C, Wu C C, Tang W H, Huang C G, Tsao K C, Chen C. Sci Rep. 2020 Nov. 18; 10 (1): 20085. doi: 10.1038/s41598-020-77077-z.PMID: 33208827), Adjuplex (e.g. “Carbomer-based adjuvant elicits CD8 T-cell immunity by inducing a distinct metabolic state in cross-presenting dendritic cells.” Lee W, Kingstad-Bakke B, Paulson B, Larsen A, Overmyer K, Marinaik C B, Dulli K, Toy R, Vogel G, Mueller K P, Tweed K, Walsh A J, Russell J, Saha K, Reyes L, Skala M C, Sauer J D, Shayakhmetov D M, Coon J, Roy K, Suresh M.PLOS Pathog. 2021 Jan. 14; 17 (1): e1009168. doi: 10.1371/journal.ppat. 1009168. eCollection 2021 January. PMID: 33444400) and inulin based adjuvants (e.g. “Research Note: The immune enhancement ability of inulin on ptfA gene DNA vaccine of avian Pasteurella multocida.” Gong Q, Peng Y G, Niu M F, Qin C L. Poult Sci. 2020 June; 99 (6): 3015-3019. doi: 10.1016/j.psj.2020.03.006. Epub 2020 Mar. 24.PMID: 32475437).

The content of each and every citation is incorporated by reference in its entirety.

Provided herein are TLR7/8 agonists that can be used in the compositions described herein. As used herein, a “TLR7/8 agonist” refers to an agonist that affects its biological activities through its interaction with TLR7, TLR8, or both Such biological activities include, but are not limited to, the induction of TLR7 and/or TLRS mediated signal transduction to potentiate immune responses via the innate immune system. In some embodiments, the TLR is an imidazoquinoline amine derivative (see. e.g., U.S. Pat. No 4,689,338 (Gerster)), but other compound classes are known as well (see, e.g., U.S. Pat. No. 5,446,153 (Lindstrom et al.); U.S. Pat No. 6,194,425 (Gerster et al), and U.S. Pat. No. 6,110,929 (Gerster et al.); and International Publication Number WO2005/079195 (Hays et al.)).

DNA sequences encoding the disclosed recombinant proteins can be expressed in vitro by DNA transfer into a suitable host cell. The cell may be prokaryotic or eukaryotic. The term also includes any progeny of the subject host cell. All progeny may not be identical to the parental cell since there may be mutations that occur during replication. Methods of stable transfer, meaning that the foreign DNA is continuously maintained in the host, are known in the art.

Host systems for recombinant production can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4.sup.th Ed., Humana Press). Examples of mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI.sup.-/- cells, or HEK-293F cells.

In some embodiments, the disclosed recombinant proteins can be expressed in cells under conditions where the recombinant protein can self-assemble into trimers and/or are secreted from the cells into the cell media. In such embodiments, each recombinant protein contains a leader sequence (signal peptide) that causes the protein to enter the secretory system, where the signal peptide is cleaved, and the protomers form a trimer, before being secreted in the cell media. The medium can be centrifuged and recombinant protein purified from the supernatant.

A nucleic acid molecule encoding an immunogen can be included in a viral vector, for example, for expression of the immunogen in a host cell, or for immunization of a subject as disclosed herein. In some embodiments, the viral vectors are administered to a subject as part of a prime-boost vaccination. In several embodiments, the viral vectors are included in a vaccine, such as a primer vaccine or a booster vaccine for use in a prime-boost vaccination.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

In several embodiments, the viral vector can include an adenoviral vector that expresses an immunogen of the invention. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/02231 1.

In some embodiments, a virus-like particle (VLP) is provided that comprises a recombinant protein of the invention. In some embodiments, a virus-like particle (VLP) is provided that includes a recombinant protein of the invention. Such VLPs can include a recombinant protein that is membrane anchored by a C-terminal transmembrane domain. VLPs lack the viral components that are required for virus replication and thus represent a highly attenuated, replication-incompetent form of a virus. However, the VLP can display a polypeptide that is analogous to that expressed on infectious virus particles and can eliciting an immune response when administered to a subject. Virus like particles and methods of their production are known and familiar to the person of ordinary skill in the art, and viral proteins from several viruses are known to form VLPs, including human papillomavirus, HIV (Kang et al., Biol. Chem. 380:353-64 (1999)), Semliki-Forest virus (Notka et al., Biol. Chem. 380:341-52 (1999)), human polyomavirus (Goldmann et al., J. Virol. 73:4465-9 (1999)), rotavirus (Jiang et al., Vaccine 17:1005-13 (1999)), parvovirus (Casal, Biotechnology and Applied Biochemistry, Vol 29, Part 2, pp 141-150 (1999)), canine parvovirus (Hurtado et al., J. Virol. 70:5422-9 (1996)), hepatitis E virus (Li et al., J. Virol. 71:7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

The immunogens of the invention can be combined with any suitable adjuvant.

In certain embodiments, the immunogens of the invention are designed to form a multimeric complex. In certain embodiments, the proteins are designed to form a multimeric complex. In non-limiting embodiments, the multimeric complexes comprise a ferritin sequence. In non-limiting embodiments, the multimeric complexes comprising a ferritin sequence are designed and are assembled as ferritin fusion proteins. In non-limiting embodiments, the multimeric complexes comprising a ferritin sequence are designed and are assembled via sortase reaction. In non-limiting embodiments, the multimeric complexes comprise encapsulin. These multimeric complexes are nanoparticles.

Multimeric complexes presenting of antigens—nanoparticles. Presenting multiple copies of antigens to B cells has been a longstanding approach to improving B cell receptor recognition and antigen uptake (Batista and Neuberger, 2000). The improved recognition of antigen is due to the avid interaction of multiple antigens with multiple B cell receptors on a single B cells, which results in clustering of B cells and stronger cell signaling. Furthermore, multimeric presentation improves antigen binding to mannose binding lectin which promotes antigen trafficking to B cell follicles (Tokatlian et al., 2019). Self-assembling complexes comprising multiple copies of an antigen are one strategy of immunogen design approach for arraying multiple copies of an antigen for recognition by the B cell receptors on B cells (Kanekiyo et al., 2013; Ueda et al., 2020).

In some instances, the gene of an antigen can be fused via a linker/spacer to a gene of a protein which can self-assemble. Upon translation, a fusion protein is made that can self-assemble into a multimeric complex-also referred to as a nanoparticle displaying multiple copies of the antigen. In other instances, the protein antigen can be conjugated to the self-assembling protein via an enzymatic reaction, thereby forming a nanoparticle displaying multiple copies of the antigen.

Multivalent/multimeric presentation using ferritin. Ferritin is a well-known protein that self-assembles into a hollow particle composed of repeating subunits. In some species ferritin nanoparticles are composed of 24 copies of a single subunit, whereas in other species it is composed of 12 copies each of two subunits. We chose here the two subunit ferritin nanoparticle from Trichoplusia ni since the viral antigen of choice can be fused to, for example, one or both chains of the ferritin nanoparticle. Thus, by fusing the antigen to only one chain the valency can be 12 individual antigens or by fusing the antigen to both chains the valency can be increased to 24 copies of the antigen. While 24 copies of the antigen may increase the interaction with lectins that enhance antigen trafficking, it can also make the nanoparticle unstable or preclude access to epitopes on the sides of the exogenous antigen because the antigens are too densely clustered on the nanoparticle. In such instances having the ability to form nanoparticles with lower antigen valency because the antigen is fused to only one ferritin chain will be beneficial.

Structural considerations and ferritin selection in design of nanoparticle. Previous studies have attempted to use ferritin to display complex antigens on their surface. However, the fusion of heterologous proteins to ferritin and the formation of nanoparticles is inconsistent. Often the nanoparticle fails to form or does so inefficiently resulting in low yields. This necessitates many rounds of iterative protein design to find the optimal fusion protein construct. We inspected the structure of Helicobacter pylori ferritin to determine reasons why fusing the gene of a protein of interest to the 5′ end of the ferritin gene might disrupt nanoparticle formation upon translation. Without being bound by theory, several considerations are discussed. First, the N-terminus of Helicobacter pylori ferritin subunits adopt an alpha helix structure. The fusion of the exogenous protein must occur at the point in the sequence where the helix has turned away from the center of the nanoparticle. Therefore, the N-terminus can be truncated to the Asp residue at position 5 since it is the last apical amino acid. Second, the sequence of the heterologous fused protein will have to be compatible with the alpha helix secondary structure. The heterologous fused protein cannot break the alpha helix secondary structure or the ferritin subunit will not properly fold. At the same time, the sequence of heterologous fused protein cannot continue the alpha helix structure or the extended helix will clash with adjacent ferritin chains. To disrupt the alpha helix we have designed long linkers, which can be flanked by prolines (e.g. PGS19 and PGS34) to break the secondary structure of the peptide to which the heterologous protein is attached. In some embodiments, linkers for use in any of the designs of the invention can be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine. Third, the tertiary structure of each H. pylori ferritin subunit causes the N-terminal helix to be distal to the three-fold symmetrical axis in the center of the trimeric subunits. Thus, the interaction or clustering of the heterologous antigens requires crossing over top of two or more ferritin subunit alpha helices. The heterologous protein has to attach to the ferritin subunit in such a way that it can turn the peptide chain to the left to cross over top of the helices. Such a redirection of the peptide chain can only happen if the heterologous protein does not continue the alpha helical secondary structure of the N-terminus of H. pylori ferritin.

Trichoplusia ni (T. ni) ferritin is an alternative sequence to the H. pylori ferritin. T. ni has two ferritin chains whose structures are different from Helicobacter pylori ferritin. In contrast to H. pylori ferritin chains, the two chains of T. ni ferritin have long flexible N-terminal regions. The limited intramolecular interactions of these N-terminus with other ferritin chains allows it to be easily fused to a heterologous protein without disrupting the overall ferritin chain structure or assembly of the particle. Without being bound by theory fusion proteins where an antigen sequence is fused to T. ni ferritin sequence overcomes some of the technical issues when H. pylori ferritin is used.

Increasing the valency of self-assembling nanoparticles. Encapsulin nanoparticles have 60 or more subunits to which heterologous proteins can attach. This provides an increase of 36 copies of a heterologous antigen of choice compared to ferritin nanoparticles. Encapsulins also have the advantage of being able to encapsulate other proteins inside the nanoparticle (Sutter et al., 2008). Structures of Thermotoga maritima, Myxococcus xanthus, and Pyrococcus furiosus encapsulins have been solved, which allows for determination of the sites of attachment (Gabashvili et al., 2020). The structure of Thermotoga maritima encapsulin showed that it had 60 repeat subunits that comprised one nanoparticle (Sutter et al., 2008). Some encapsulin nanoparticles such as Pdu nanoparticles are composed of three or more subunits making it more complicated to express and form as an intact nanoparticle (Crowley et al., 2010; Havemann et al., 2002). The N-terminus of T. maritima encapsulin points into the lumen of the particle whereas the C-terminus points out away from the nanoparticle. Thus, the C-terminus of Thermotoga maritima encapsulin is the optimal attachment site for a heterologous protein so that the folding of the encapsulin subunit or the assembly of the nanoparticle in not disrupted. The C-terminus is also an optimal site for attachment since it lacks secondary structure and is just a flexible loop pointing away from the nanoparticle. Encapsulin nanoparticles displaying Epstein-Bar virus glycoproteins have been successfully achieved validating their use as a nanoparticle scaffold (Kanekiyo et al., 2015).

Nucleic vaccines are an attractive platform since they can be rapidly manufactured and vaccination with them can elicit lasting humoral immunity (Pardi et al., 2018; Saunders et al., 2020). Since nucleic acid vaccines express the protein in vivo the quality of the immunogen is controlled by designing a gene that results in properly-folded protein. Without wishing to be bound by theory, the designs put forward here can fold properly making them suitable for nucleic vaccine platforms such as lipid nanoparticle encapsulated mRNA or DNA electroporation.

Presentation of antigens as particulates reduces the B cell receptor affinity necessary for signal transduction and expansion (See Batista et al. EMBO J. 2000 Feb. 15; 19 (4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. The stronger B cell receptor interaction leads to stronger B cell activation and proliferation. Provided are proteins or portions thereof as particulate, high-density array on liposomes or other particles, for example but not limited to nanoparticles. See e.g. He et al. Nature Communications 7, Article number: 12041 (2016), doi: 10.1038/ncomms12041; Bamrungsap et al. Nanomedicine, 2012, 7 (8), 1253-1271.

To improve the interaction between the naïve B cell receptor and immunogens, protein designs can be created to wherein the protein is presented on particles, e.g. but not limited to nanoparticle. Ferritin protein self assembles into a small nanoparticle with three fold axis of symmetry. At these axes the protein is fused. Therefore, the assembly of the three-fold axis also clusters three proteins together. Each ferritin particle has 8 axes which equates to 8 proteins being displayed per particle. See e.g. Sliepen et al. Retrovirology201512: 82, DOI: 10.1186/s12977-015-0210-4.

Any suitable ferritin sequence can be used. In non-limiting embodiments, ferritin sequences are disclosed in WO/2018/005558. In some embodiments, the ferritin sequence comprises N19Q amino acid change.

Ferritin nanoparticle linkers: The ability to form protein ferritin nanoparticles relies on self-assembly of 24 ferritin subunits into a single ferritin nanoparticle. The addition of a ferritin subunit to the C-terminus of a protein or domains thereof may interfere with the ability of the ferritin subunit to fold properly and or associate with other ferritin subunits. When expressed alone ferritin readily forms 24-subunit nanoparticles, however appending it to certain proteins has only yielded low titers of nanoparticles if any. Since the ferritin nanoparticle forms in the absence of these protein, it indicates that there can be steric hindering of the association of ferritin subunits. Thus, we designed ferritin with elongated glycine-serine linkers to further distance the protein from the ferritin subunit. To make sure that the glycine linker is attached to ferritin at the correct position, we created constructs that attach at second amino acid position or the fifth amino acid position. The first four n-terminal amino acids of natural Helicobacter pylori ferritin are not needed for nanoparticle formation but may be critical for proper folding and oligomerization when appended to protein. Thus, we designed constructs with and without the Leucine, serine, and lysine amino acids following the glycine-serine linker. The goal will be to find a linker length that is suitable for formation of protein nanoparticles when ferritin is appended to most proteins. Any suitable linker between the protein and ferritin can be used, so long as the fusion protein is expressed and the nanoparticle is formed.

Another approach to multimerize expression constructs uses staphylococcus Sortase A transpeptidase ligation to conjugate inventive protein immunogens, for e.g. but not limited to cholesterol. The proteins can then be embedded into liposomes via the conjugated cholesterol.

To conjugate a protein of the invention, a C-terminal LPXTG tag or a N-terminal pentaglycine repeat tag is added to the protein immunogen and/or its encoding gene, where X signifies any amino acid, for example Ala, Ser, Glu. Cholesterol is also synthesized with these two tags. Sortase A is then used to covalently bond the tagged protein to the cholesterol.

The sortase A-tagged protein or portion thereof can also be used to conjugate the protein to other peptides, proteins, or fluorescent labels. In non-limiting embodiments, the sortase A tagged proteins or portions are conjugated to ferritin to form nanoparticles.

The invention provides design of various immunogens wherein the immunogen comprises a linker which permits addition of a lipid, such as but not limited to cholesterol, or multimerizing protein via a Sortase A reaction. See e.g. Tsukiji, S. and Nagamune, T. (2009), Sortase-Mediated Ligation: A Gift from Gram-Positive Bacteria to Protein Engineering. ChemBioChem, 10:787-798. doi: 10.1002/cbic.200800724; Proft, T. Sortase-mediated protein ligation: an emerging biotechnology tool for protein modification and immobilisation. Biotechnol Lett (2010) 32:1. doi: 10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35 (8): 4411-7; Pritz et al. J. Org. Chem. 2007, 72, 3909-3912.

### References

Batista, F. D., and Neuberger, M. S. (2000). B cells extract and present immobilized antigen: implications for affinity discrimination. EMBO J 19, 513-520.

Crowley, C. S., Cascio, D., Sawaya, M. R., Kopstein, J. S., Bobik, T. A., and Yeates, T. O. (2010). Structural insight into the mechanisms of transport across the Salmonella enterica Pdu microcompartment shell. J Biol Chem 285, 37838-37846.

Gabashvili, A. N., Chmelyuk, N. S., Efremova, M. V., Malinovskaya, J. A., Semkina, A. S., and Abakumov, M. A. (2020). Encapsulins-Bacterial Protein Nanocompartments: Structure, Properties, and Application. Biomolecules 10.

Havemann, G. D., Sampson, E. M., and Bobik, T. A. (2002). PduA is a shell protein of polyhedral organelles involved in coenzyme B (12)-dependent degradation of 1,2-propanediol in Salmonella enterica serovar typhimurium LT2. J Bacteriol 184, 1253-1261.

Kanekiyo, M., Bu, W., Joyce, M. G., Meng, G., Whittle, J. R., Baxa, U., Yamamoto, T., Narpala, S., Todd, J. P., Rao, S. S., et al. (2015). Rational Design of an Epstein-Barr Virus Vaccine Targeting the Receptor-Binding Site. Cell 162, 1090-1100.

Kanekiyo, M., Wei, C. J., Yassine, H. M., McTamney, P. M., Boyington, J. C., Whittle, J. R., Rao, S. S., Kong, W. P., Wang, L., and Nabel, G. J. (2013). Self-assembling influenza nanoparticle vaccines elicit broadly neutralizing H1N1 antibodies. Nature 499, 102-106.

Kong, R., Xu, K., Zhou, T., Acharya, P., Lemmin, T., Liu, K., Ozorowski, G., Soto, C., Taft, J. D., Bailer, R. T., et al. (2016). Fusion peptide of HIV-1 as a site of vulnerability to neutralizing antibody. Science 352, 828-833.

Li, D., Edwards, R. J., Manne, K., Martinez, D. R., Schafer, A., Alam, S. M., Wiehe, K., Lu, X., Parks, R., Sutherland, L. L., et al. (2021). The functions of SARS-COV-2 neutralizing and infection-enhancing antibodies in vitro and in mice and nonhuman primates. bioRxiv.

Liu, L., Wang, P., Nair, M. S., Yu, J., Rapp, M., Wang, Q., Luo, Y., Chan, J. F., Sahi, V., Figueroa, A., et al. (2020). Potent neutralizing antibodies against multiple epitopes on SARS-COV-2 spike. Nature 584, 450-456.

Pardi, N., Hogan, M. J., Naradikian, M. S., Parkhouse, K., Cain, D. W., Jones, L., Moody, M. A., Verkerke, H. P., Myles, A., Willis, E., et al. (2018). Nucleoside-modified mRNA vaccines induce potent T follicular helper and germinal center B cell responses. J Exp Med 215, 1571-1588.

Piccoli, L., Park, Y. J., Tortorici, M. A., Czudnochowski, N., Walls, A. C., Beltramello, M., Silacci-Fregni, C., Pinto, D., Rosen, L. E., Bowen, J. E., et al. (2020). Mapping Neutralizing and Immunodominant Sites on the SARS-COV-2 Spike Receptor-Binding Domain by Structure-Guided High-Resolution Serology. Cell 183, 1024-1042 e1021.

Saunders, K. O., Pardi, N., Parks, R., Santra, S., Mu, Z., Sutherland, L., Scearce, R., Barr, M., Eaton, A., Hernandez, G., et al. (2020). Lipid nanoparticle encapsulated nucleoside-modified mRNA vaccines elicit polyfunctional HIV-1 antibodies comparable to proteins in nonhuman primates. bioRxiv.

Sutter, M., Boehringer, D., Gutmann, S., Gunther, S., Prangishvili, D., Loessner, M. J., Stetter, K. O., Weber-Ban, E., and Ban, N. (2008). Structural basis of enzyme encapsulation into a bacterial nanocompartment. Nat Struct Mol Biol 15, 939-947.

Tokatlian, T., Read, B. J., Jones, C. A., Kulp, D. W., Menis, S., Chang, J. Y. H., Steichen, J. M., Kumari, S., Allen, J. D., Dane, E. L., et al. (2019). Innate immune recognition of glycans targets HIV nanoparticle immunogens to germinal centers. Science 363, 649-654.

Ueda, G., Antanasijevic, A., Fallas, J. A., Sheffler, W., Copps, J., Ellis, D., Hutchinson, G. B., Moyer, A., Yasmeen, A., Tsybovsky, Y., et al. (2020). Tailored design of protein nanoparticle scaffolds for multivalent presentation of viral glycoprotein antigens. Elife 9.

Wec, A. Z., Wrapp, D., Herbert, A. S., Maurer, D. P., Haslwanter, D., Sakharkar, M., Jangra, R. K., Dieterle, M. E., Lilov, A., Huang, D., et al. (2020). Broad neutralization of SARS-related viruses by human monoclonal antibodies. Science 369, 731-736.

TABLE 1
Non-limiting embodiments of various linkers

| C-terminal Linker/sortase tag fused to | |
|---|---|
| protein or portion thereof before sortase | |
| mediated conjugation | |
| These are short linkers to allow better | |
| access to the sortase A acceptor peptide. | |
| The design goal is to extend the sortase | Linker between protein or portion |
| A tag away from the protein so that it can | thereof and multimerizing protein |
| connect to the nanoparticle with | (e.g. ferritin) after sortase |
| sterically hitting the protein beside it. | conjugation |
| LPXTG(GG) | LPXTGGGGGGSG (X can be E or S) |
| LPXTGG(G) | Or |
| LPXTGGG | LPXTGGGGG (X can be E or S) |
| (G) and (GG) are optional | |
| GSGLPXTG(GG) | GSGLPXTGGGGG (X can be E or |
| GSGLPXTGG(G) | S) |
| GSGLPXTGGG | |
| (G) and (GG) are optional | |
| Linkers in direct fusions between protein | |
| or portion thereof and multimerizing | |
| protein (e.g. ferritin) | |
| [GGS](n) | Embodiment of a flexible linker |
| | Where n = 1, 2,3, 4, 5, 6-10. |
| EKAAKAEEAAR | Embodiments of rigid linkers |
| EKAAKAEEAARP | |
| EKAAKAEEAARPP | |
| GGSEKAAKAEEAAR | Embodiments of flexible and rigid |
| GGSEKAAKAEEAARP | linkers |
| GGSEKAAKAEEAARPP | |

In certain aspects the invention provides a method of producing a multimeric protein complex of the invention the method comprising: expressing a nucleic acid molecule or vector encoding a recombinant fusion protein in a host cell under conditions suitable to produce the recombinant fusion protein; purifying the recombinant fusion protein from the host cell by any suitable purification methods; contacting/reacting the recombinant fusion protein with a self-assembling ferritin protein in the presence of a sortase enzyme in a conjugation reaction, under suitable conjugation conditions to form by sortase conjugation a multimeric ferritin protein complex which comprises the recombinant fusion protein; and isolating the multimeric ferritin protein complex conjugated to the recombinant fusion protein from the rest of the conjugation reaction, which includes unreacted recombinant fusion protein, self-assembling ferritin protein, multimeric ferritin protein complex unconjugated to the recombinant fusion protein and the sortase enzyme. In some embodiments, the self-assembling ferritin protein is formed in a multimeric protein complex prior to contacting with the recombinant fusion protein.

Multimeric glycopeptide designs. Presentation of antigens as particulates reduces the B cell receptor affinity necessary for signal transduction and expansion (See Baptista et al. EMBO J. 2000 Feb. 15; 19 (4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. Very few naïve B cells from which HIV-1 broadly neutralizing antibodies arise can bind to soluble HIV-1 Envelope. Provided are glycopeptides as particulate, high-density array on liposomes or other particles, for example but not limited to nanoparticles. See e.g. He et al. Nature Communications 7, Article number: 12041 (2016), doi: 10.1038/ncomms12041; Bamrungsap et al. Nanomedicine, 2012, 7 (8), 1253-1271.

To improve the interaction between the naïve B cell receptor and immunogens, glycopeptide designed can be created to wherein the glycopeptide is presented on particles, e.g. but not limited to nanoparticle. In some embodiments, the HIV-1 glycopeptide can be fused to ferritin. Ferritin protein self assembles into a small nanoparticle with three fold axis of symmetry. At this axis the envelope glycopeptide is fused. Therefore, the assembly of the three-fold axis also clusters three HIV-1 envelope glycopeptide together to form an envelope trimer. Each ferritin particle has 8 axises. See e.g. Sliepen et al. Retrovirology201512: 82, DOI: 10.1186/s12977-015-0210-4.

Ferritin nanoparticle linkers: The ability to form HIV-1 glycopeptide ferritin nanoparticles relies on self-assembly of 24 ferritin subunits into a single ferritin nanoparticle. The addition of a ferritin subunit to the c-terminus of HIV-1 envelope can interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope can be sterically hindering the association of ferritin subunits. Thus, we designed ferritin with elongated glycine-serine linkers to further distance the envelope from the ferritin subunit. To make sure that the glycine linker is attached to ferritin at the correct position, we created constructs that attach at second amino acid position or the fifth amino acid position. The first four n-terminal amino acids of natural Helicobacter pylori ferritin are not needed for nanoparticle formation, but can be critical for proper folding and oligomerization when appended to envelope. Thus, we designed constructs with and without the Leucine, serine, and lysine amino acids following the glycine-serine linker. The goal is to find a linker length that is suitable for formation of envelope nanoparticles when ferritin is appended to most envelopes.

Another approach to multimerize expression constructs uses staphylococcus Sortase A transpeptidase ligation to conjugate inventive envelope glycopeptides to cholesterol. The glycopeptide can then be embedded into liposomes via the conjugated cholesterol. To conjugate the trimer to cholesterol a C-terminal LPXTG tag or a N-terminal pentaglycine repeat tag is added to the envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles.

The invention provides design of glycoproteins and trimer designs wherein the envelope comprises a linker which permits addition of a lipid, such as but not limited to cholesterol, via a Sortase A reaction. See e.g. Tsukiji, S. and Nagamune, T. (2009), Sortase-Mediated Ligation: A Gift from Gram-Positive Bacteria to Protein Engineering. ChemBioChem, 10:787-798. doi: 10.1002/cbic.200800724; Proft, T. Sortase-mediated protein ligation: an emerging biotechnology tool for protein modification and immobilisation. Biotechnol Lett (2010) 32:1. doi: 10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35 (8): 4411-7; Pritz et al. J. Org. Chem. 2007, 72, 3909-3912.

The lipid modified glycopeptides can be formulated as liposomes. Any suitable liposome composition is disclosed.

Additional sortase linkers can be used so long as their position allows multimerization of the glycopeptides.

### EXAMPLES

### Example 1: Immunofocusing HIV-1 Envelope Glycopeptide Nanoparticle Designs and Their Uses

Previous animal studies have shown administration of monomeric, soluble glycopeptides to be poorly immunogenic, even after multiple doses. In order to elicit more robust responses, we used ferritin nanoparticles to multimerize our designs. Briefly, fusion genes encoding our minimal immunogen designs are cloned into expression vectors and transfected into human cell lines. Nanoparticles spontaneously assemble in vivo and incorporate up to 24 copies of glycopeptide on the surface, per particle. The assembled nanoparticles are secreted into cell supernatant

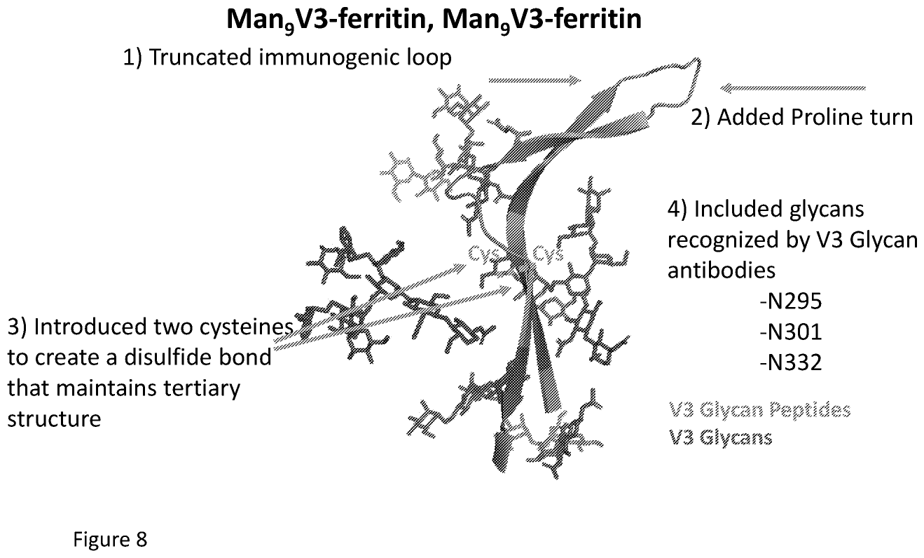

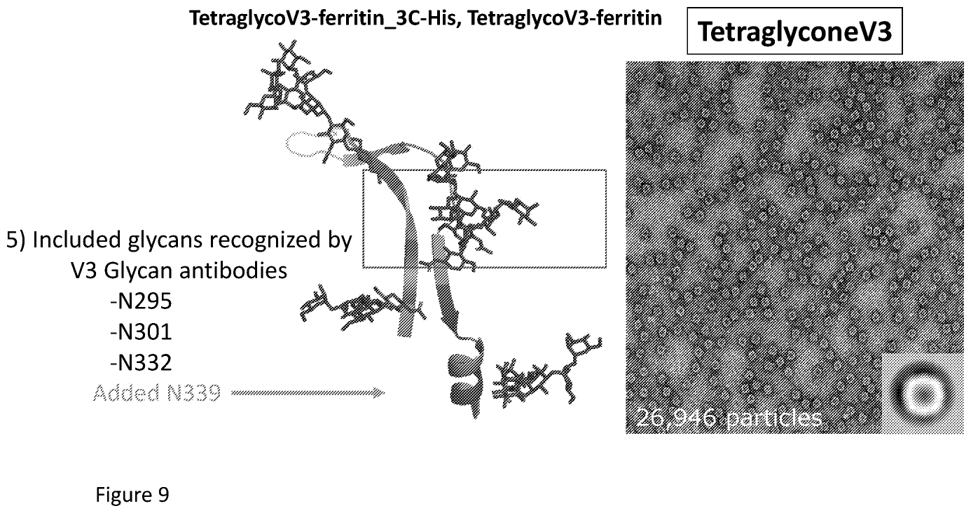

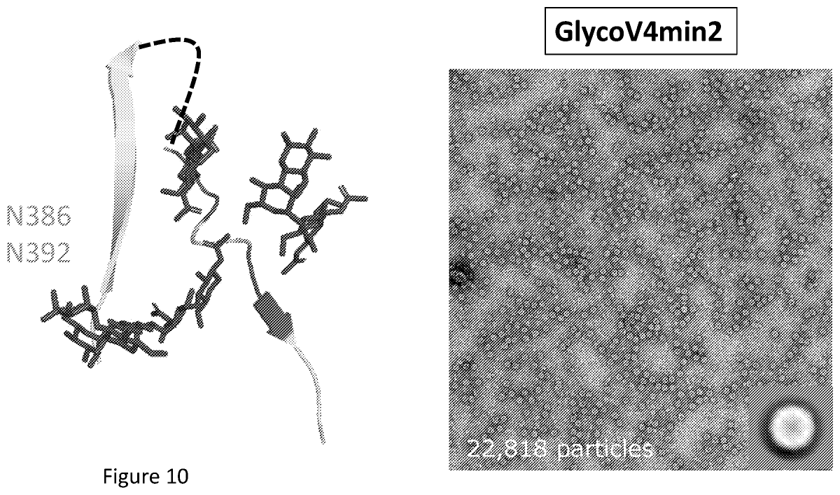

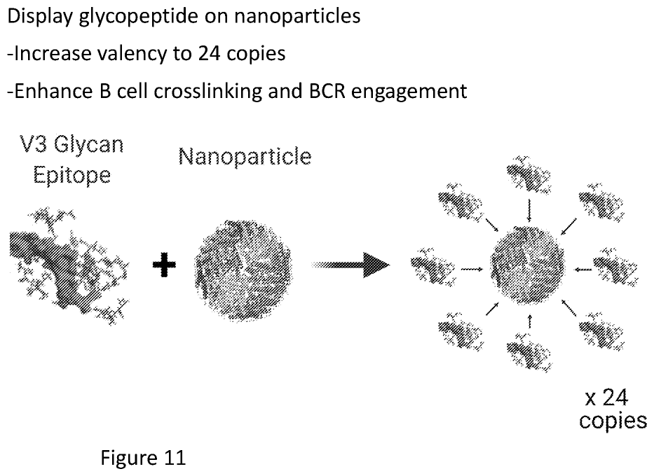

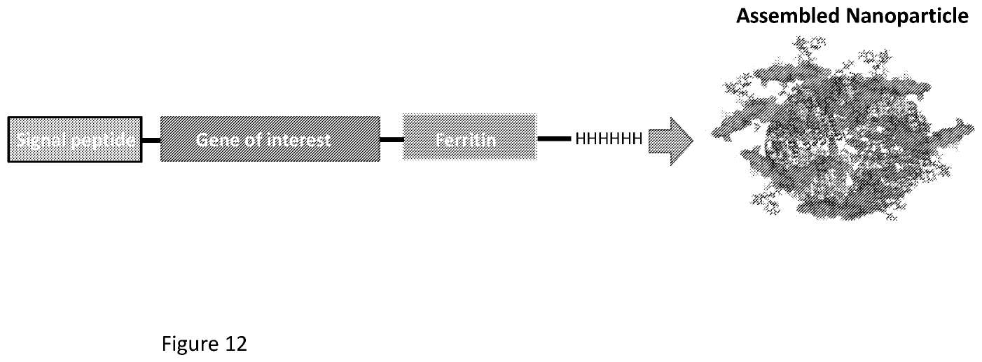

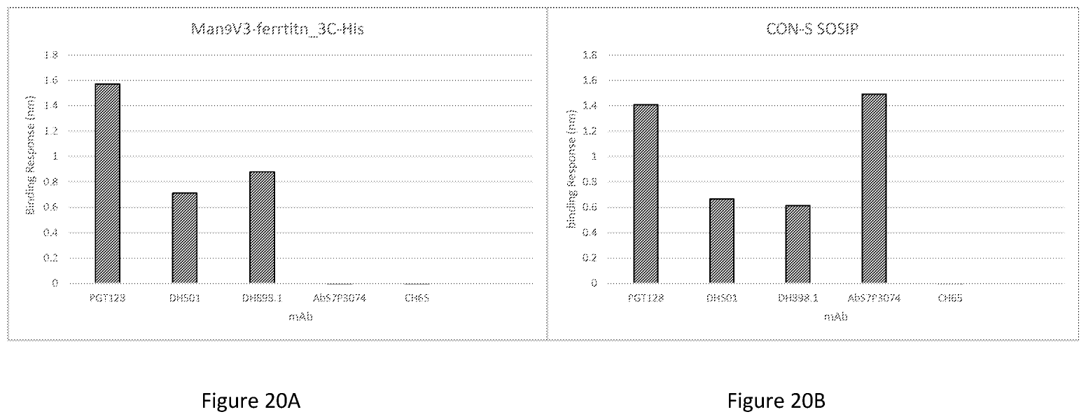

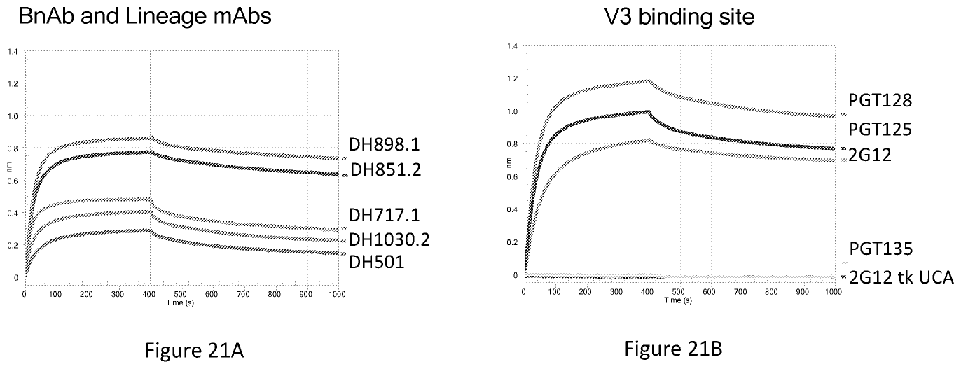

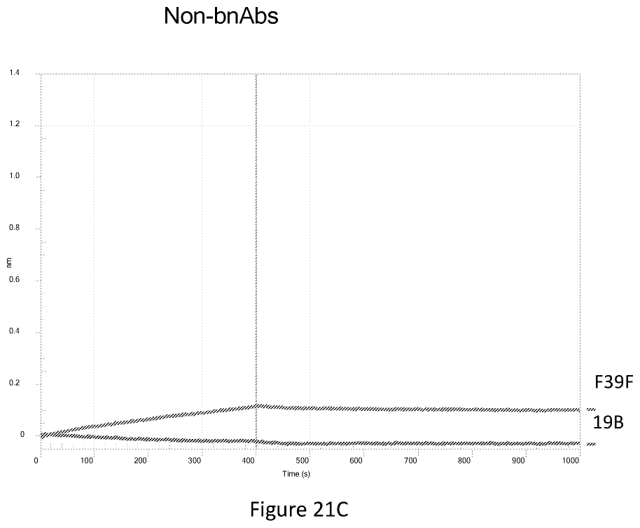

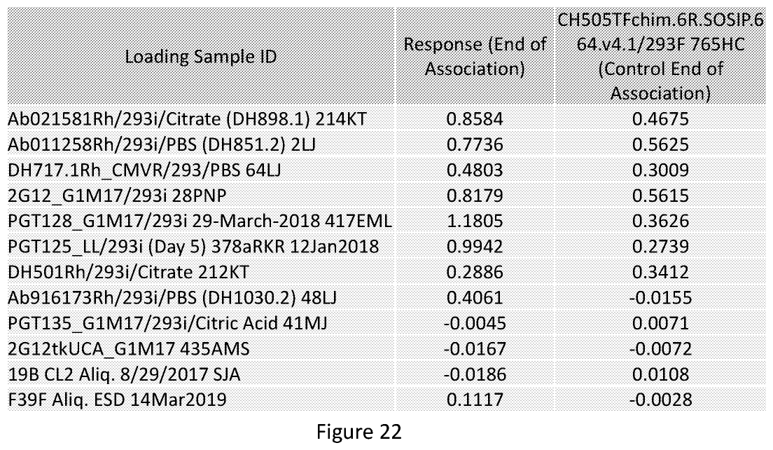

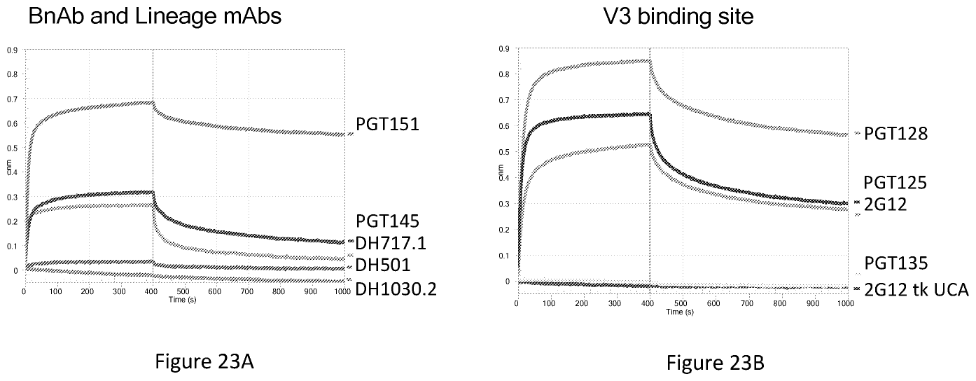

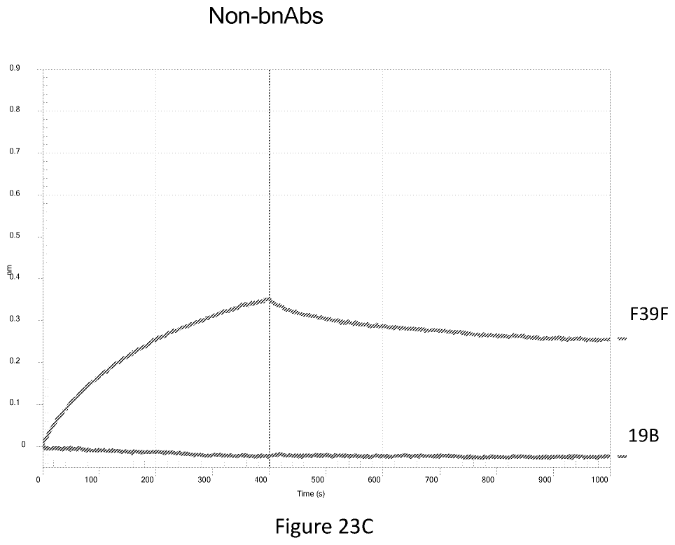

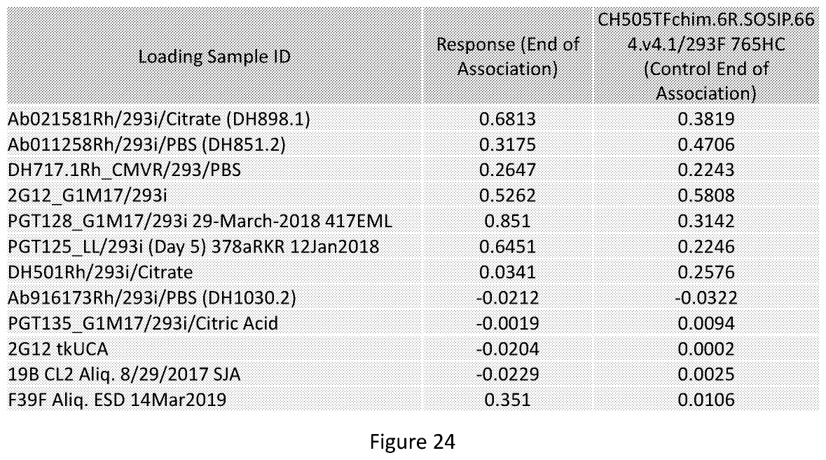

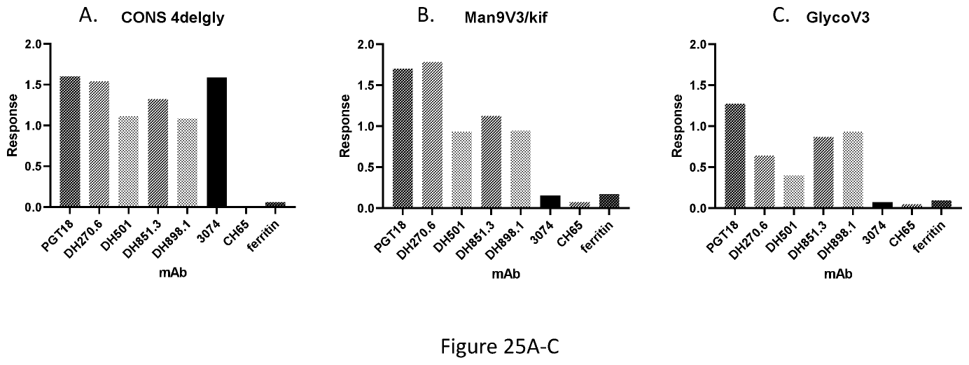

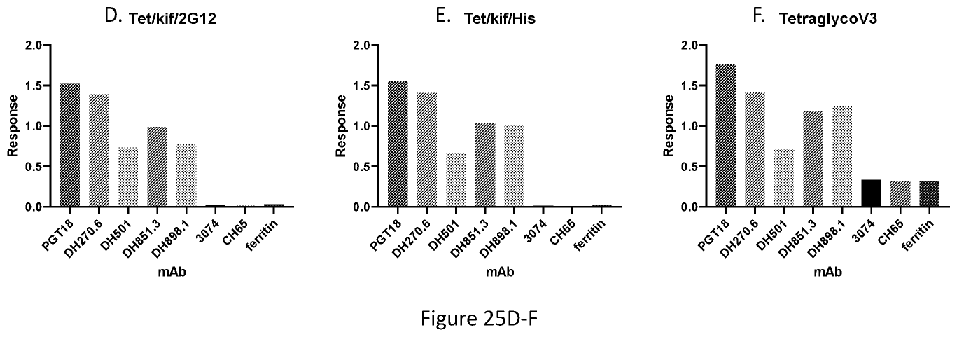

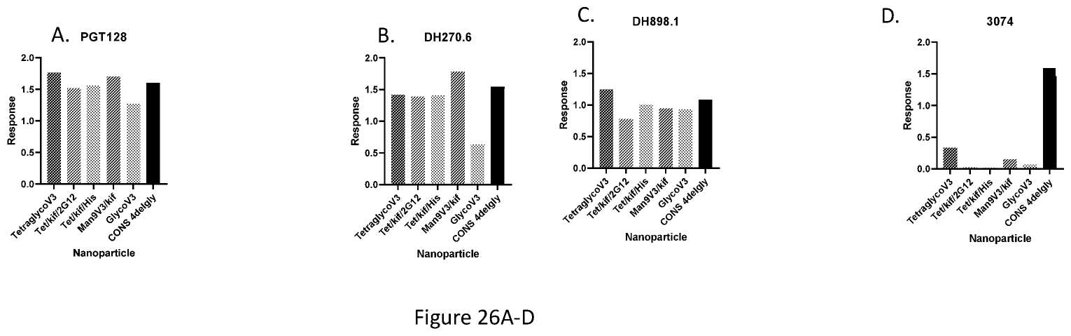

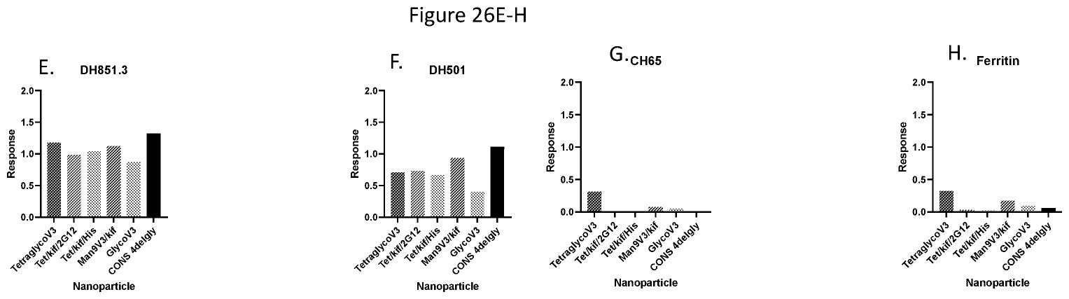

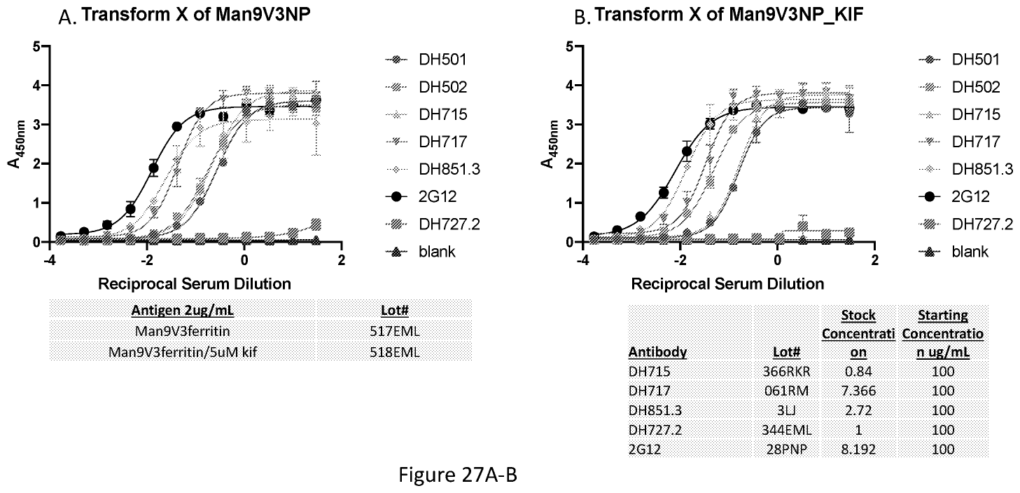

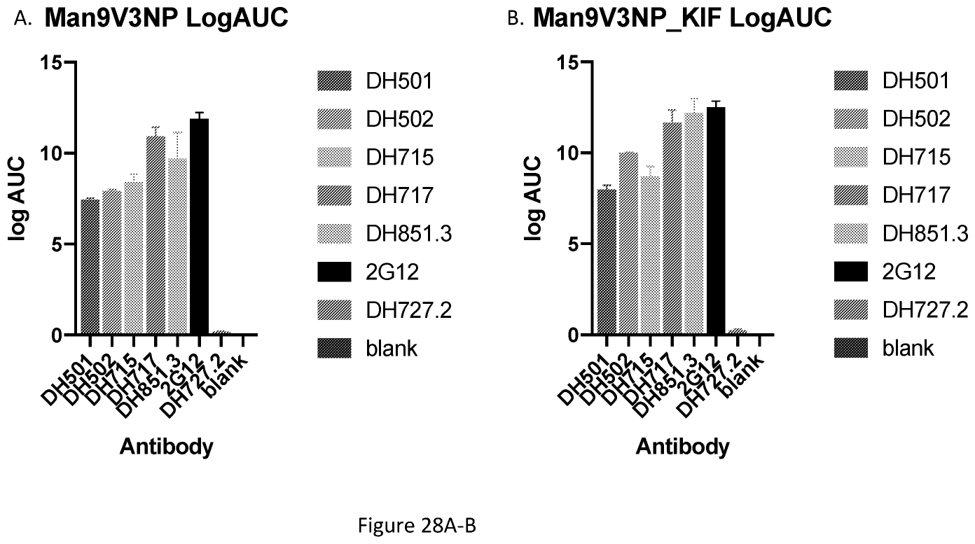

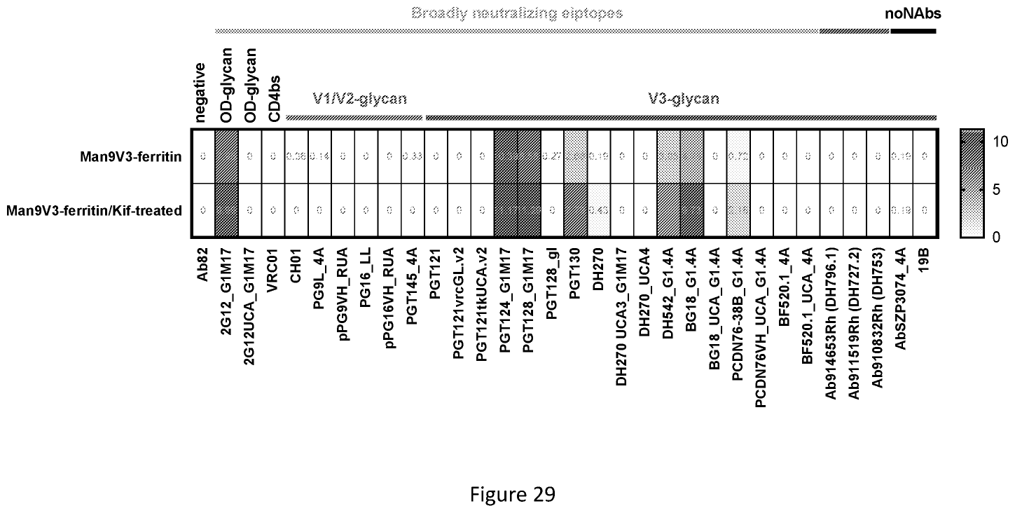

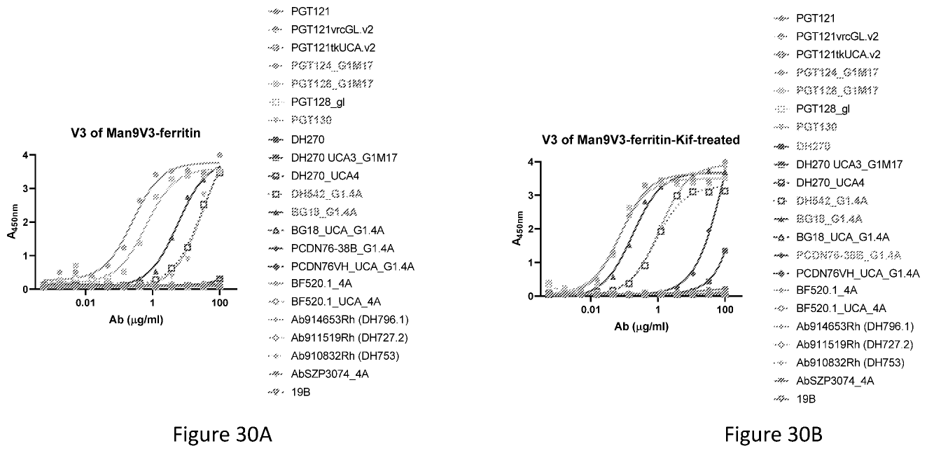

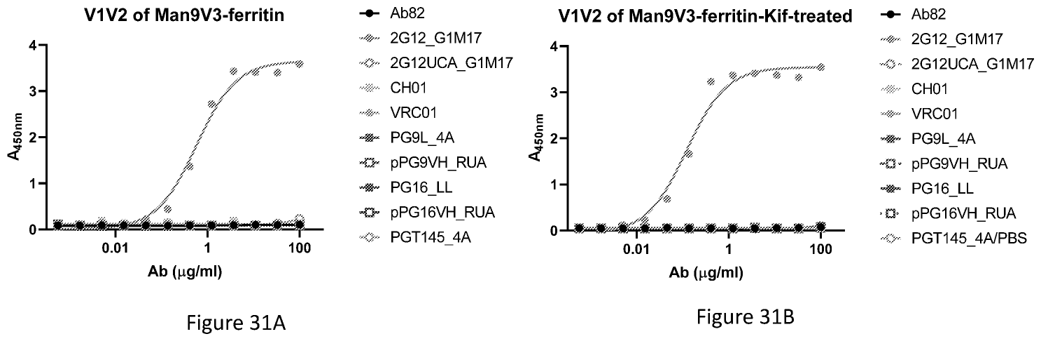

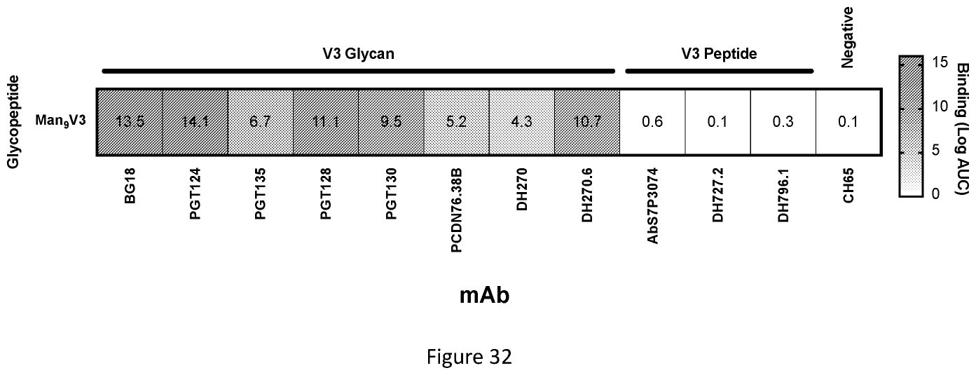

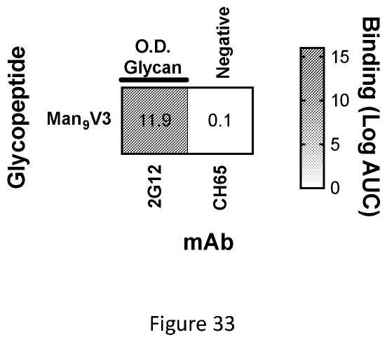

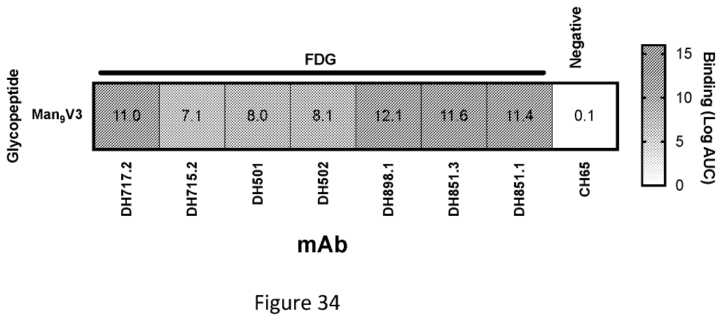

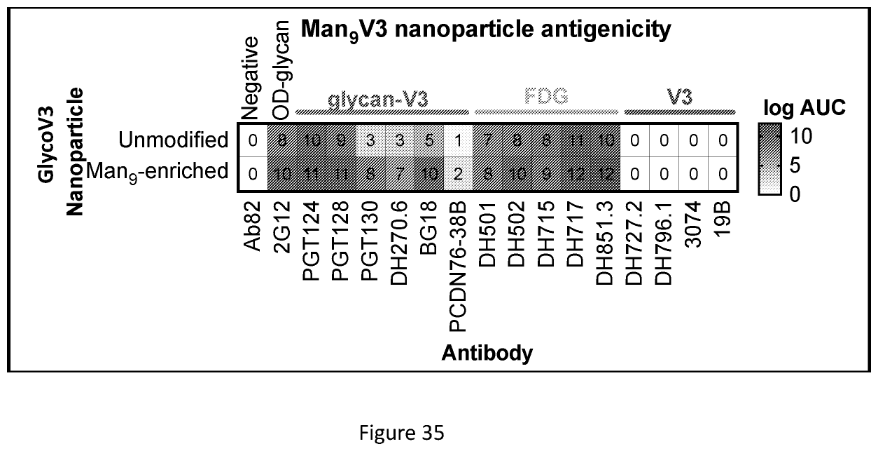

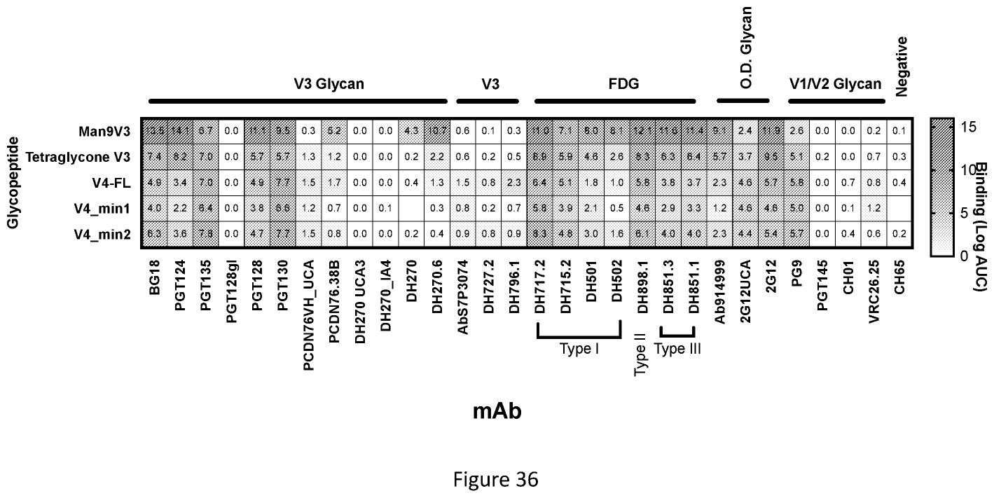

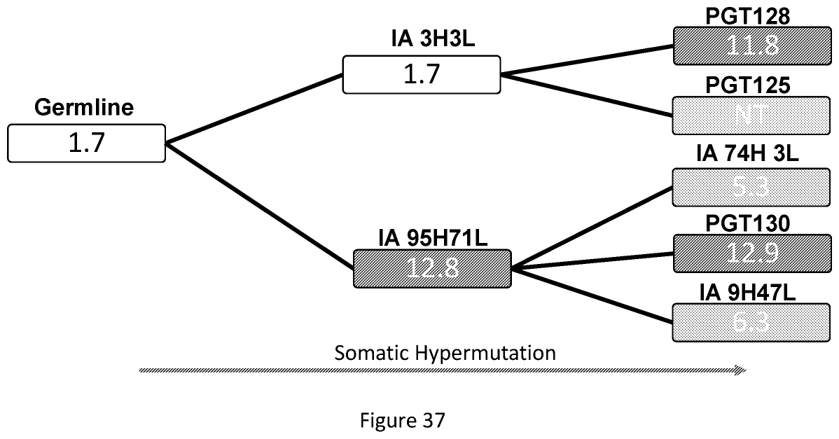

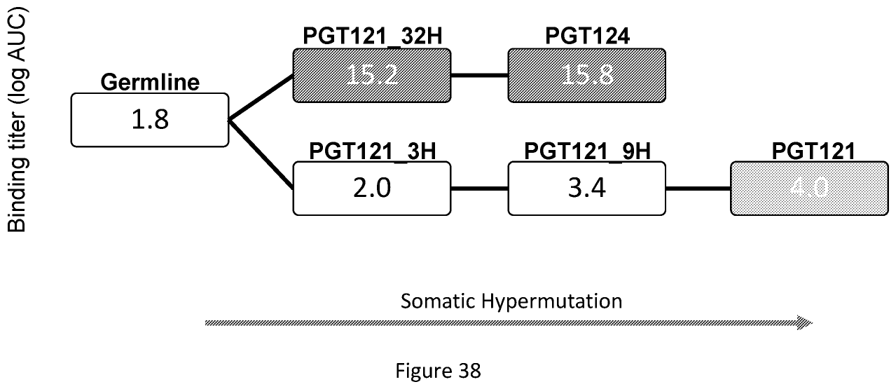

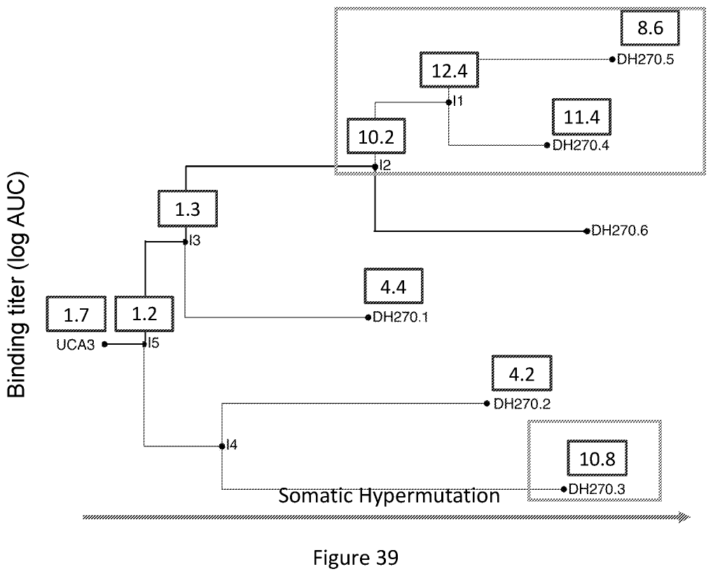

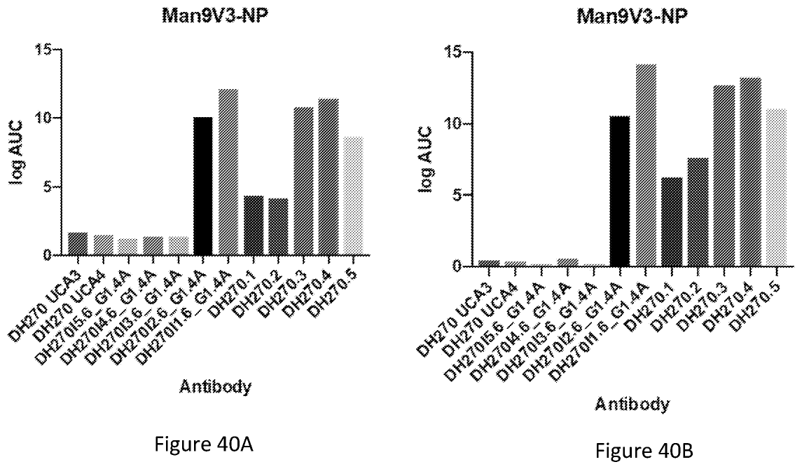

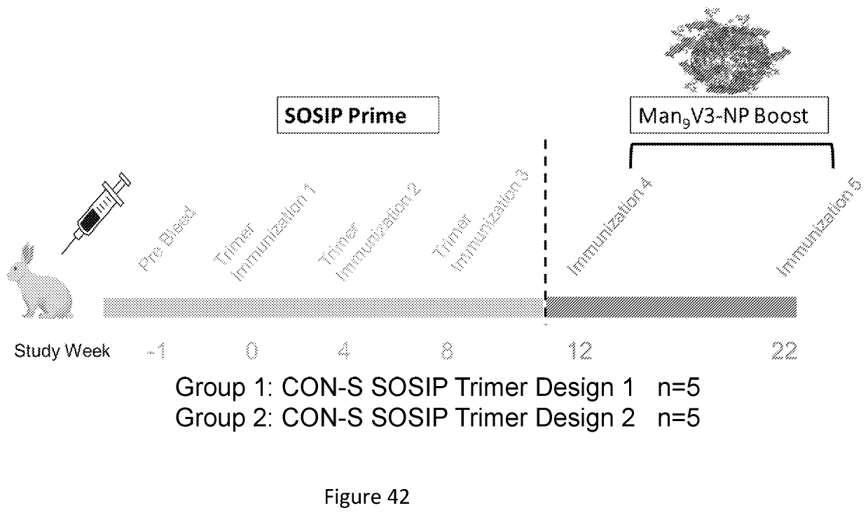

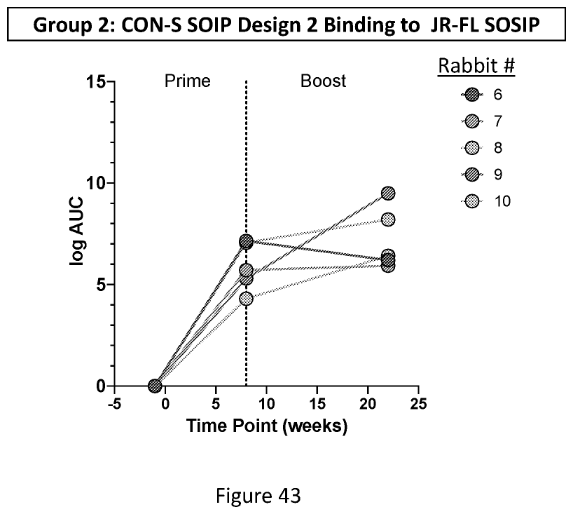

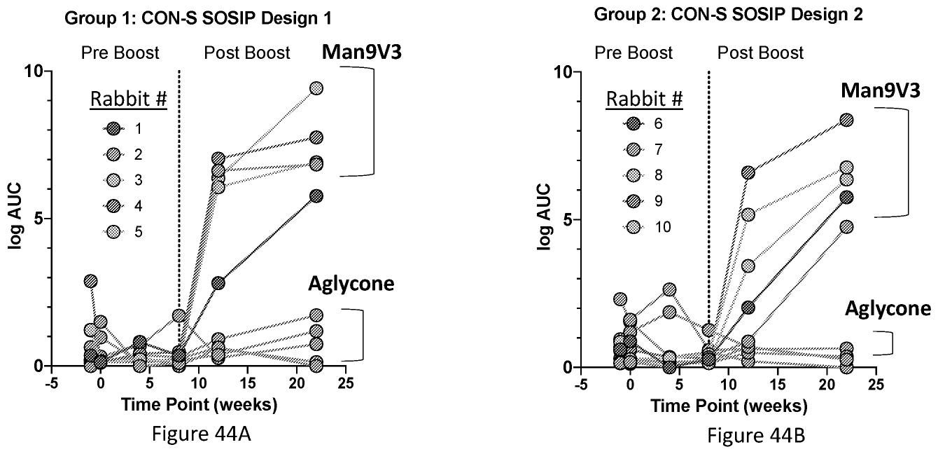

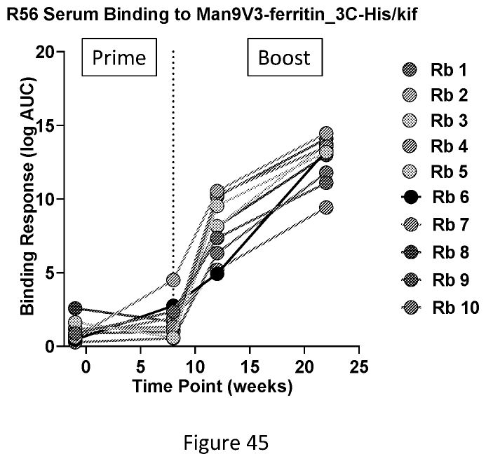

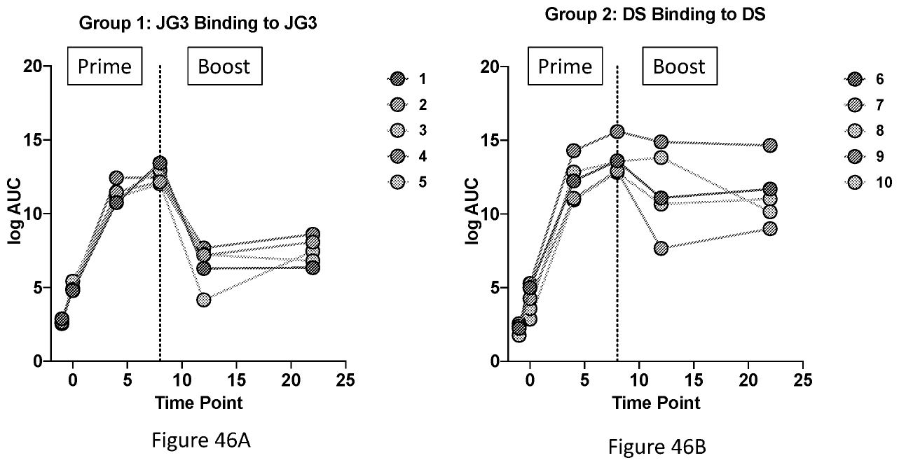

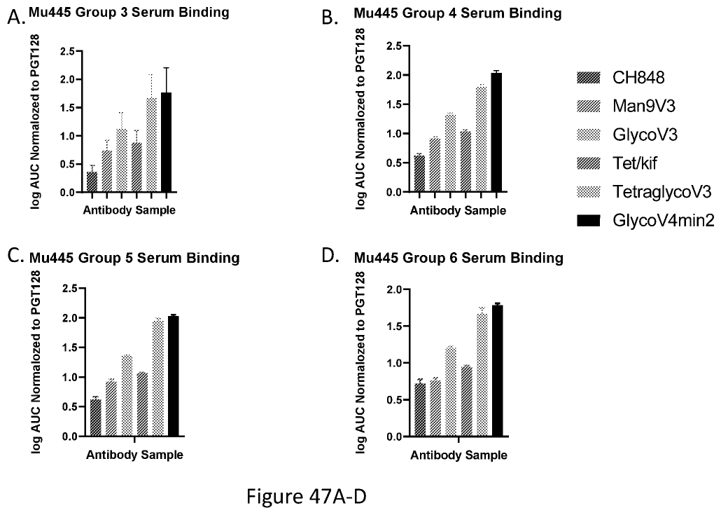

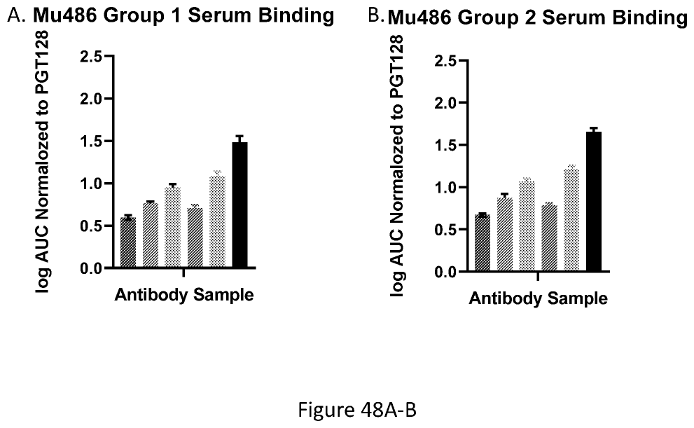

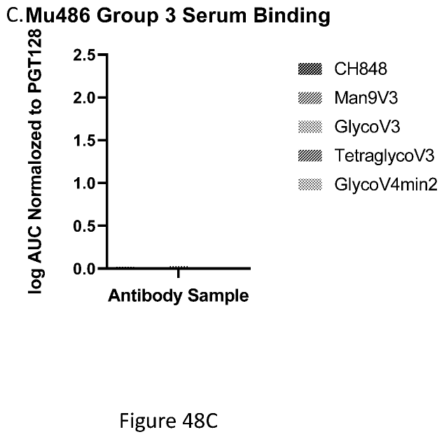

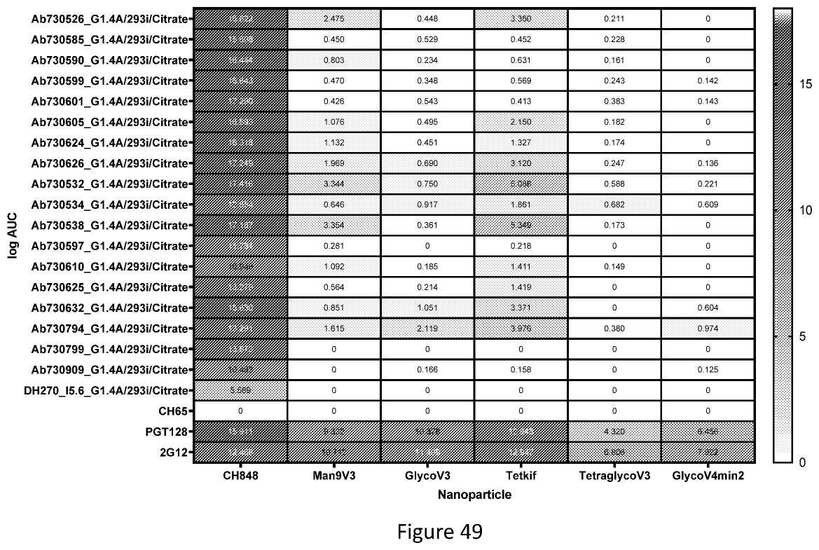

Design of Glycopeptide Nanoparticles and their characterization are shown in FIGS. 8-49.

Multimeric glycopeptide immunogen (FIGS. 8-12). To recapitulate the V3-glycan site we derived 3 glycopeptide antigens composed of amino acids 293 through 304 (EINCTRPNNNTR) and 320 through 336 (GEIIGDIRQAHCNISRA). The immunodominant, strain-specific amino acids 305-319 are replaced in the design by a proline in order to eliminate strain-specific poorly neutralizing antibody responses and to focus the immune response to the conserved amino acids N-terminal and C-terminal to amino acids 305-319. The peptide includes two cysteines needed to disulfide bond together and form a loop structure. This design is the same as the sequence used to make synthetic peptide mimic Man₉ V3, except this design is glycosylated at positions N295, N301, and N332, whereas the previous synthetic peptide was only glycosylated at N301, N332 or both. In these designs, the peptide sequence is derived from clade B.JR-FL, but it can be varied to match envelope sequences from any HIV-1 envelope or an artificially designed combination of envelope amino acids. Sequences were codon optimized in order to maximize expression by the HEK293 cell line. The glycopeptide antigen is multimerized on a protein nanoparticle, in this case H. pylori ferritin. The glycopeptide has a short glycine-serine linker that can vary from 2 amino acids up to 5 amino acids appended to its C-terminus to allow it to flexibly attach to the ferritin chain. The glycopeptide and glycine-serine linker is attached to the Asp4 of the H. pylori ferritin chain to orient the glycopeptide away from the nanoparticle surface. Assembled nanoparticles were visualized by negative stain electron microscopy. Reference-free 2D class averages were generated from raw micrographs. Enlarged image shown in bottom right. Scale bar 200 nm. Images confirm successful assembly of ferritin nanoparticles displaying glycopeptides with glycopeptides arrayed on the surface (FIGS. 8-12).

The second minimal antigen designed to recapitulate the V3-glycan site is the same as disclosed herein except it is elongated to include the N339 glycosylation site. The glycopeptide is derived from HIV-1 envelope amino acids 293 through 304 (EINCTRPNNNTR) and 320 through 343 (GEIIGDIRQAHCNISRAKWNDTLK). These two stretches of amino acids are joined together by a proline, which facilitates a beta-turn of the glycopeptide structure. The resultant glycopeptide has glycosylation sites at positions 295, 301, 332, and 339, hence it is termed tetraglycoV3. The glycopeptide antigen is multimerized on a protein nanoparticle. The glycopeptide has a short glycine-serine linker that can vary from 2 amino acids up to 5 amino acids appended to its C-terminus to allow it to flexibly attach to a protein nanoparticle. For H. pylori ferritin, the glycopeptide and glycine-serine linker is attached to the Asp4 of the H. pylori ferritin chain to orient the glycopeptide is on the outside of the nanoparticle.

The third glycopeptide minimal immunogen encompasses the portion of Env that has glycosylation sites at positions 386 and 392. These glycosylation sites are located within the fourth variable (V4) region of HIV-1 envelope. Thus, the designed immunogen is termed glycoV4. We designed three different versions of the glycoV4 antigen. Each design differs in how the flexible tip of the V4 loop is truncated. In one design the full-length V4 loop is included. This construct encompasses amino acids 382 to 421 (FFYCNSTQLFNSTWNNNTEGSNNTEGNTITLPCRIK). This construct includes glycosylation sites at 386 and 392, which are bound by V3-glycan broadly neutralizing antibody PGT135 and Fab-dimerized glycan broadly neutralizing antibody DH851.2. It also includes additional glycans and the tip of the V4 loop that is not required for broadly neutralizing antibody binding. Thus, we truncated the center of the V4 loop to eliminate immunogenic peptide sequence and two glycosylation sites not utilized by broadly neutralizing antibodies. These deletions created glycoV4min.1. We further truncated the N-terminus and C-terminus by 3 amino acids each to eliminate additional peptide sequence that can be unnecessary. This final construct was called glycoV4min.2.

In these glycoV4 designs, the peptide sequence is derived from clade B.JR-FL but it can be varied to match envelope sequences from any HIV-1 envelope. The glycopeptide antigen is multimerized on a protein nanoparticle, in this case H. pylori ferritin. The glycopeptide has a short glycine-serine linker that can vary from 2 amino acids up to 5 amino acids appended to its C-terminus to allow it to flexibly attach to the ferritin chain. The glycopeptide and glycine-serine linker is attached to the Asp4 of the H. pylori ferritin chain to ensure the glycopeptide is on the outside of the nanoparticle.

Glycoengineering. The glycans on the minimal antigens can be engineered to interact optimally with antibodies. This glycoengineering can be achieved by producing the glycopeptide nanoparticles in cell lines lacking specific glycosidases like GnT1; the addition of glycosylation inhibitors such as kifunensine or swainsonine to the culture medium; the addition of glycosidases to purified proteins; or by selective antibody affinity purification of the glycoform during protein purification. These methods can be used to most accurately mimic the glycan composition found on native HIV-1 envelope.

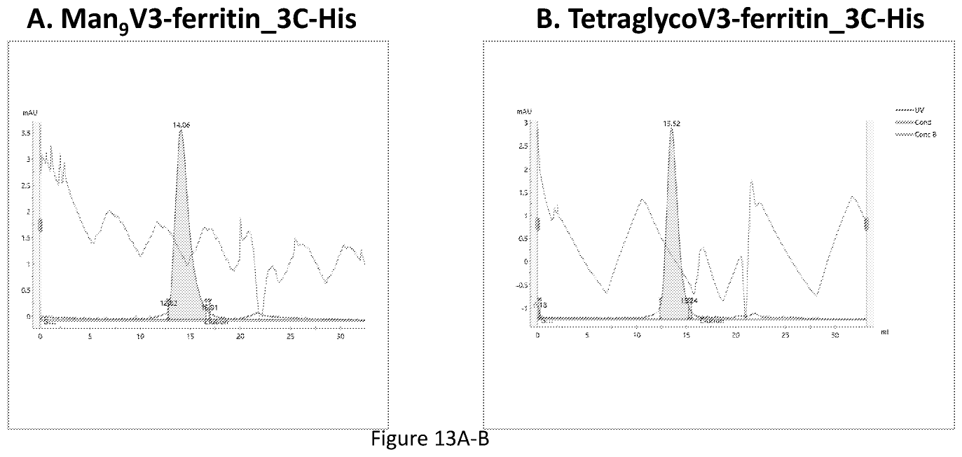

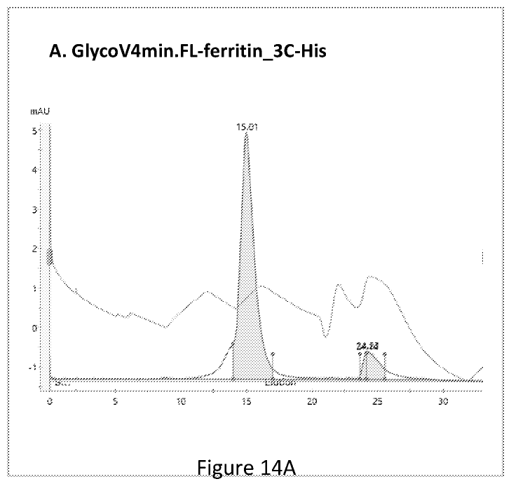

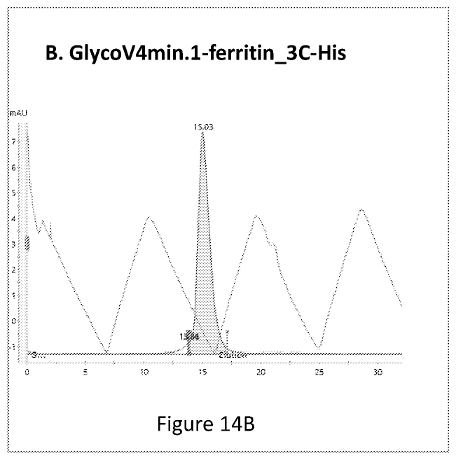

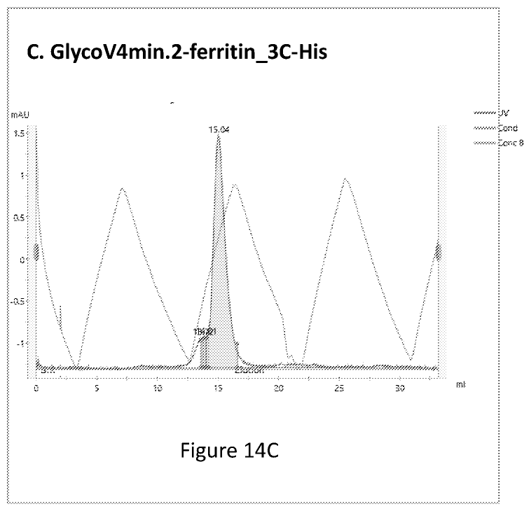

Glycopeptide nanoparticle production (FIGS. 13-16). The glycopeptide nanoparticles are expressed as recombinant proteins in mammalian cells so that mammalian glycans are added to the peptide. These cells include but are not limited to 293 and their derivatives such as Freestyle 293F cells. In some cases, the glycans on the glycopeptide were restricted to MangGl₉NAc₂ or Man₈GlcNAc₂ by kifunensine treatment. Mass spectrometry showed the glycopeptide had mostly Man₉GlcNAc₂ on it. Nanoparticles are secreted from the cells during protein expression. The cell culture supernatant was harvested and cleared of cells. Cell-free culture supernatant was purified by affinity chromatography using glycopeptide specific antibodies or by a tag. Size exclusion chromatography was used to separate glycopeptide nanoparticles from other protein types. Analytical size exclusion of collected protein showed the purified protein was a single peak of homogenous protein consistent with the size of a nanoparticle. (FIGS. 13-14).

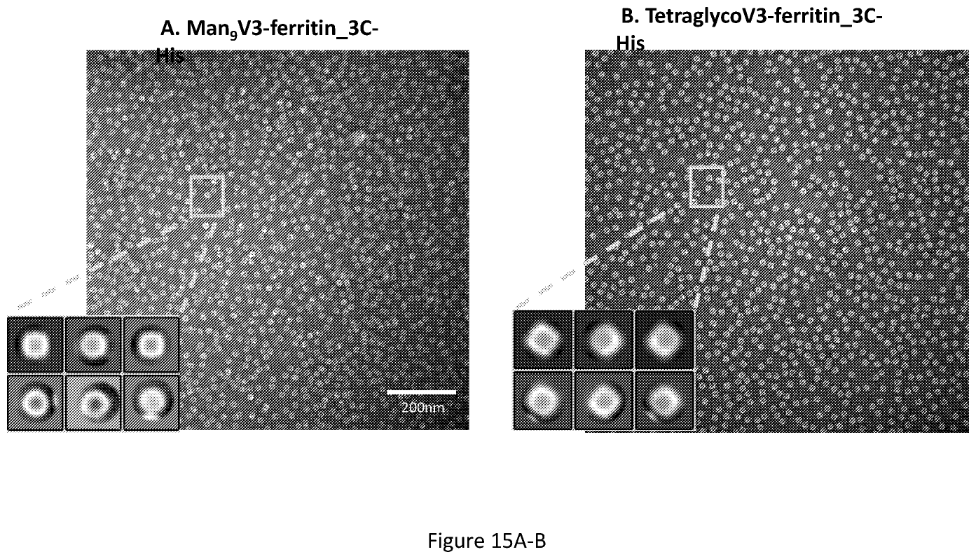

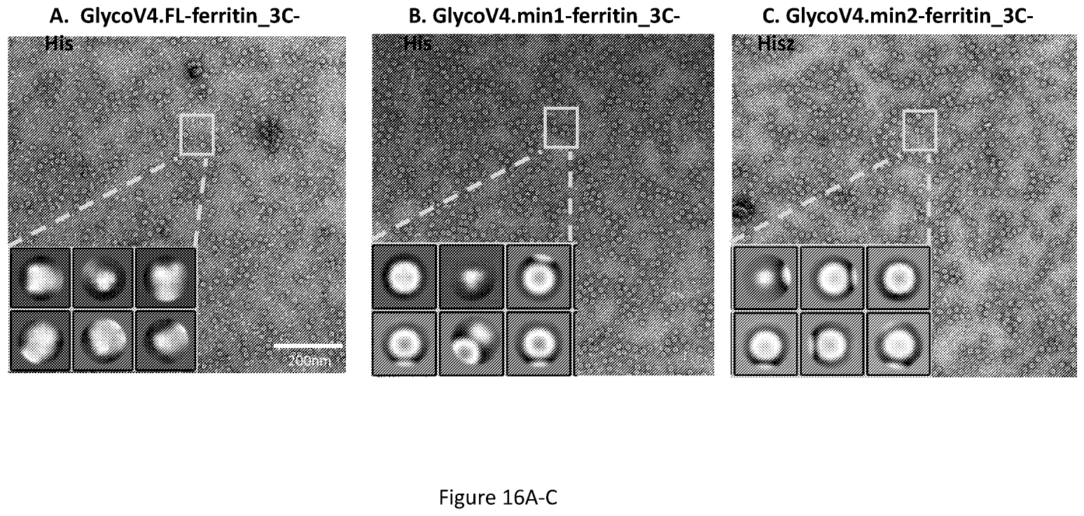

Visualization of assembled glycopeptide nanoparticles: To ensure incorporation of glycopeptide sequences did not disrupt nanoparticle formation we used negative stain electron microscopy to examine morphology. Purified nanoparticle samples were applied to a mesh carbon electron microscopy grid and stained with 0.5% uranyl fomate. Enlarged images show 2D class averages generated from raw micrographs. (FIGS. 15-16) Each construct design yielded homogenous assembled particles.

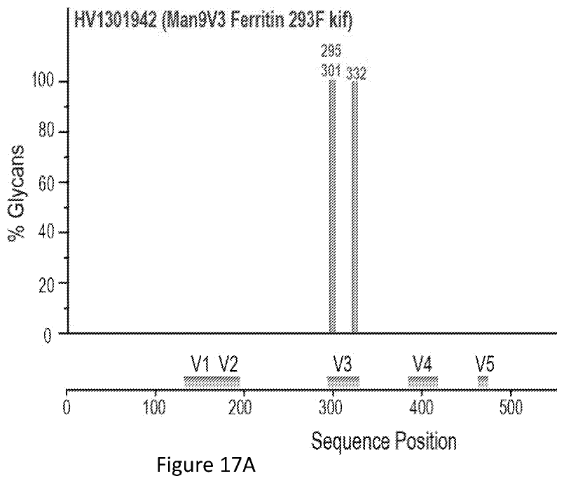

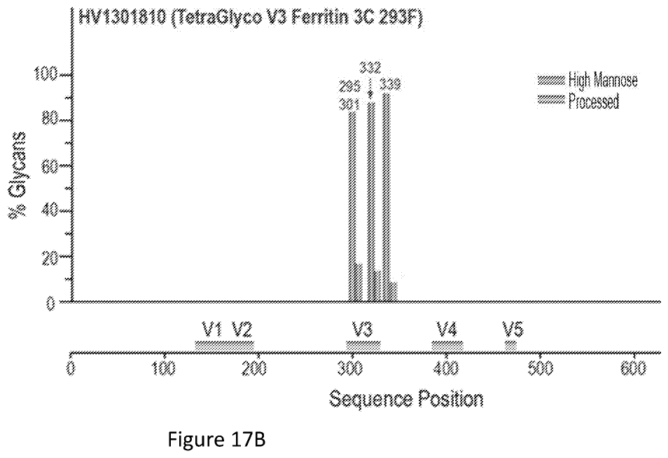

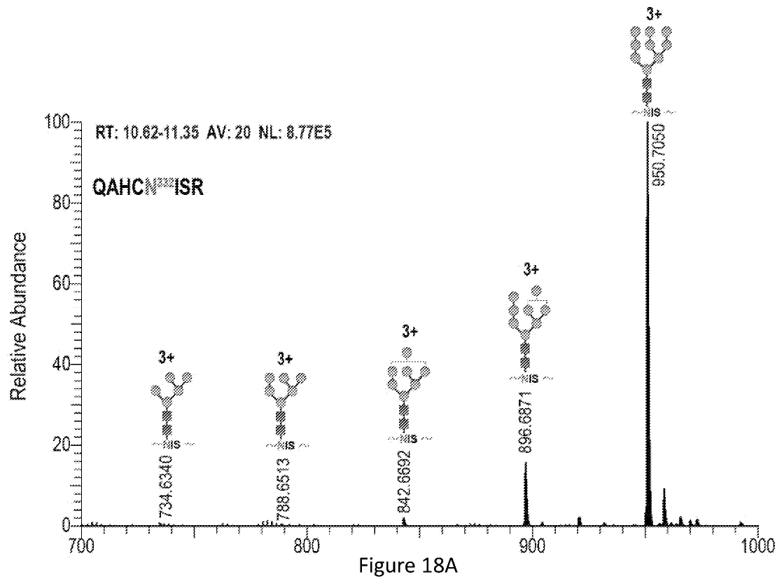

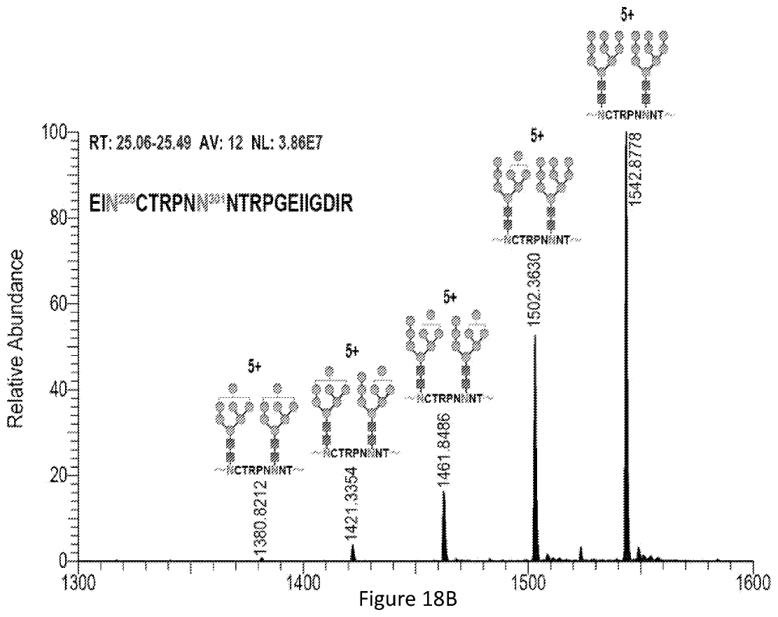

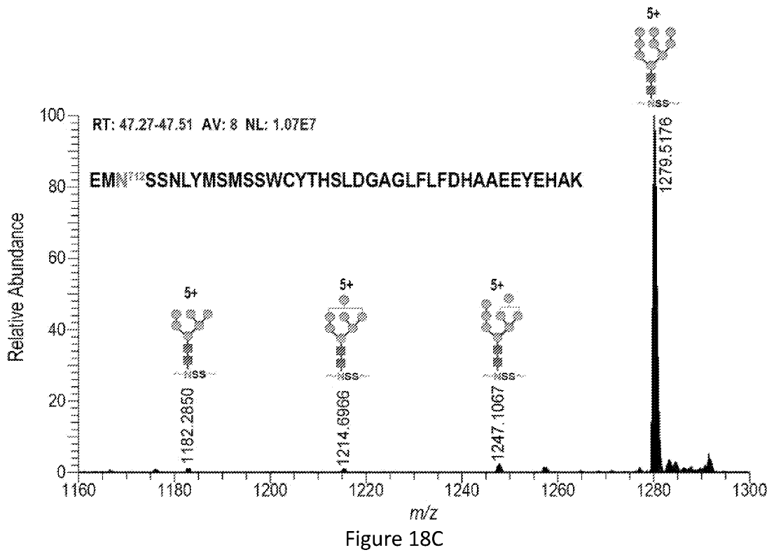

Glycoprofiling of glycopeptide nanoparticles (FIGS. 17-18): Mass spectrometry was used to interrogate glycan occupancy at potential N-linked glycosylation sites and to determine percent composition of glycan species at each sequence position for Man9V3-ferritin and TertraGlycoV3. Relative high mannose (red) and processed glycan (blue) composition represented as bars. The Man9V3-ferritin nanoparticles were produced in cells that were treated with the glycosidase inhibitor, kifunensine. This treatment restricts enzymatic glycan trimming resulting in enrichment of high mannose glycoforms. Glycoprofiling data show Man9V3 displays exclusive expression of high mannose in clusters at the N295, N301 and N332 positions. In contrast, the V3 site of TetraglycoV3, which did not receive kifunensine pretreatment, is predominantly occupied by processed glycans interspersed with small amounts of high mannose. FIG. 18 shows relative amounts of various high mannose glycan present at each position. Both constructs show formation of a glycan cluster surrounding position N332. Relative abundance of high mannose glycoforms Mapping of N glycosylation on the representative glycopeptide nanoparticles is in accordance with the patterns reported for viral JR-FL Env (Cao, Nature Communication 2018). These figures show that the glycopeptide display similar glycosylation profiles as native HIV-1 Env

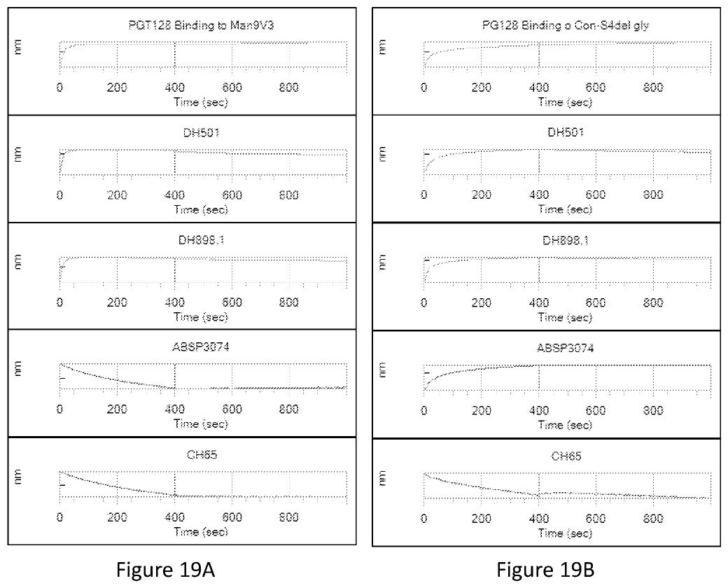

FIGS. 19-40 show that glycopeptide nanoparticles are antigenic for and targeted by HIV neutralizing antibodies.

Evaluation of Glycopeptide nanoparticle antigenicity: To ensure antigenic properties of glycopeptide nanoparticles were retained we evaluated binding to a panel of antibodies composed of potently neutralizing and non-neutralizing. Glycopeptides showed strong reactivity with neutralizing antibodies that target the V3 glycan epitope and failed to react with antibodies with specificities for poorly conserved peptide regions. The discrepancies in binding by these classes of antibodies verify that our design strategy is sufficient for removal of poorly antigenic regions while retaining required regions 1 for neutralization. These results were obtained using multiple binding assay platforms and experimental set ups including direct and indirect ELISAs, biolayer interferometry and absorbance assays.

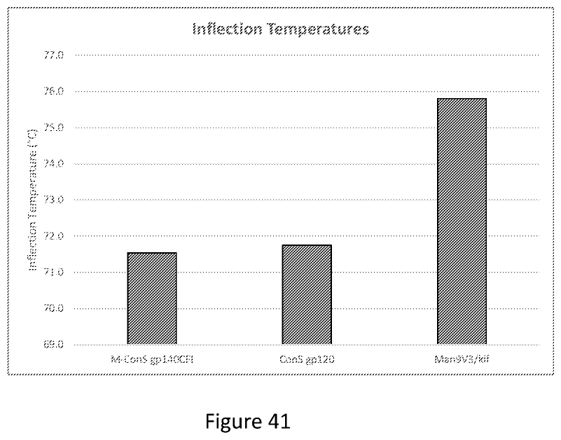

Thermostability of Man₉V3-ferritin glycopeptide nanoparticles (FIG. 41): Native Env is inherently unstable, undergoing dynamic transitions between different conformational states. These structural changes introduce a temporal component to accessing key epitopes and make viral neutralization by antibodies more challenging, as epitopes cannot always be exposed. Consequently, stable structures are more favorable. Nanoparticle scaffolds represent one method to promote delivery and stabilization of immunogens in near native conformations. We used differential scanning fluorescence to assess the stability of our nanoparticles as measured by thermal unfolding temperature. As proteins unfold, tryptophans become exposed that fluoresce. Samples were subjected to increasing temperatures to determine the temperature required to destabilize intramolecular bonds. Differences in fluorescence intensity after incubation reveal the degree of unfolding by each construct tested. Man9V3-ferritin demonstrated the highest melting temperature when compared to monomeric CON-S Env and trimeric CON-S. Man9V3 was able to withstand higher temperatures before unfolding indicating it is stable.

Potential uses as a priming, boosting, or sequential vaccine. The glycopeptide immunogens can be used in prime-boost regimens to boost glycan-dependent antibody responses. One example of such use is to boost with glycopeptide nanoparticles after HIV-1 envelope trimer immunization. We performed this regimen in rabbits and found that after one boosting immunization, all rabbits generated glycan-dependent binding to Man9V3 glycopeptide. The glycopeptide nanoparticle can also be given as a prime immunogen to only elicit HIV-1 antibodies to one site. Subsequently, those antibodies can be boosted with a HIV-1 envelope trimer to select trimer-reactive antibodies.

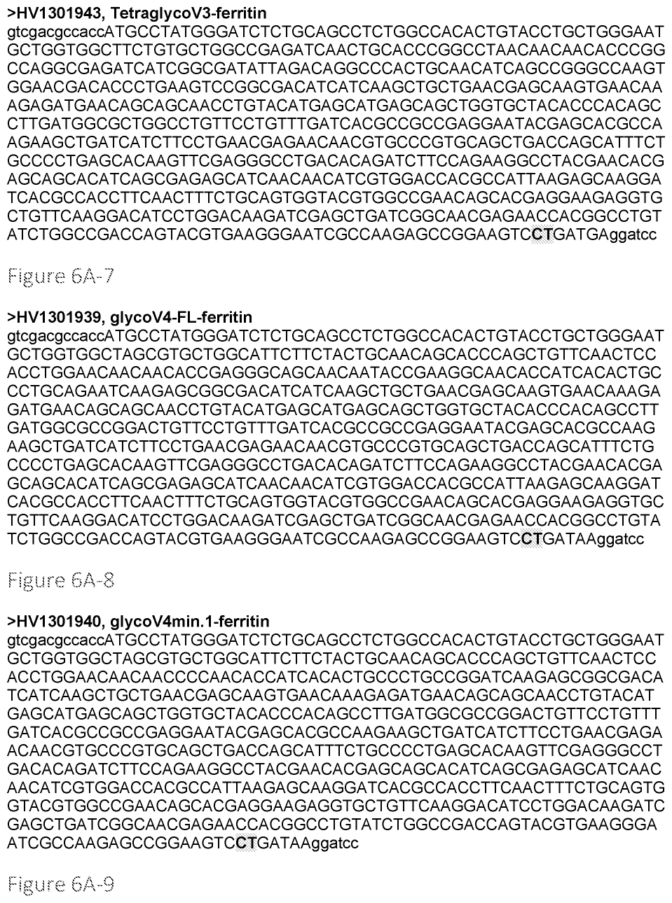

In a third approach, the different glycopeptide nanoparticles are administered in sequence to guide affinity maturation of glycan-reactive antibodies (FIG. 5). We have found that antibodies such as DH270 and PGT128 bind to high mannose glycan early in their affinity maturation process, and later develop the ability to recognize heterogenous glycans. Thus, a sequential vaccine can include immunizing with Man₉V3 glycopeptide nanoparticle to prime glycan-dependent antibodies and then boosting with tetraglycoV3 in order to broaden the number of glycans recognized and finally boosting with different glycoforms of TetraglycoV3 in order to broaden the types of glycans antibodies can recognize. This strategy is amenable to induction of FDG antibodies (FIG. 5).

TABLE 2
Uses of glycopeptide nanoparticles to elicit
Fab dimerized glycan neutralizing antibodies.

| Immunogen | Epitope | Glycans Present | Purpose |
|---|---|---|---|
| Man9V3 | V3 loop | N332, N301, N295 | Present Man9 glycoforms |
| | | | of V3 glycans |
| glycoV3 | V3 loop | N332, N301, N295 | Present heterogenous |
| | | | glycoforms of V3 |
| | | | glycans |
| TetraglycoV3 | V3 loop | N332, N301, N295 | Present an increased |
| | | N339 | number of heterogenous |
| | | | V3 glycans |
| GlycoV4 | V4 loop | N386, N392 | Present V4 glycans |
| (FL, min.1, | | | recognized by FDG |
| min.2) | | | bnAbs |

Immunogenicity of glycopeptide nanoparticles: We performed a vaccination study using an Env prime, nanoparticle boost strategy to assess the immunogenicity of our lead glycopeptide nanoparticle. 10 New Zealand white rabbits were immunized with 1 of two stabilize CON-S SOSIPs. Animals received 3×prime with SOSIP at 4 week intervals followed by 2×boost with Man9V3-ferritin_3C-His nanoparticle. ELISA assays were performed to detection of specific immune responses. Schematic of immunization schedule shown. Group 2 rabbits elicited high titers of autologous antibodies against priming immunogen after 3 doses. Boosting with glycopeptide nanoparticles maintained titers for 12 weeks after final SOSIP boost. 10/10 rabbits generated responses that can discriminate between glycosylated peptide constructs and identical constructs lacking glycans. These results provide or indicate glycan specific and dependent antibodies were elicited. When serum from immunized rabbits was assayed against the respective nanoparticle immunogen higher binding titers were obtained that against monomeric peptide. This data provides or indicates multimerization of immunogens is sufficient to enhance immune recognition. Antibodies elicited by nanoparticle boosts retained the ability to associate with epitopes as displayed on native-like Env SOSIIP. This result further confirms nanoparticle display of the V3 epitope to be an accurate mimic of the natural epitope.

Env Prime elicits antibodies that recognize glycopeptides: Binding data from mouse sera from two separate studies using different strategies. The former, mice were primed with repetitive doses of Env SOSIP. This strategy produced a dose dependent anti SOSIP response. Serum was tested against the series of designed glycopeptide nanoparticles. The constructs are able to engage preexisting antibodies elicited by whole Env immunization. Enrichment of high mannose was not required for recognition of the V3 and V4 constructs. This result provides or indicates repetitive Env prime is sufficient to select antibody lineages that acquire the ability to bind heterogeneous glycofoms and additional glycosylation sites. Further, subsequent boosting using minimal immunogens with progressively complex glycans can be necessary to select antibodies with the ability to acquire neutralization breadth. The highest responses targeted constructs containing heterogeneous glycoforms.

These data provide support for use of glycopeptides as boosting immunogens to drive affinity maturation of Env elicited antibodies towards broad Env recognition and neutralization potency. No reactivity was detected in serum from the adjuvant only group. Antibodies isolated from infected individuals were also able to engage the various glycopeptide constructs indicating the presence of precursor antibodies that can be targeted and affinity matured by vaccination.

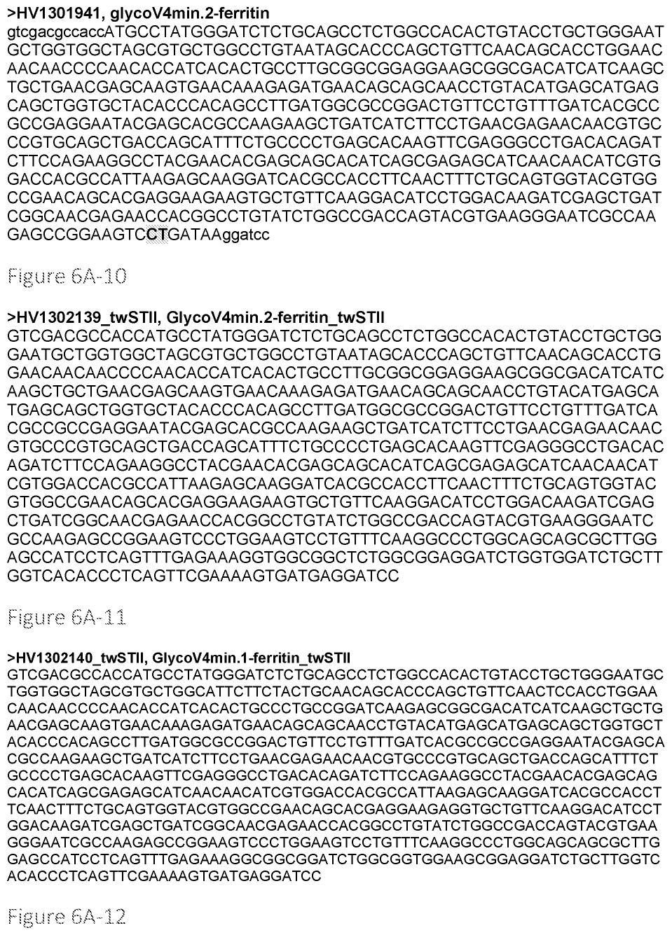

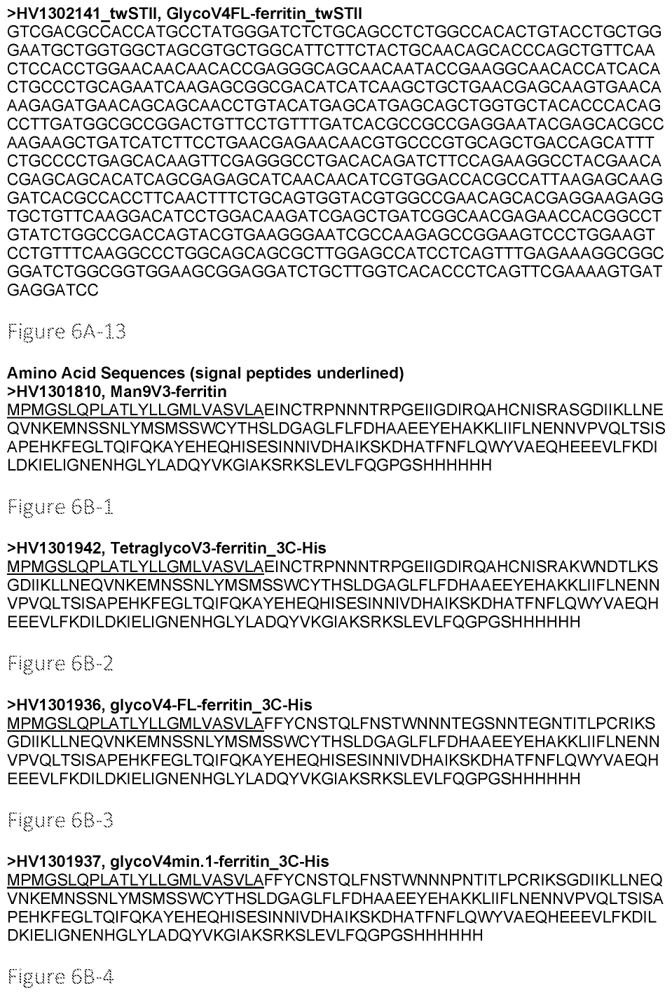

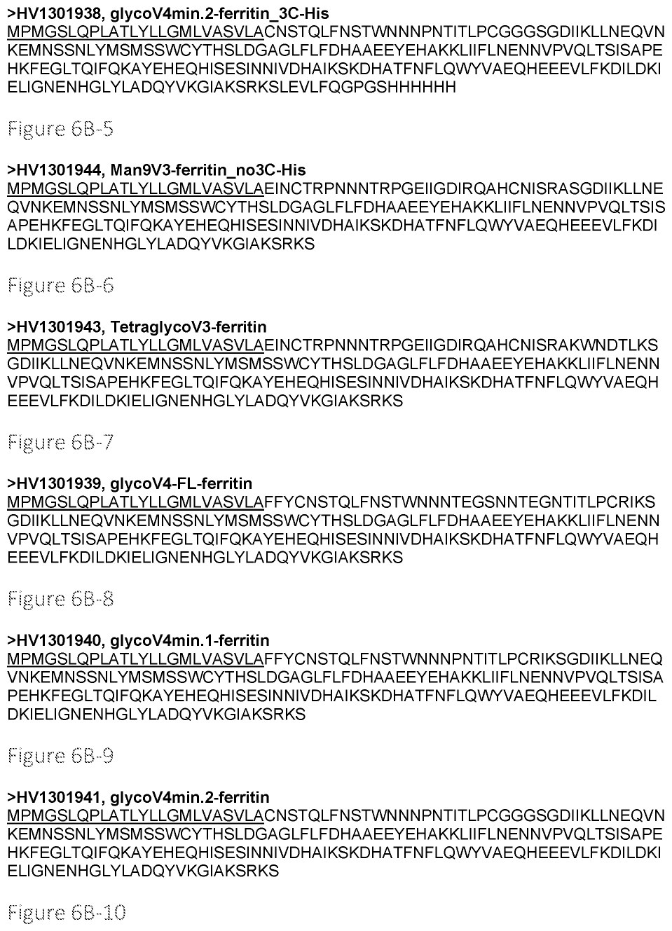

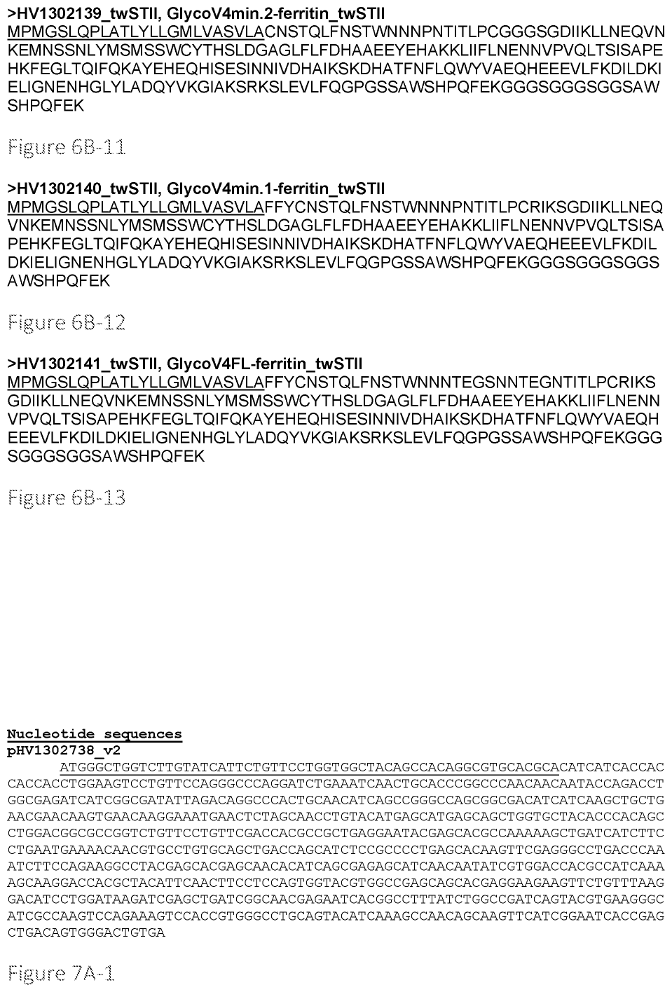

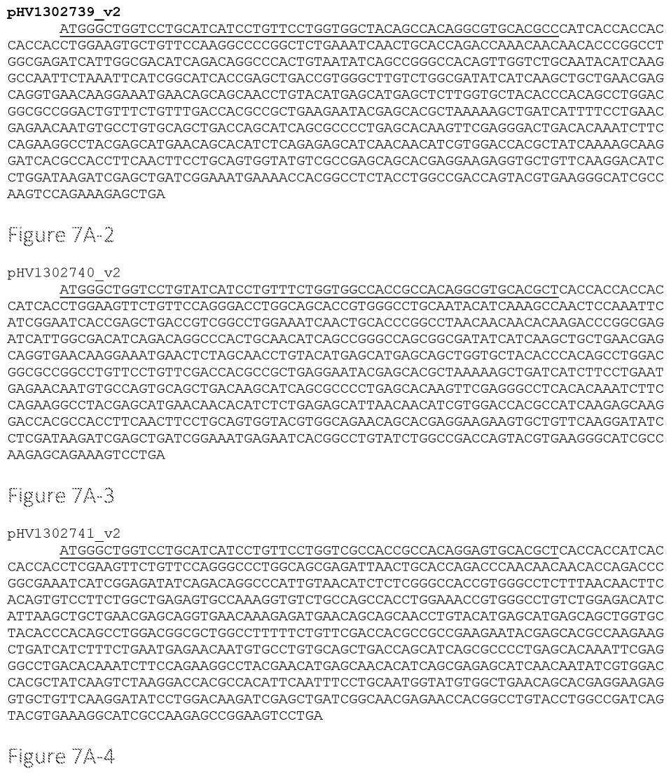

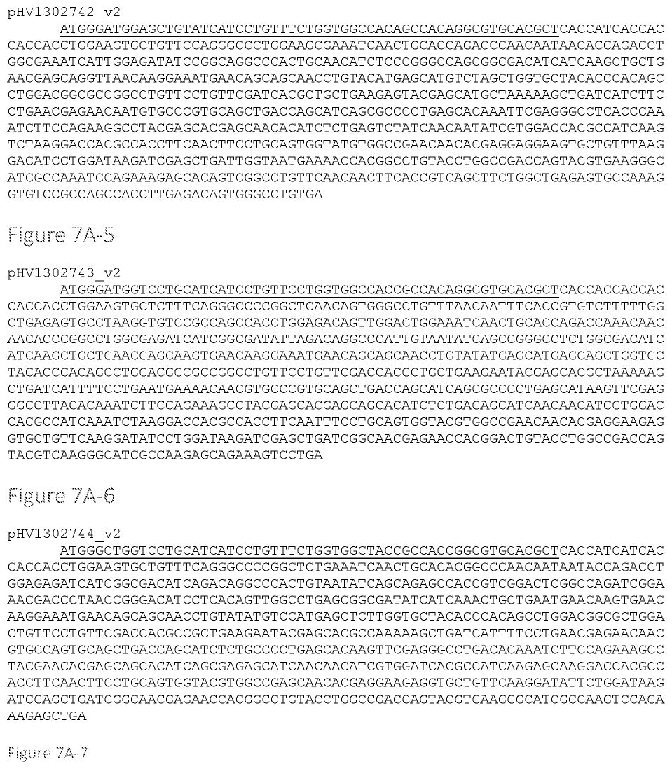

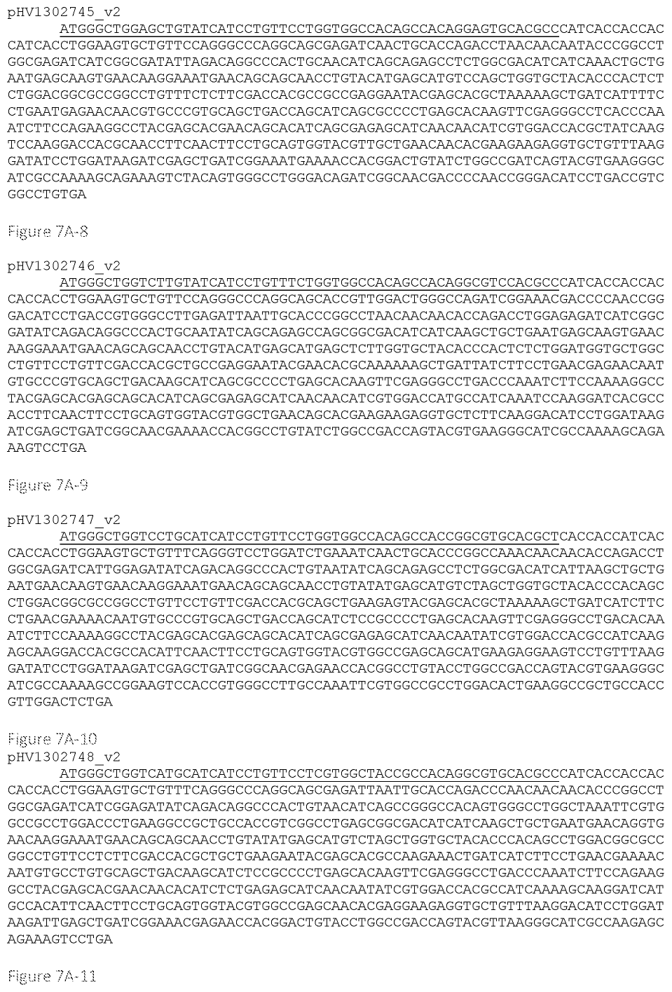

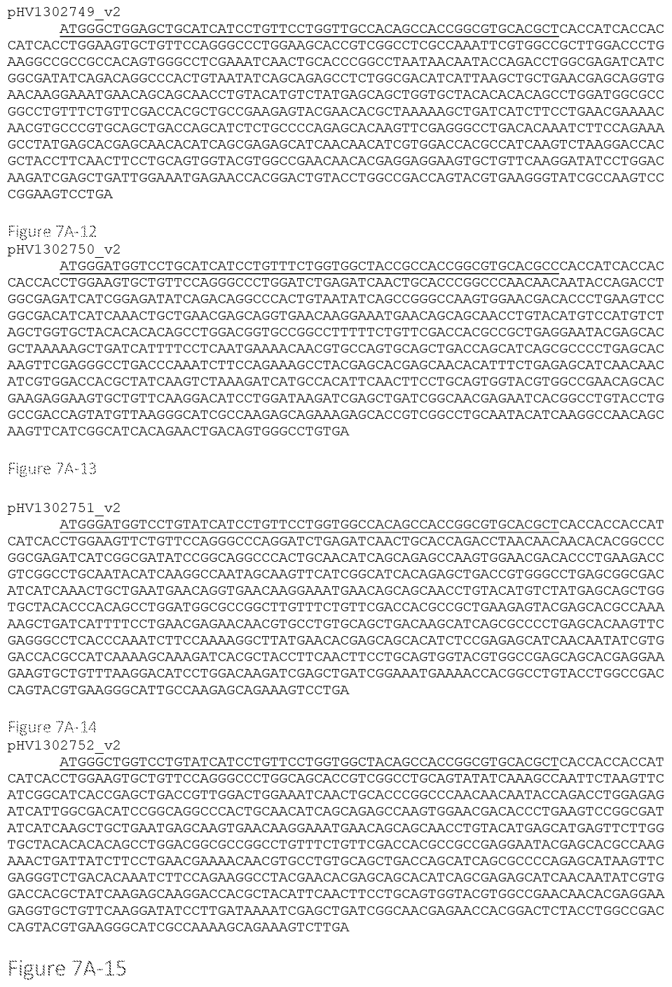

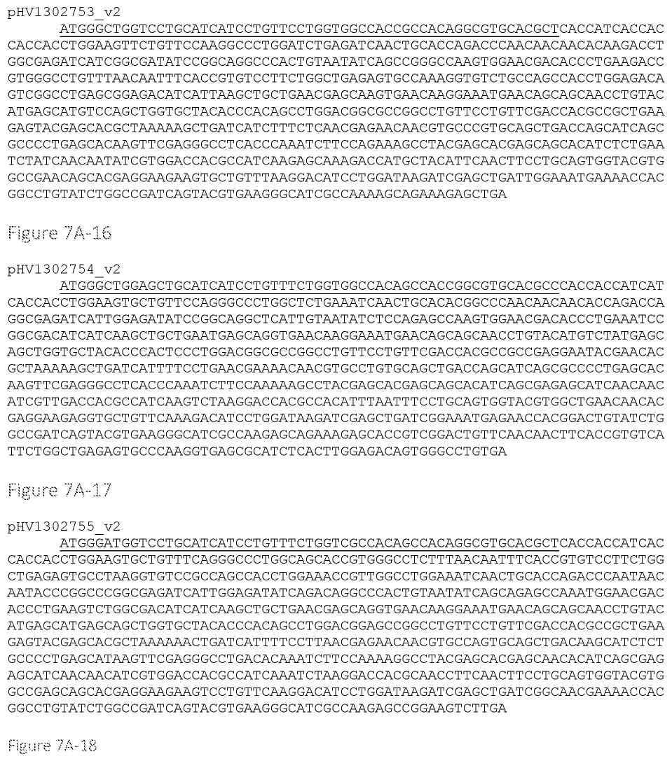

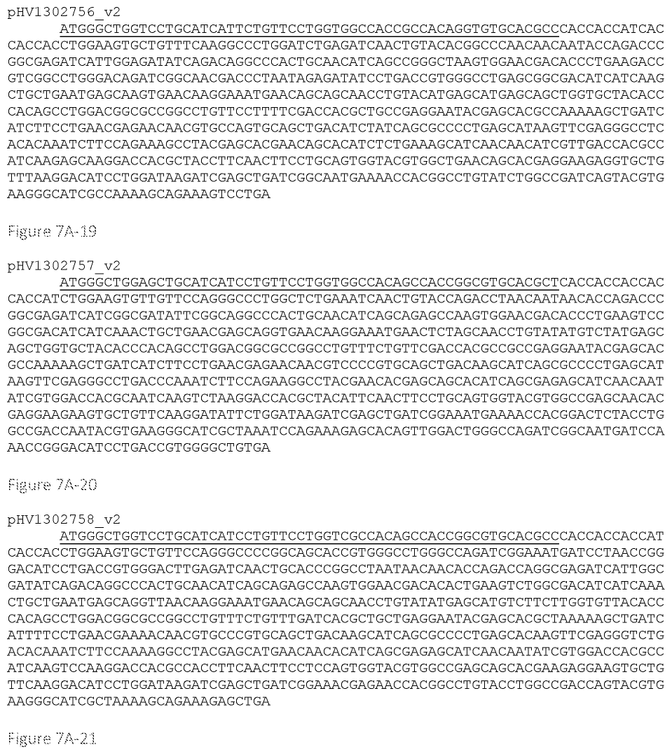

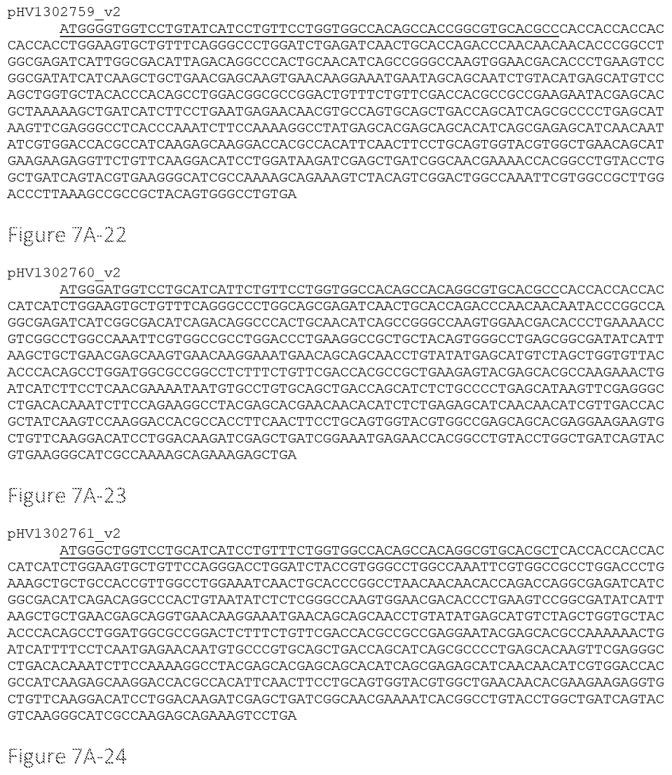

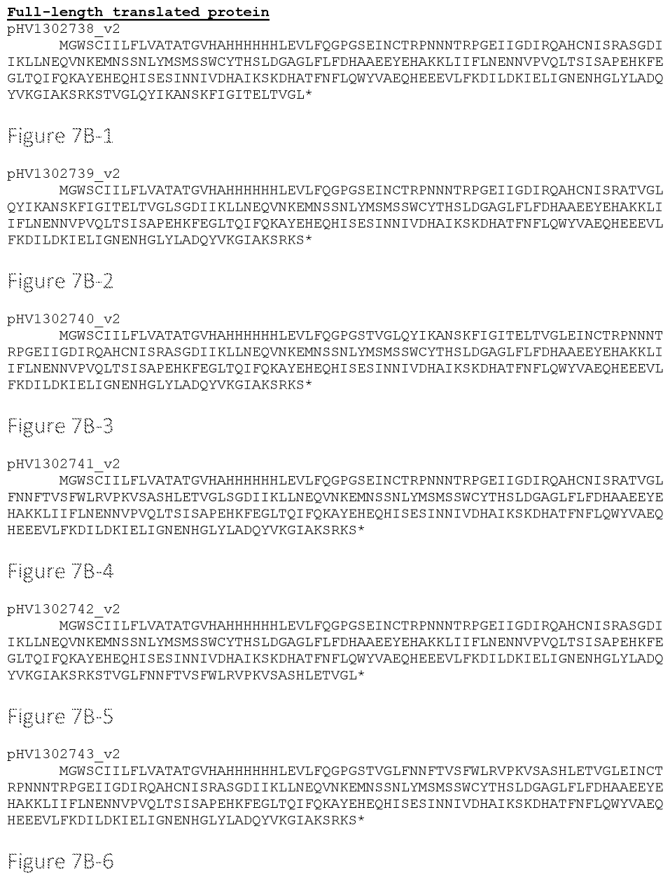

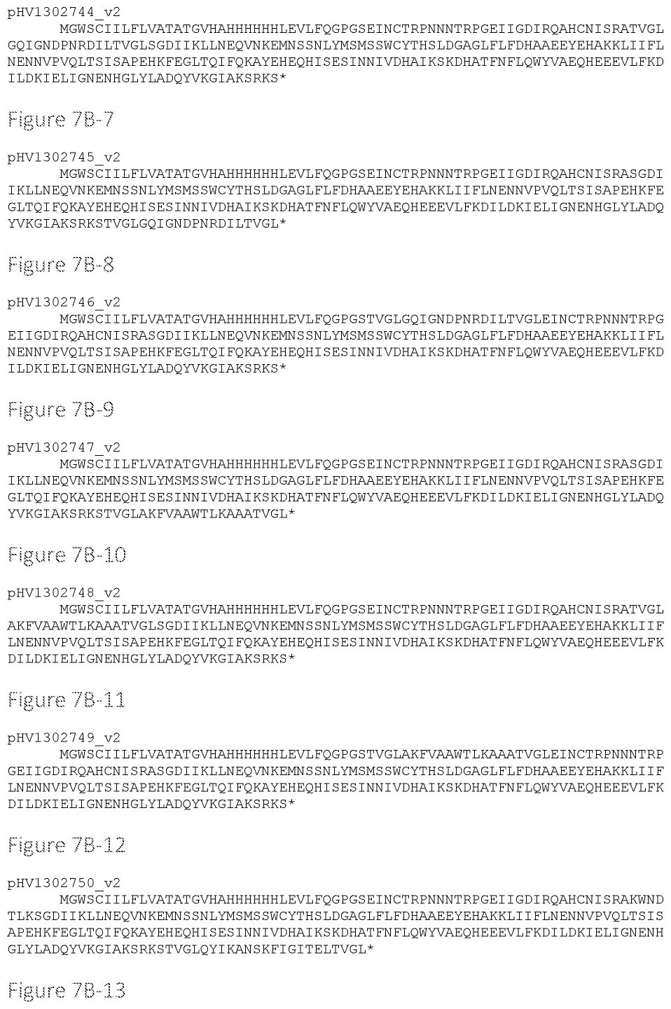

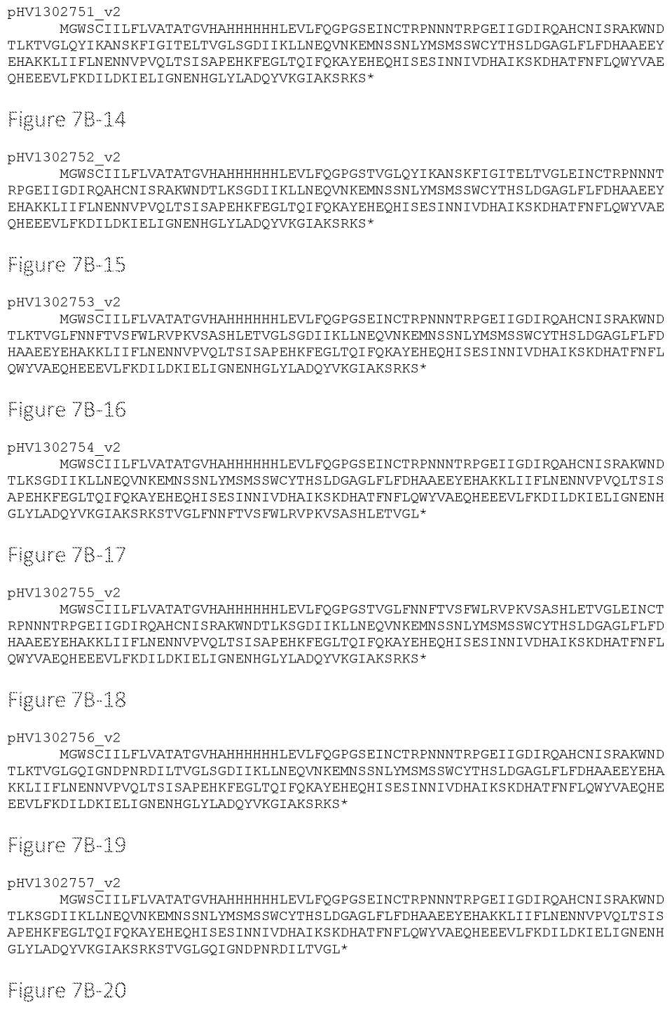

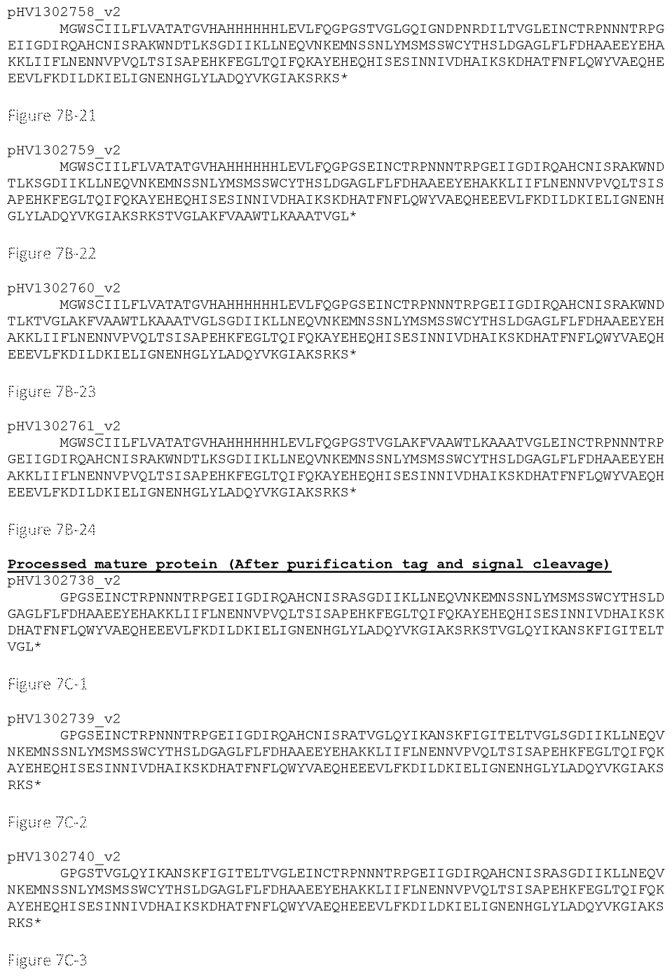

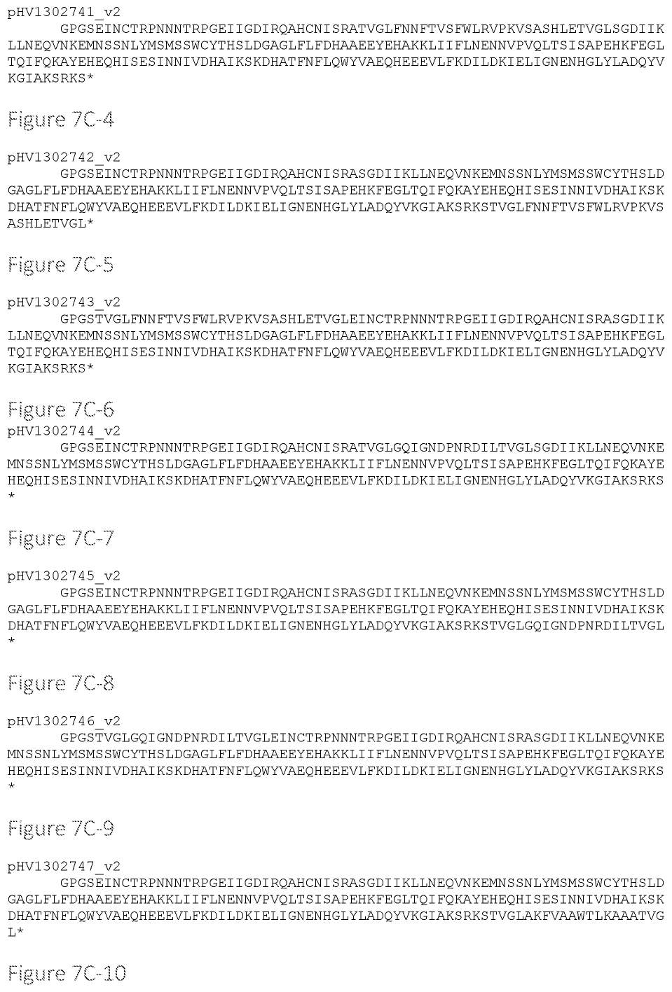

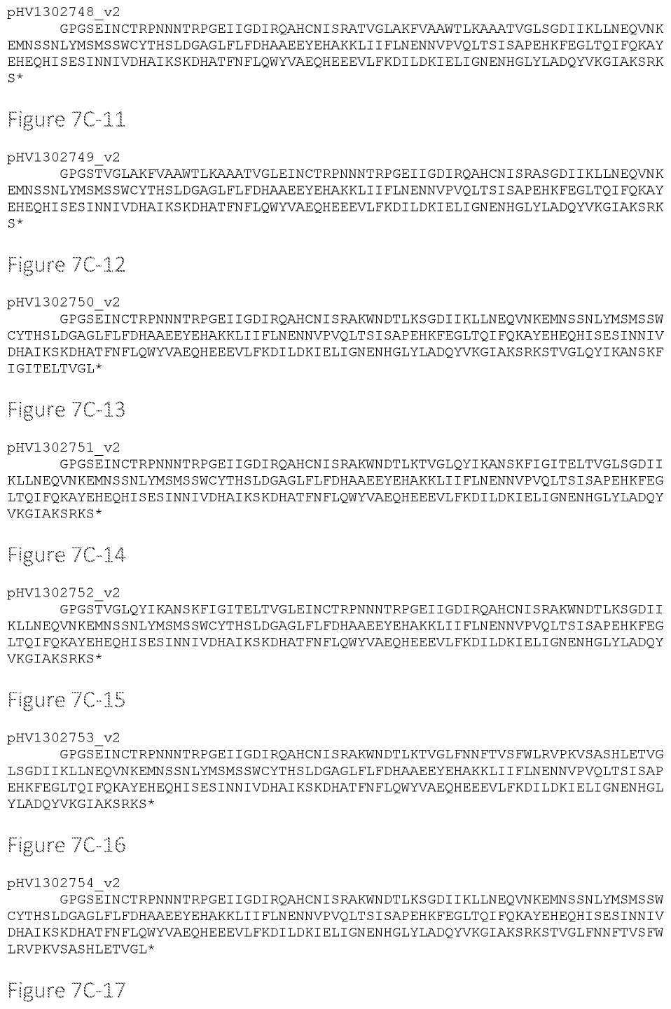

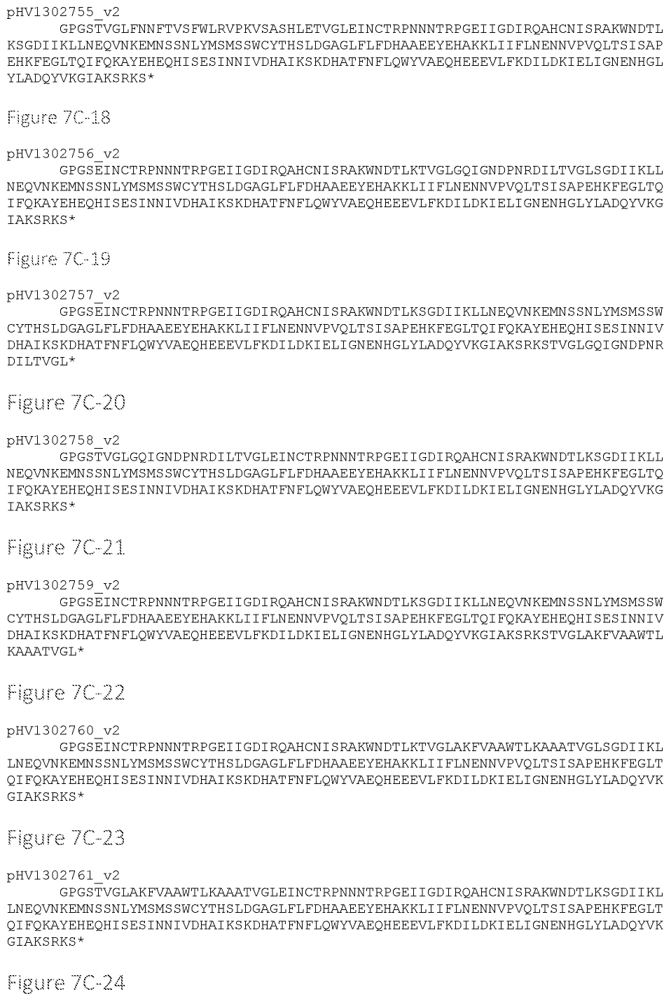

TABLE 3
Glycopeptide nanoparticle reference sequences. Non-limiting
embodiments of sequences are shown in FIGS. 6A-6B.

| Plasmid ID | Protein ID |
|---|---|
| HV1301810 | Man9V3-ferritin (this one is 3C-His) |
| HV1301942 | TetraglycoV3-ferritin_3C-His |
| HV1301936 | glycoV4-FL-ferritin_3C-His |
| HV1301937 | glycoV4min.1-ferritin_3C-His |
| HV1301938 | glycoV4min.2-ferritin_3C-His |
| HV1301944 | Man9V3-ferritin_no3C-His |
| HV1301943 | TetraglycoV3-ferritin |
| HV1301939 | glycoV4-FL-ferritin |
| HV1301940 | glycoV4min. 1-ferritin |
| HV1301941 | glycoV4min.2-ferritin |
| HV1302139 | GlycoV4min.2-ferritin_twSTII |
| HV1302140 | GlycoV4min.1-ferritin_twSTII |
| HV1302141 | GlycoV4FL-ferritin_twSTII |

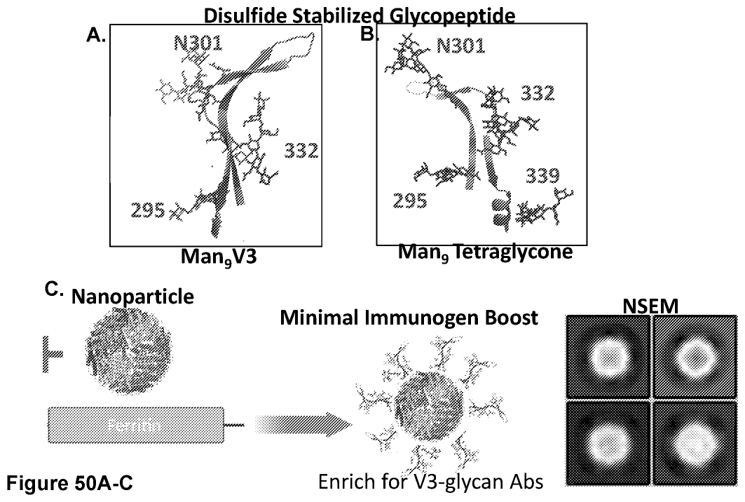

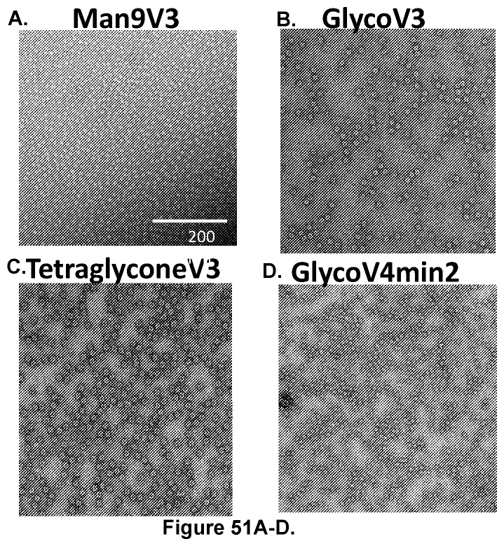

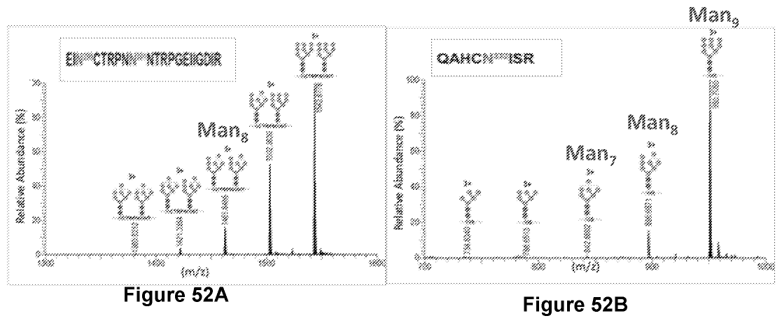

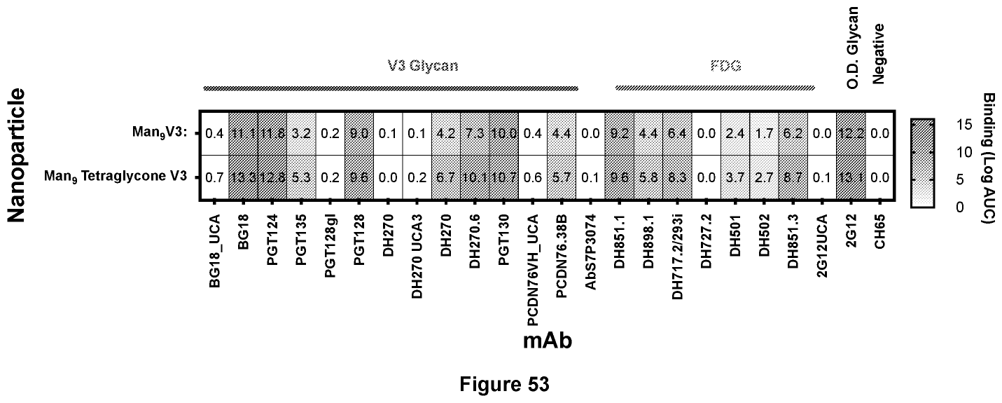

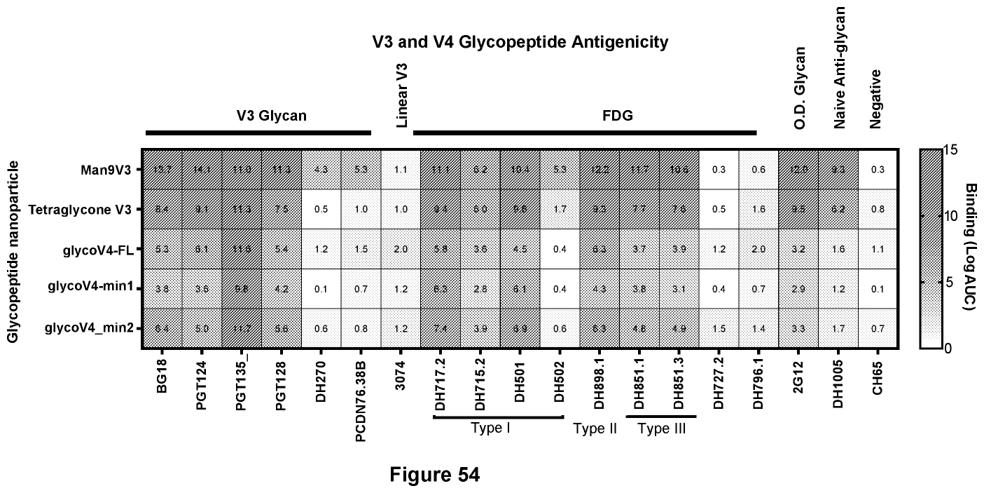

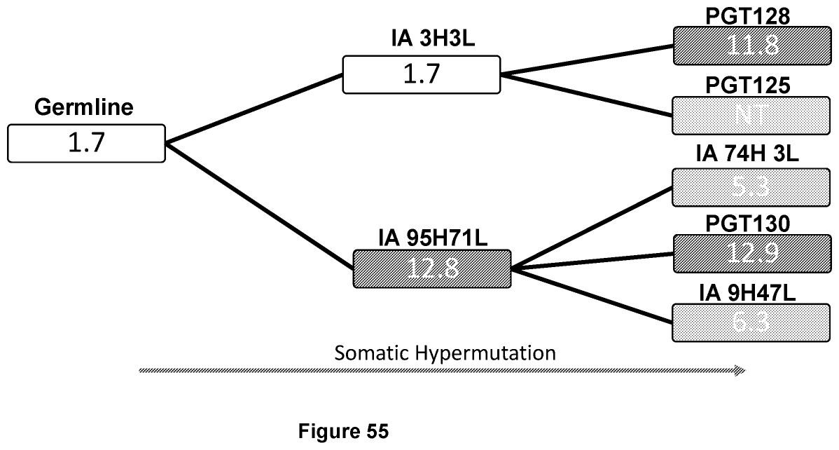

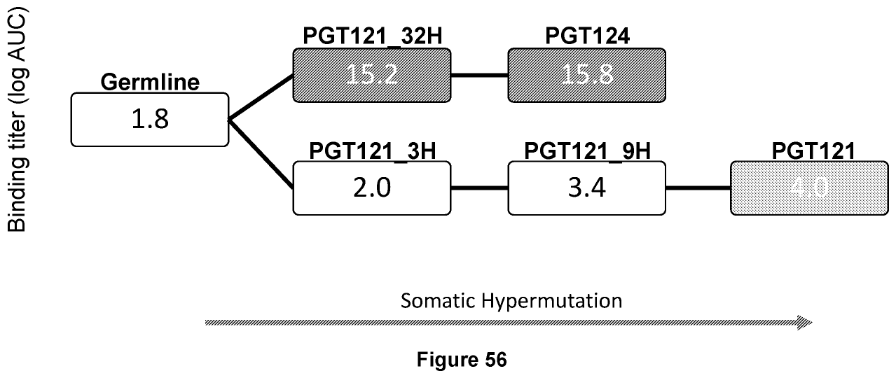

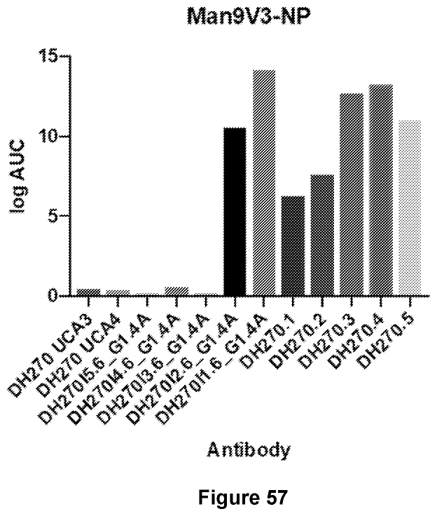

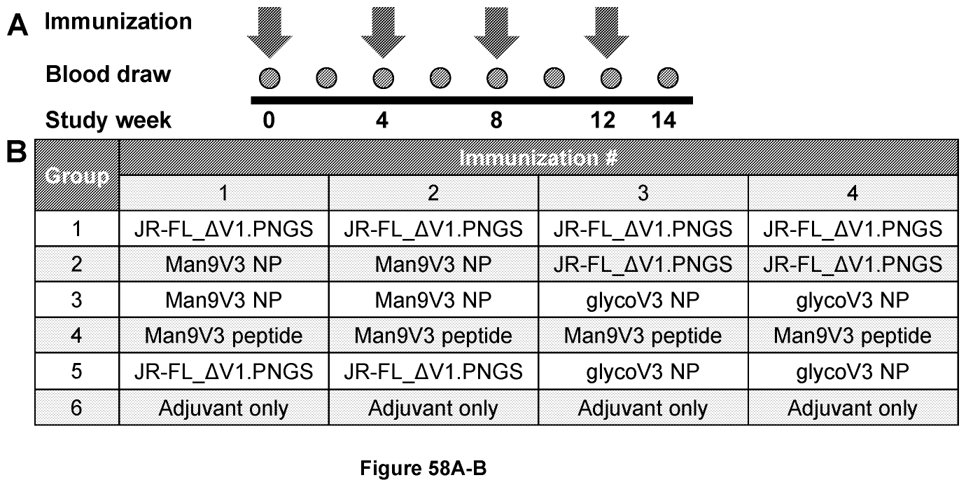

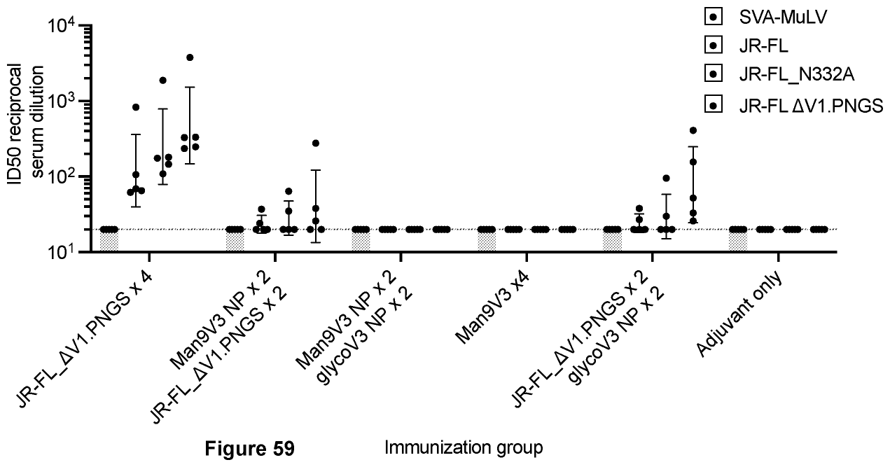

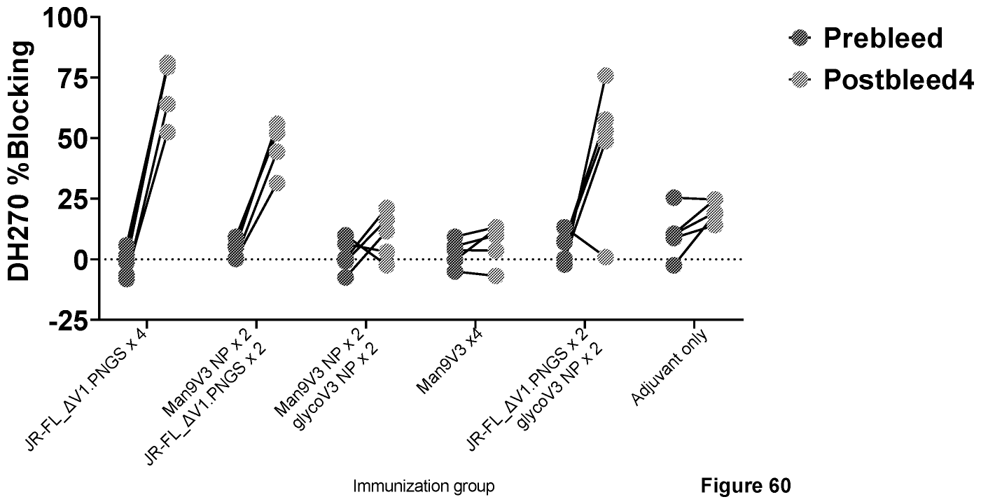

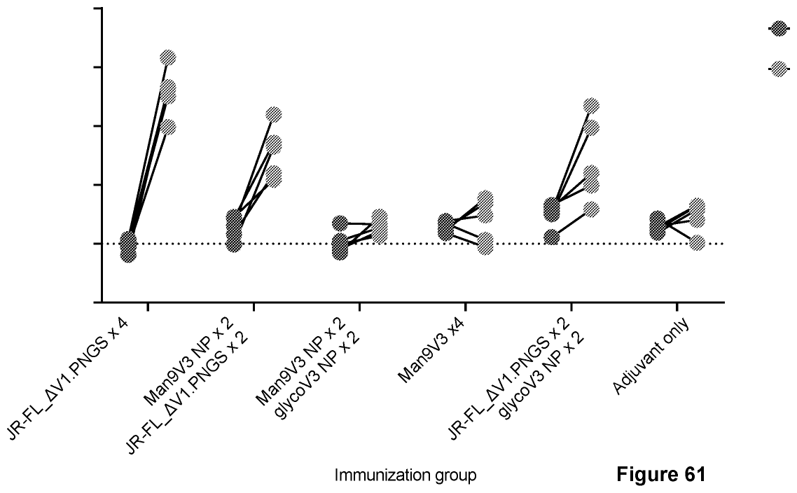

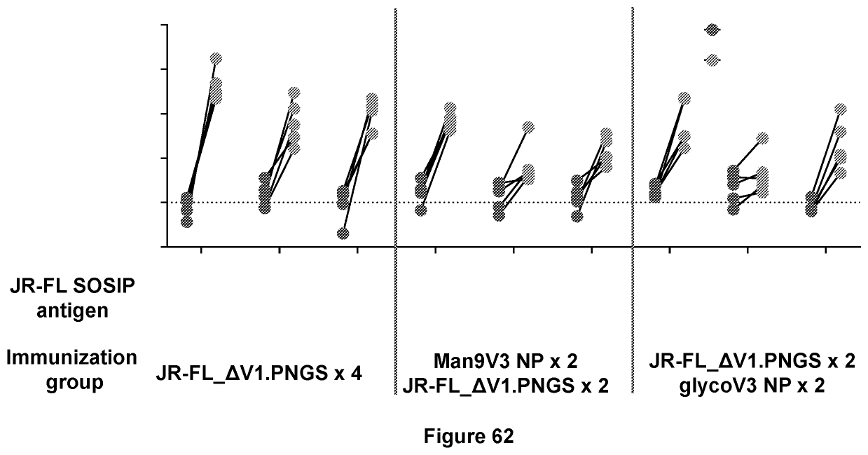

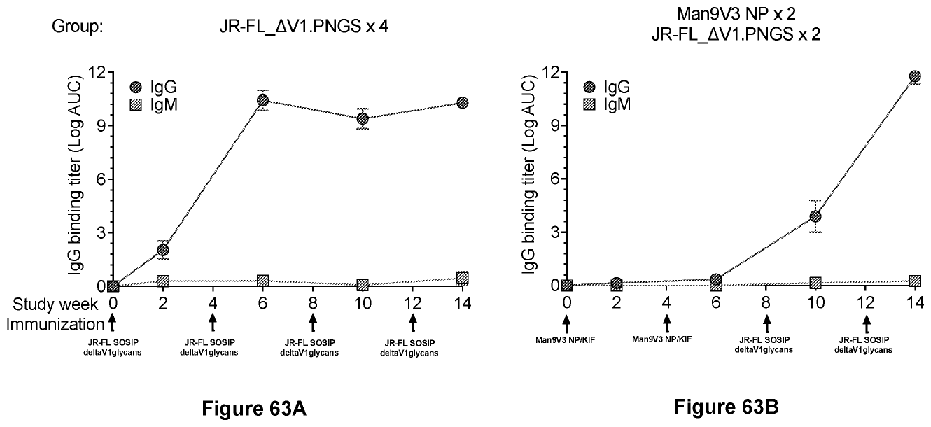

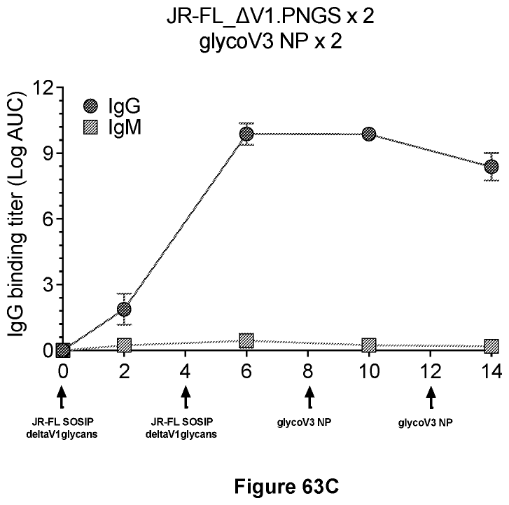

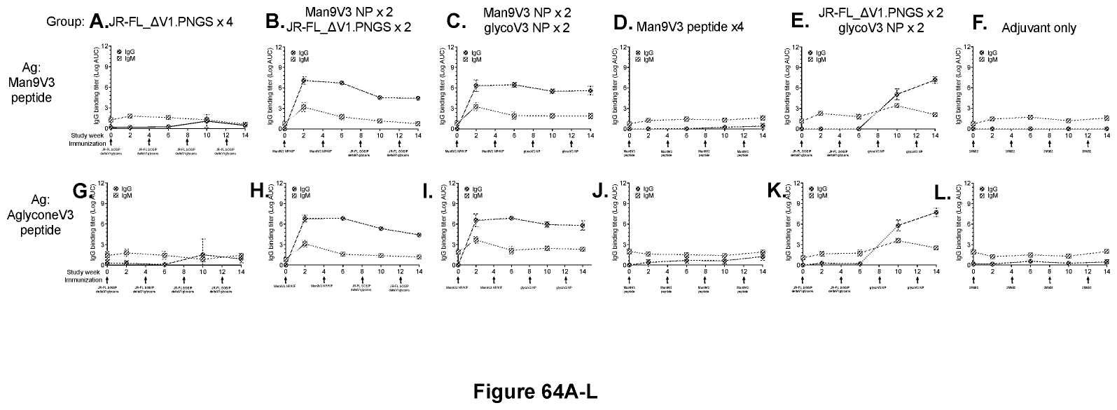

FIG. 50-64 show designs and analyses of glycopeptide nanoparticles.

### Example 2: Glycopeptide Nanoparticle Sequences with T-Cell Epitopes

TABLE 4
Glycopeptide nanoparticle sequences with T-cell epitopes.

| Protein ID | Protein Name |
|---|---|
| HV1302738_v2 | Man9V3-ferritin830TT_3C-His |
| HV1302739_v2 | Man9V3-830TT-ferritin_3C-His |
| HV1302740_v2 | 830TTMan9V3-ferritin_3C-His |
| HV1302741_v2 | Man9V3-947TTferritin_3C-His |
| HV1302742_v2 | Man9V3-ferritin947TT_3C-His |
| HV1302743_v2 | 947TTMan9V3-ferritin_3C-His |
| HV1302744_v2 | Man9V3-1273TTferritin_3C-His |
| HV1302745_v2 | Man9V3-ferritin1273TT_3C-His |
| HV1302746_v2 | 1273TTMan9V3-ferritin_3C-His |
| HV1302747_v2 | Man9V3-ferritinPADRE_3C-His |
| HV1302748_v2 | Man9V3-PADREferritin_3C-His |
| HV1302749_v2 | PADREMan9V3-ferritin_3C-His |
| HV1302750_v2 | TetraglycoV3-ferritin830TT_3C-His |
| HV1302751_v2 | TetraglycoV3-830TTferritin_3C-His |
| HV1302752_v2 | 830TTTetraglycoV3-ferritin_3C-His |
| HV1302753_v2 | TetraglycoV3-947TTferritin_3C-His |
| HV1302754_v2 | TetraglycoV3-ferritin_947TT3C-His |
| HV1302755_v2 | 947TTTetraglycoV3-ferritin_3C-His |
| HV1302756_v2 | TetraglycoV3-1273TTferritin_3C-His |
| HV1302757_v2 | TetraglycoV3-ferritin 1273TT_3C-His |
| HV1302758_v2 | 1273TTTetraglycoV3-ferritin_3C-His |
| HV1302759_v2 | TetraglycoV3-ferritinPADRE_3C-His |
| HV1302760_v2 | TetraglycoV3-PADREferritin_3C-His |
| HV1302761_v2 | PADRETetraglycoV3-ferritin_3C-His |

Minimal immunogens can focus immune responses to specific epitopes. However, minimizing the antigen size can result in no or few T cell helper epitopes being present. Thus, exogenous T cell helper peptides have been identified that can be added to minimal antigens to elicit T cell helper responses (PMID: 10640784). Peptides from tetanus toxoid that are presented to T cells by antigen presenting cells have been identified. These tetanus peptides may not be presented by all alleles of the major histocompatibility complex II (MHCII) molecules on the surface of antigen presenting cells. The PADRE 13 amino acid nonnatural peptide is an alternative peptide that can be presented by 15 of 16 MHCII molecules tested (PMID: 7895164 and PMID: 10640784). The addition of PADRE has improved immune responses for anti-glycan vaccines (PMID: 10640784 and PMID: 31206510).

The glycopeptide nanoparticle designs were constructed by adding a single T cell helper peptide to either of three positions. The first design added the T cell helper peptide at the N-terminus of the glycopeptide. The second design appended the T cell helper peptide at the C-terminus of the glycopeptide. The last design added the T cell helper peptide at the C-terminus of the ferritin nanoparticle subunit. The T cell helper peptides were flanked by a protease site (ThrValGlyLeu) which facilitate cleavage of the peptide for loading into MHCII molecules. Each nanoparticle was tagged at its N-terminus with a His purification tag (HHHHHH) since the C-terminus of the protein is inside the nanoparticle. The part of the molecule that targets HIV-1 antibodies is a peptide that is glycosylated at sites corresponding to N295, N301, N332, N339 on the HIV-1 envelope. Altogether the protein construct is a multimeric nanoparticle displaying both the glycans and peptides representing the V3 glycan site on HIV-1 envelope and an exogenous T cell helper peptide.

TABLE 5
Non-limiting embodiments of T cell
helper peptides added to the
glycopeptide nanoparticles.
Any other suitable helper
epitope can be used.

| | T | | |
|---|---|---|---|
| Peptide | epitope | Se- | |
| Name | Position | quences | Reference |
| 830TT | Tetanus | QYIKAN | Winberg, Cancer |
| | Toxoid | SKFIGI | Gene Therapy |
| | 830-844 | TEL | (2003) 10, 365-376. |
| | | | Blanco, Scientific |
| | | | Reports 7,40800 |
| | | | (2017); Sharma, J |
| | | | Immunol 1992; |
| | | | 148: 1499-1505 |
| 947TT | Tetanus | FNNFTV | CHEN et al, |
| | Toxoid | SFWLRV | Science China Life |
| | 947-967 | PKVSAS | Sciences 2014 57, |
| | | HLE | 117-127, |
| | | | Lanzavecchia, Eur. J. |
| | | | Immunol. 1989. |
| | | | 19: 2237-42; |
| | | | Amir-Kroll H, |
| | | | et al. J. |
| | | | Immunol. |
| | | | 170: 6165-6171 |
| | | | (2003) [symbol] |
| 1273TT | Tetanus | GQIGN | Corradin JI 1989, |
| | Toxoid | DPNR | 142: 394-402; |
| | 1273- | DIL | Muller 2012 PLoS |
| | 1284 | | ONE 7(5): e38329. |
| PADRE | Synthetic | AKFVA | Immunity. 1994 Dec; |
| | | AWTL | 1(9): 751-61. |
| | | KAAA | doi: 10.1016/s1074- |
| | | | 7613(94)80017. |

Glycopeptide designs comprising T-cell epitopes will be tested for antigenicity, immunogenicity and/or any other characteristics in any suitable in vitro and/or in vivo assays and studies. Such studies include without limitation various immunogenicity studies where these glycopeptide immunogens are administered as prime and/or boost. Non-limiting examples of such assays and studies are described in Example 1 and throughout the specification.

1. A recombinant fusion protein sequence comprising in order a V3 glycopeptide, a peptide linker and a self-assembling protein, wherein the fusion protein is comprised in a multimeric protein complex, wherein the fusion protein design is selected from the group consisting of: HV1301810, HV1301942, HV1301936, HV1301937, HV1301938, HV1301944, HV1301943, HV1301939, HV1301940, HV1301941, HV1302139, HV1302140, HV1302141, HV1302738 v2, HV1302739 v2, HV1302740 v2, HV1302741 v2, HV1302742 v2, HV1302743 v2, HV1302744 v2, HV1302745 v2, HV1302746 v2, HV1302747 v2, HV1302748 v2, HV1302749 v2, HV1302750 v2, HV1302751 V2, HV1302752 V2, HV1302753 V2, HV1302754 V2, HV1302755 V2, HV1302756 V2, HV1302757 V2, HV1302758 V2, HV1302759 V2, HV1302760 V2, and HV1302761 v2.

2. (canceled)

3. The recombinant fusion protein of claim 1 wherein the linker is LPXTGGGGGGSG or GSGLPXTGGGGG, wherein in some embodiments X is E.

4. The recombinant fusion protein of claim 1 wherein the linker is GGS (n), wherein n=1, 2, or 3.

5. The recombinant fusion protein of claim 1 wherein the linker is GGS(n) EKAAKAEEAAR(PP) the linker is EKAAKAEEAAR(PP) GGS (n) wherein n=1, 2, or 3.

6. The recombinant fusion protein of claim 1 wherein the protein further comprises a T helper epitope.

7. The recombinant fusion protein of claim 1 wherein the self-assembling protein is ferritin and the multimeric protein complex is ferritin nanoparticle.

8. A nucleic acid encoding the recombinant fusion protein sequence of claim 1.

9. The nucleic acid of claim 8 wherein the nucleic acid is an mRNA.

10. The recombinant fusion protein of claim 6, wherein, the T helper epitope is selected from the group consisting of: QYIKANSKFIGITEL, ENNFTVSFWLRVPKVSASHLE, GQIGNDPNRDIL, and AKFVAAWTLKAAA.

11.-14. (canceled)

15. A composition comprising the recombinant fusion protein of claim 1, or a portion thereof, and a pharmaceutically acceptable carrier.

16. A composition comprising the recombinant fusion protein of claim 7, or a portion thereof, and a pharmaceutically acceptable carrier.

17. A composition comprising a nucleic acid sequence encoding the recombinant fusion protein of claim 1, or a portion thereof, and a pharmaceutically acceptable carrier.

18. A virus-like particle comprising the recombinant fusion protein of of claim 1.

19. A host cell comprising a nucleic acid molecule encoding a recombinant fusion protein of claim 1.

20. An immunogenic composition comprising the recombinant fusion protein of claim 16, and a pharmaceutically acceptable carrier and an adjuvant.

21. A method for inducing an immune response to HIV-1 in a subject, comprising administering to the subject an effective amount of the recombinant fusion protein of claim 1, in an amount sufficient to induce an immune response.

22. The method of claim 21, wherein the effective amount of the recombinant fusion protein is administered as a boost.
